(19) United States
(12) Patent Application Publication
(10) Pub. No.: US 2024/0376228 A1
(43) Pub. Date: Nov. 14, 2024

(54) MULTI-SPECIFIC ANTIGEN-BINDING MOLECULE HAVING ALTERNATIVE FUNCTION TO FUNCTION OF BLOOD COAGULATION FACTOR VIII

(71) Applicant: Chugai Seiyaku Kabushiki Kaisha, Tokyo (JP)

(72) Inventors: Tomoyuki Igawa, Shizuoka (JP); Zenjiro Sampei, Shizuoka (JP); Tetsuo Kojima, Shizuoka (JP); Tetsuhiro Soeda, Shizuoka (JP); Atsushi Muto, Shizuoka (JP); Takehisa Kitazawa, Shizuoka (JP); Yukiko Nishida, Shizuoka (JP); Chifumi Imai, Shizuoka (JP); Tsukasa Suzuki, Shizuoka (JP); Kazutaka Yoshihashi, Shizuoka (JP)

(73) Assignee: Chugai Seiyaku Kabushiki Kaisha, Tokyo (JP)

(21) Appl. No.: 18/737,387

(22) Filed: Jun. 7, 2024

Related U.S. Application Data
(62) Division of application No. 18/495,861, filed on Oct. 27, 2023, now abandoned.
Application No. 18/495,861 is a division of application No. 18/081,874, filed on Dec. 15, 2022, now abandoned.
Application No. 18/081,874 is a division of application No. 17/729,471, filed on Apr. 26, 2022, now abandoned.
Application No. 17/729,471 is a division of application No. 17/485,818, filed on Sep. 27, 2021, now abandoned.
Application No. 17/485,818 is a division of application No. 16/459,791, filed on Jul. 2, 2019, now abandoned.
Application No. 16/459,791 is a continuation of application No. 15/288,965, filed on Oct. 7, 2016, now abandoned.
Application No. 15/288,965 is a division of application No. 15/132,996, filed on Apr. 19, 2016, now Pat. No. 10,450,381.
Application No. 15/132,996 is a division of application No. 13/885,421, filed on Aug. 30, 2013, now Pat. No. 9,334,331.

(30) Foreign Application Priority Data
Nov. 17, 2010 (JP) 2010-257022

(51) Int. Cl. C07K 16/40; C07K 16/36

(52) U.S. Cl. CPC C07K 16/40; C07K 16/36; C07K 2317/31; C07K 2317/51; C07K 2317/56; C07K 2317/565; C07K 2317/75; C07K 2317/76; C07K 2317/94

(57) ABSTRACT

Various bispecific antibodies that specifically bind to both blood coagulation factor IX/activated blood coagulation factor IX and blood coagulation factor X and functionally substitute for the cofactor function of blood coagulation factor VIII, that is, the function to promote activation of blood coagulation factor X by activated blood coagulation factor IX, were produced. From these antibodies, multispecific antigen-binding molecules having a high activity of functionally substituting for blood coagulation factor VIII were successfully discovered.

### CROSS-REFERENCE TO RELATED APPLICATIONS

This application is a divisional of U.S. application Ser. No. 18/495,861, filed on Oct. 27, 2023 (abandoned), which is a divisional of U.S. application Ser. No. 18/081,874, filed on Dec. 15, 2022 (abandoned), which is a divisional of U.S. application Ser. No. 17/729,471, filed on Apr. 26, 2022 (abandoned), which is a divisional of U.S. application Ser. No. 17/485,818, filed Sep. 27, 2021 (abandoned), which is a divisional of U.S. application Ser. No. 16/459,791, filed Jul. 2, 2019 (abandoned), which is a continuation of U.S. application Ser. No. 15/288,965, filed Oct. 7, 2016 (abandoned), which is a divisional of U.S. application Ser. No. 15/132,996, filed Apr. 19, 2016 (now U.S. Pat. No. 10,450,381, issued on Oct. 22, 2019), which is a divisional of U.S. application Ser. No. 13/885,421, filed Aug. 30, 2013 (now U.S. Pat. No. 9,334,331, issued on May 10, 2016), which is the National Stage of International Patent Application Serial No. PCT/JP2011/076486, filed Nov. 17, 2011, which claims the benefit of Japanese Patent Application Serial No. 2010-257022, filed on Nov. 17, 2010. The entire contents of U.S. application Ser. Nos. 18/495,861 and 18/081,874 are hereby incorporated by reference.

### SEQUENCE LISTING

This application contains a Sequence Listing that has been submitted electronically as an XML file named C1-A1007Psq.xml. The XML file, created on Jul. 30, 2024, is 242,961 bytes in size. The material in the XML file is hereby incorporated by reference in its entirety.

### TECHNICAL FIELD

The present invention relates to multispecific antigen-binding molecules that functionally substitute for blood coagulation factor VIII, a cofactor that enhances enzymatic reactions, and pharmaceutical compositions comprising such a molecule as an active ingredient.

### BACKGROUND ART

Hemophilia A is a bleeding abnormality caused by a hereditary decrease or deficiency of blood coagulation factor VIII (F.VIII) function. Hemophilia A patients are generally administered with an F.VIII formulation for the bleeding (on-demand administration). In recent years, F.VIII formulations are also administered prophylactically to prevent bleeding events (preventive administration: Non-patent Documents 1 and 2). The half-life of F.VIII formulations in blood is approximately 12 to 16 hours. Therefore, for continuous prevention, F. VIII formulations are administered to patients three times a week (Non-patent Documents 3 and 4). In on-demand administrations, F.VIII formulations are also additionally administered when necessary at regular intervals to prevent rebleeding. In addition, the administration of F.VIII formulations is done intravenously. Therefore, there has been a strong need for pharmaceutical agents with a lesser burden than F.VIII formulations.

Occasionally, anti-F.VIII antibodies (inhibitors) develop in hemophilia patients. Such inhibitors cancel the effects of the F.VIII formulations. For bleeding in patients who have developed inhibitors (inhibitor patients), bypass formulations are administered. Their action mechanisms are not dependent on F.VIII function, that is, the function of catalyzing the activation of blood coagulation factor X (F.X) by activated blood coagulation factor IX (F.IXa). Therefore, in some cases, bypass formulations cannot sufficiently stop the bleeding. Accordingly, there has been a strong need for pharmaceutical agents that are not affected by the presence of inhibitors and which can functionally substitute for F.VIII.

Recently, as a means for solving the problem, antibodies that functionally substitute for F.VIII and their use were disclosed (Patent Documents 1, 2, and 3). The antibodies may be effective for acquired hemophilia in which anti-F. VIII autoantibodies are present and for von Willebrand disease caused by an abnormality or deficiency of function of von Willebrand factor (vWF), but the activity of functionally substituting for F.VIII was not always sufficient. Therefore, as pharmaceutical agents exhibiting a high hemostatic effect, antibodies with a higher activity of functionally substituting for F.VIII than the above-mentioned antibodies were desired.

### PRIOR ART DOCUMENTS

### Patent Document

[Patent Document 1] WO 2005/035754
[Patent Document 2] WO 2005/035756
[Patent Document 3] WO 2006/109592

### Non-Patent Document

[Non-patent Document 1] Blood 58, 1-13 (1981)
[Non-patent Document 2] Nature 312, 330-337 (1984)
[Non-patent Document 3] Nature 312, 337-342 (1984)
[Non-patent Document 4] Biochim. Biophys. Acta 871, 268-278 (1986)

### SUMMARY OF THE INVENTION

### Problems to be Solved by the Invention

An objective of the present invention is to provide multispecific antigen-binding molecules that functionally substitute for F.VIII, a cofactor that enhances enzymatic reactions.

### Means for Solving the Problems

As a result of dedicated research, the present inventors succeeded in discovering bispecific antibodies having a better F.Xa generation-promoting activity than known antibodies from among various bispecific antibodies that specifically bind to both F.IX/F.IXa and F.X, and substitute for the cofactor function of F.VIII, that is, the function to promote F.X activation by F.IXa (F.Xa generation-promoting function).

Furthermore, the present inventors succeeded in finding the positions in the amino acid sequences of bispecific antibodies having the activity of functionally substituting for F.VIII that are important for improving the F.Xa generation-promoting activity of these antibodies, and thus they successfully obtained bispecific antibodies in which the activity of functionally substituting for F.VIII is further increased by replacing these amino acids. They also succeeded in obtaining bispecific antibodies which not only have a high activity of functionally substituting for F.VIII, but also have a low F.Xase inhibitory action. Satisfying both of these properties is very difficult.

Specifically, the present invention relates to multispecific antigen-binding molecules that functionally substitute for F.VIII, a cofactor that enhances enzymatic reactions, and pharmaceutical compositions comprising such a molecule as an active ingredient, and specifically relates to the following:
[1] a multispecific antigen-binding molecule that functionally substitutes for blood coagulation factor VIII, which comprises a first antigen-binding site that recognizes blood coagulation factor IX and/or activated blood coagulation factor IX and a second antigen-binding site that recognizes blood coagulation factor X, wherein the functional substitution for blood coagulation factor VIII results from an activated blood coagulation factor X (F.Xa) generation-promoting activity higher than the activity of a bispecific antibody (hA69-KQ/hB26-PF/hAL-AQ) which comprises an H chain comprising SEQ ID NOs: 165 and 166, and a commonly shared L chain comprising SEQ ID NO: 167;
[2] the multispecific antigen-binding molecule of [1], which comprises a first polypeptide comprising a first antigen-binding site that recognizes blood coagulation factor IX and/or activated blood coagulation factor IX and a third polypeptide comprising a third antigen-binding site that recognizes blood coagulation factor IX and/or activated blood coagulation factor IX, as well as a second polypeptide comprising a second antigen-binding site that recognizes blood coagulation factor X and a fourth polypeptide comprising a fourth antigen-binding site that recognizes blood coagulation factor X;
[3] the multispecific antigen-binding molecule of [2], wherein the first polypeptide and the third polypeptide each comprises an antigen-binding site of an H chain or L chain of an antibody against blood coagulation factor IX or activated blood coagulation factor IX, respectively; and the second polypeptide and the fourth polypeptide each comprises an antigen-binding site of an H chain or L chain of an antibody against blood coagulation factor X, respectively;
[4] the multispecific antigen-binding molecule of [3], wherein the antigen-binding site of the first polypeptide comprises an antigen-binding site which comprises H chain CDRs consisting of any one of the amino acid sequences selected from the following (a1) to (a11), or an antigen-binding site functionally equivalent thereto, and the antigen-binding site of the second polypeptide comprises an antigen-binding site which comprises H chain CDRs consisting of any one of the amino acid sequences selected from the following (b1) to (b11), or an antigen-binding site functionally equivalent thereto:
   (a1) an antigen-binding site comprising an H chain CDR 1, 2, and 3 amino acid sequences of SEQ ID NOs: 75, 76, and 77 (H chain CDRs of Q1), respectively;
   (a2) an antigen-binding site comprising an H chain CDR 1, 2, and 3 amino acid sequences of SEQ ID NOs: 78, 79, and 80 (H chain CDRs of Q31), respectively;
   (a3) an antigen-binding site comprising an H chain CDR 1, 2, and 3 amino acid sequences of SEQ ID NOs: 81, 82, and 83 (H chain CDRs of Q64), respectively;
   (a4) an antigen-binding site comprising an H chain CDR 1, 2, and 3 amino acid sequences of SEQ ID NOs: 84, 85, and 86 (H chain CDRs of Q85), respectively;
   (a5) an antigen-binding site comprising the H chain CDR 1, 2, and 3 amino acid sequences of SEQ ID NOs: 87, 88, and 89 (H chain CDRs of Q153), respectively;
   (a6) an antigen-binding site comprising an H chain CDR 1, 2, and 3 amino acid sequences of SEQ ID NOs: 90, 91, and 92 (H chain CDRs of Q354), respectively;
   (a7) an antigen-binding site comprising the H chain CDR 1, 2, and 3 amino acid sequences of SEQ ID NOs: 93, 94, and 95 (H chain CDRs of Q360), respectively;
   (a8) an antigen-binding site comprising the of H chain CDR 1, 2, and 3 amino acid sequences of SEQ ID NOs: 96, 97, and 98 (H chain CDRs of Q405), respectively;
   (a9) an antigen-binding site comprising an H chain CDR 1, 2, and 3 amino acid sequences of SEQ ID NOs: 99, 100, and 101 (H chain CDRs of Q458), respectively;
   (a10) an antigen-binding site comprising an H chain CDR 1, 2, and 3 amino acid sequences of SEQ ID NOs: 102, 103, and 104 (H chain CDRs of Q460), respectively;
   (a11) an antigen-binding site comprising an H chain CDR 1, 2, and 3 amino acid sequences of SEQ ID NOs: 105, 106, and 107 (H chain CDRs of Q499), respectively;
   (b1) an antigen-binding site comprising an H chain CDR 1, 2, and 3 amino acid sequences of SEQ ID NOs: 108, 109, and 110 (H chain CDRs of J232), respectively;
   (b2) an antigen-binding site comprising an H chain CDR 1, 2, and 3 amino acid sequences of SEQ ID NOs: 111, 112, and 113 (H chain CDRs of J259), respectively;
   (b3) an antigen-binding site comprising an H chain CDR 1, 2, and 3 amino acid sequences of SEQ ID NOs: 114, 115, and 116 (H chain CDRs of J268), respectively;
   (b4) an antigen-binding site comprising an H chain CDR 1, 2, and 3 amino acid sequences of SEQ ID NOs: 117, 118, and 119 (H chain CDRs of J300), respectively;
   (b5) an antigen-binding site comprising an H chain CDR 1, 2, and 3 amino acid sequences of SEQ ID NOs: 120, 121, and 122 (H chain CDRs of J321), respectively;
   (b6) an antigen-binding site comprising the H chain CDR 1, 2, and 3 amino acid sequences of SEQ ID NOs: 123, 124, and 125 (H chain CDRs of J326), respectively;
   (b7) an antigen-binding site comprising an H chain CDR 1, 2, and 3 amino acid sequences of SEQ ID NOs: 126, 127, and 128 (H chain CDRs of J327), respectively;
   (b8) an antigen-binding site comprising an H chain CDR 1, 2, and 3 amino acid sequences of SEQ ID NOs: 129, 130, and 131 (H chain CDRs of J339), respectively;
   (b9) an antigen-binding site comprising an H chain CDR 1, 2, and 3 amino acid sequences of SEQ ID NOs: 132, 133, and 134 (H chain CDRs of J344), respectively;
   (b10) an antigen-binding site comprising an H chain CDR 1, 2, and 3 amino acid sequences of SEQ ID NOs: 135, 136, and 137 (H chain CDRs of J346), respectively; and
   (b11) an antigen-binding site comprising an H chain CDR 1, 2, and 3 amino acid sequences of SEQ ID NOs: 174, 175, and 176 (H chain CDRs of J142), respectively;
[5] the multispecific antigen-binding molecule of [3], wherein the antigen-binding site of the first polypeptide comprises an antigen-binding site which comprises an H chain variable region consisting of any one of the amino acid sequences selected from the following (a1) to (a11), or an antigen-binding site functionally equivalent thereto, and the antigen-binding site of the second polypeptide comprises an antigen-binding site which comprises an H chain variable region consisting of any one of the amino acid sequences selected from the following (b1) to (b11), or an antigen-binding site functionally equivalent thereto:
   (a1) an antigen-binding site comprising an H chain variable region amino acid sequence of SEQ ID NO: 35 (H chain variable region of Q64);
   (a2) an antigen-binding site comprising an H chain variable region amino acid sequence of SEQ ID NO: 36 (H chain variable region of Q31);
   (a3) an antigen-binding site comprising an H chain variable region amino acid sequence of SEQ ID NO: 37 (H chain variable region of Q1);
   (a4) an antigen-binding site comprising an H chain variable region amino acid sequence of SEQ ID NO: 38 (H chain variable region of Q85);
   (a5) an antigen-binding site comprising an H chain variable region amino acid sequence of SEQ ID NO: 39 (H chain variable region of Q153);
   (a6) an antigen-binding site comprising an H chain variable region amino acid sequence of SEQ ID NO: 40 (H chain variable region of Q354);
   (a7) an antigen-binding site comprising an H chain variable region amino acid sequence of SEQ ID NO: 41 (H chain variable region of Q360);
   (a8) an antigen-binding site comprising an H chain variable region amino acid sequence of SEQ ID NO: 42 (H chain variable region of Q405);
   (a9) an antigen-binding site comprising an H chain variable region amino acid sequence of SEQ ID NO: 43 (H chain variable region of Q458);
   (a10) an antigen-binding site comprising an H chain variable region amino acid sequence of SEQ ID NO: 44 (H chain variable region of Q460);
   (a11) an antigen-binding site comprising an H chain variable region amino acid sequence of SEQ ID NO: 45 (H chain variable region of Q499);
   (b1) an antigen-binding site comprising an H chain variable region amino acid sequence of SEQ ID NO: 46 (H chain variable region of J232);
   (b2) an antigen-binding site comprising an H chain variable region amino acid sequence of SEQ ID NO: 47 (H chain variable region of J259);
   (b3) an antigen-binding site comprising an H chain variable region amino acid sequence of SEQ ID NO: 48 (H chain variable region of J268);
   (b4) an antigen-binding site comprising an H chain variable region amino acid sequence of SEQ ID NO: 49 (H chain variable region of J300);
   (b5) an antigen-binding site comprising an H chain variable region amino acid sequence of SEQ ID NO: 50 (H chain variable region of J321);
   (b6) an antigen-binding site comprising an H chain variable region amino acid sequence of SEQ ID NO: 51 (H chain variable region of J326);
   (b7) an antigen-binding site comprising an H chain variable region amino acid sequence of SEQ ID NO: 52 (H chain variable region of J327);
   (b8) an antigen-binding site comprising an H chain variable region amino acid sequence of SEQ ID NO: 53 (H chain variable region of J339);
   (b9) an antigen-binding site comprising an H chain variable region amino acid sequence of SEQ ID NO: 54 (H chain variable region of J344);
   (b10) an antigen-binding site comprising an H chain variable region amino acid sequence of SEQ ID NO: 55 (H chain variable region of J346); and
   (b11) an antigen-binding site comprising an H chain variable region amino acid sequence of SEQ ID NO: 172 (H chain variable region of J142);
[6] the multispecific antigen-binding molecule of [3], wherein the antigen-binding sites included in the third polypeptide and the fourth polypeptide comprise an antigen-binding site which comprises L chain CDRs consisting of any one of the amino acid sequences selected from the following (c1) to (c10), or an antigen-binding site functionally equivalent thereto:
   (c1) an antigen-binding site comprising an L chain CDR1, 2, and 3 amino acid sequences of SEQ ID NOs: 138, 139, and 140 (L chain CDR of L2), respectively;
   (c2) an antigen-binding site comprising an L chain CDR1, 2, and 3 amino acid sequences of SEQ ID NOs: 141, 142, and 143 (L chain CDR of L45), respectively;
   (c3) an antigen-binding site comprising an L chain CDR1, 2, and 3 amino acid sequences of SEQ ID NOs: 144, 145, and 146 (L chain CDR of L248), respectively;
   (c4) an antigen-binding site comprising an L chain CDR1, 2, and 3 amino acid sequences of SEQ ID NOs: 147, 148, and 149 (L chain CDR of L324), respectively;
   (c5) an antigen-binding site comprising an L chain CDR1, 2, and 3 amino acid sequences of SEQ ID NOs: 150, 151, and 152 (L chain CDR of L334), respectively;
   (c6) an antigen-binding site comprising an L chain CDR1, 2, and 3 amino acid sequences of SEQ ID NOs: 153, 154, and 155 (L chain CDR of L377), respectively;
   (c7) an antigen-binding site comprising an L chain CDR1, 2, and 3 amino acid sequences of SEQ ID NOs: 156, 157, and 158 (L chain CDR of L404), respectively;
   (c8) an antigen-binding site comprising an L chain CDR1, 2, and 3 amino acid sequences of SEQ ID NOs: 159, 160, and 161 (L chain CDR of L406), respectively;
   (c9) an antigen-binding site comprising an L chain CDR1, 2, and 3 amino acid sequences of SEQ ID NOs: 137, 138, and 139 (L chain CDR of L408), respectively; and
   (c10) an antigen-binding site comprising an L chain CDR1, 2, and 3 amino acid sequences of SEQ ID NOs: 177, 178, and 179 (L chain CDR of L180), respectively;
[7] the multispecific antigen-binding molecule of [3], wherein the antigen-binding sites included in the third polypeptide and the fourth polypeptide comprise an antigen-binding site which comprises an L chain variable region consisting of any one of the amino acid sequences selected from the following (c1) to (c10), or an antigen-binding site functionally equivalent thereto:
   (c1) an antigen-binding site comprising an L chain variable region amino acid sequence of SEQ ID NO: 56 (L chain variable region of L2);
   (c2) an antigen-binding site comprising an L chain variable region amino acid sequence of SEQ ID NO: 57 (L chain variable region of L45);
   (c3) an antigen-binding site comprising an L chain variable region amino acid sequence of SEQ ID NO: 58 (L chain variable region of L248);
   (c4) an antigen-binding site comprising an L chain variable region amino acid sequence of SEQ ID NO: 59 (L chain variable region of L324);
   (c5) an antigen-binding site comprising an L chain variable region amino acid sequence of SEQ ID NO: 60 (L chain variable region of L334);
   (c6) an antigen-binding site comprising an L chain variable region amino acid sequence of SEQ ID NO: 61 (L chain variable region of L377);
   (c7) an antigen-binding site comprising an L chain variable region amino acid sequence of SEQ ID NO: 62 (L chain variable region of L404);
   (c8) an antigen-binding site comprising an L chain variable region amino acid sequence of SEQ ID NO: 63 (L chain variable region of L406);
   (c9) an antigen-binding site comprising an L chain variable region amino acid sequence of SEQ ID NO: 64 (L chain variable region of L408); and
   (c10) an antigen-binding site comprising an L chain variable region amino acid sequence of SEQ ID NO: 173 (L chain variable region of L180);
[8] the multispecific antigen-binding molecule of [3], wherein the first and second polypeptides further comprise an antibody H chain constant region, and the third and fourth polypeptides comprise an antibody L chain constant region;
[9] the multispecific antigen-binding molecule of [3], wherein the first and second polypeptides comprise an antibody H chain constant region, and the third and fourth polypeptides comprise an antibody L chain constant region, and wherein the third polypeptide and the fourth polypeptide are a commonly shared L chain;
[10] the multispecific antigen-binding molecule of [8] or [9], wherein the first polypeptide comprises an antibody H chain constant region consisting of any one of the amino acid sequences selected from the group consisting of the following (d1) to (d6) or the group consisting of the following (d7) to (d9), and the second polypeptide comprises an antibody H chain constant region consisting of any one of the amino acid sequences selected from a group different from that of the above-mentioned first polypeptide:
   (d1) an H chain constant region of SEQ ID NO: 65 (G4k);
   (d2) an H chain constant region of SEQ ID NO: 66 (z7);
   (d3) an H chain constant region of SEQ ID NO: 67 (z55);
   (d4) an H chain constant region of SEQ ID NO: 68 (z106);
   (d5) an H chain constant region of SEQ ID NO: 69 (z118);
   (d6) an H chain constant region of SEQ ID NO: 70 (z121);
   (d7) an H chain constant region of SEQ ID NO: 71 (G4h);
   (d8) an H chain constant region of SEQ ID NO: 72 (z107); and
   (d9) an H chain constant region of SEQ ID NO: 73 (z119);
[11] the multispecific antigen-binding molecule of [8] or [9], wherein the third and fourth polypeptides comprise the antibody L chain constant region consisting of the following amino acid sequence of:
   (e) an L chain constant region of SEQ ID NO: 74 (k);
[12] the multispecific antigen-binding molecule of [8] or [9], wherein the first polypeptide comprises any one antibody H chain selected from the following (a1) to (a14), the second polypeptide comprises any one antibody H chain selected from the following (b1) to (b12), and the third polypeptide and the fourth polypeptide comprise any one antibody L chain selected from the following (c1) to (c10):
   (a1) an antibody H chain consisting of the amino acid sequence of SEQ ID NO: 1 (Q1-G4k);
   (a2) an antibody H chain consisting of the amino acid sequence of SEQ ID NO: 2 (Q31-z7);
   (a3) an antibody H chain consisting of the amino acid sequence of SEQ ID NO: 3 (Q64-z55);
   (a4) an antibody H chain consisting of the amino acid sequence of SEQ ID NO: 10 (Q64-z7);
   (a5) an antibody H chain consisting of the amino acid sequence of SEQ ID NO: 11 (Q85-G4k);
   (a6) an antibody H chain consisting of the amino acid sequence of SEQ ID NO: 12 (Q153-G4k);
   (a7) an antibody H chain consisting of the amino acid sequence of SEQ ID NO: 13 (Q354-z106);
   (a8) an antibody H chain consisting of the amino acid sequence of SEQ ID NO: 14 (Q360-G4k);
   (a9) an antibody H chain consisting of the amino acid sequence of SEQ ID NO: 15 (Q360-z118);
   (a10) an antibody H chain consisting of the amino acid sequence of SEQ ID NO: 16 (Q405-G4k);
   (a11) an antibody H chain consisting of the amino acid sequence of SEQ ID NO: 17 (Q458-z106);
   (a12) an antibody H chain consisting of the amino acid sequence of SEQ ID NO: 18 (Q460-z121);
   (a13) an antibody H chain consisting of the amino acid sequence of SEQ ID NO: 19 (Q499-z118);
   (a14) an antibody H chain consisting of the amino acid sequence of SEQ ID NO: 20 (Q499-z121);
   (b1) an antibody H chain consisting of the amino acid sequence of SEQ ID NO: 4 (J268-G4h);
   (b2) an antibody H chain consisting of the amino acid sequence of SEQ ID NO: 5 (J321-G4h);
   (b3) an antibody H chain consisting of the amino acid sequence of SEQ ID NO: 6 (J326-z107);
   (b4) an antibody H chain consisting of the amino acid sequence of SEQ ID NO: 7 (J344-z107);
   (b5) an antibody H chain consisting of the amino acid sequence of SEQ ID NO: 21 (J232-G4h);
   (b6) an antibody H chain consisting of the amino acid sequence of SEQ ID NO: 22 (J259-z107);
   (b7) an antibody H chain consisting of the amino acid sequence of SEQ ID NO: 23 (J300-z107);
   (b8) an antibody H chain consisting of the amino acid sequence of SEQ ID NO: 24 (J327-z107);
   (b9) an antibody H chain consisting of the amino acid sequence of SEQ ID NO: 25 (J327-z119);
   (b10) an antibody H chain consisting of the amino acid sequence of SEQ ID NO: 26 (J339-z119);
   (b11) an antibody H chain consisting of the amino acid sequence of SEQ ID NO: 27 (J346-z107);
   (b12) an antibody H chain consisting of the amino acid sequence of SEQ ID NO: 170 (J142-G4h);
   (c1) an antibody L chain consisting of the amino acid sequence of SEQ ID NO: 8 (L2-k);
   (c2) an antibody L chain consisting of the amino acid sequence of SEQ ID NO: 9 (L45-k);
   (c3) an antibody L chain consisting of the amino acid sequence of SEQ ID NO: 28 (L248-k);
   (c4) an antibody L chain consisting of the amino acid sequence of SEQ ID NO: 29 (L324-k);
   (c5) an antibody L chain consisting of the amino acid sequence of SEQ ID NO: 30 (L334-k);
   (c6) an antibody L chain consisting of the amino acid sequence of SEQ ID NO: 31 (L377-k);
   (c7) an antibody L chain consisting of the amino acid sequence of SEQ ID NO: 32 (L404-k);
   (c8) an antibody L chain consisting of the amino acid sequence of SEQ ID NO: 33 (L406-k);
   (c9) an antibody L chain consisting of the amino acid sequence of SEQ TD NO: 34 (L408-k); and
   (c10) an antibody L chain consisting of the amino acid sequence of SEQ ID NO: 171 (L180-k);
[13] the multispecific antigen-binding molecule of [1], wherein the first polypeptide comprises an antigen-binding site which binds to an epitope overlapping with an epitope that binds to an antibody consisting of the antibody H chain of any one of (a1) to (a14) and the antibody L chain of any one of (c1) to (c10) of [12], and the second polypeptide comprises an antigen-binding site which binds to an epitope overlapping with an epitope that binds to an antibody consisting of the antibody H chain of any one of (b1) to (b12) and the antibody L chain of any one of (c1) to (c10) of [12];
[14] the multispecific antigen-binding molecule of [8] or [9], wherein the first polypeptide comprises any one antibody H chain selected from the following (e1) to (e3), the second polypeptide comprises any one antibody H chain selected from the following (f1) to (f3), and the third polypeptide and the fourth polypeptide comprise any one antibody L chain selected from the following (g1) to (g4):
   (e1) an H chain of an antibody which binds to an epitope overlapping with an epitope bound by an antibody consisting of an antibody H chain of any one of (a1) to (a14) and an antibody L chain of any one of (c1) to (c10), of [12];
   (e2) an antibody H chain, wherein at least one amino acid residue selected from the amino acid residues at positions 34, 35, 49, 61, 62, 96, 98, 100, 100b, and 102 by Kabat numbering in any one antibody H chain selected from (e1) is substituted with another amino acid;
   (e3) an antibody H chain, wherein by Kabat numbering, the amino acid residue at position 34 is isoleucine, the amino acid residue at position 35 is asparagine, glutamine, or serine, the amino acid residue at position 49 is serine, the amino acid residue at position 61 is arginine, the amino acid residue at position 62 is glutamic acid, the amino acid residue at position 96 is serine or threonine, the amino acid residue at position 98 is lysine or arginine, the amino acid residue at position 100 is phenylalanine or tyrosine, the amino acid residue at position 100b is glycine, or the amino acid residue at position 102 is tyrosine in any antibody H chain selected from (e1);
   (f1) an H chain of an antibody which binds to an epitope overlapping with an epitope bound by an antibody consisting of an antibody H chain of any of (b1) to (b12) of [12] and an antibody L chain of any of (c1) to (c10) of [12];
   (f2) an antibody H chain, wherein at least one amino acid residue selected from the amino acid residues at positions 35, 53, 73, 76, 96, 98, 100, and 100a by Kabat numbering in any antibody H chain of (f1) is substituted with another amino acid;
   (f3) an antibody H chain, wherein by Kabat numbering, the amino acid residue at position 35 is aspartic acid, the amino acid residue at position 53 is arginine, the amino acid residue at position 73 is lysine, the amino acid residue at position 76 is glycine, the amino acid residue at position 96 is lysine or arginine, the amino acid residue at position 98 is tyrosine, the amino acid residue at position 100 is tyrosine, or the amino acid residue at position 100a is histidine in any one antibody H chain selected from
   (f1); (g1) an L chain of an antibody which binds to an epitope overlapping with an epitope bound by an antibody which consists of an antibody H chain of any one of (a1) to
   (a14) and an antibody L chain of any one of (c1) to (c10), of [12]; (g2) an L chain of an antibody which binds to an epitope overlapping with an epitope bound by an antibody which consists of an antibody H chain of any one of (b1) to (b12) and an antibody L chain of any one of (c1) to (c10), of [12];
   (g3) an antibody L chain, wherein at least one amino acid residue selected from the amino acid residues at positions 27, 30, 31, 32, 50, 52, 53, 54, 55, 92, 93, 94, and 95 by Kabat numbering in the antibody L chain of either (g1) or (g2) is substituted with another amino acid; and
   (g4) an antibody L chain, wherein by Kabat numbering, the amino acid residue at position 27 is lysine or arginine, the amino acid residue at position 30 is glutamic acid, the amino acid residue at position 31 is arginine, the amino acid residue at position 32 is glutamine, the amino acid residue at position 50 is arginine or glutamine, the amino acid residue at position 52 is serine, the amino acid residue at position 53 is arginine, the amino acid residue at position 54 is lysine, the amino acid residue at position 55 is glutamic acid, the amino acid residue at position 92 is serine, the amino acid residue at position 93 is serine, the amino acid residue at position 94 is proline, or the amino acid residue at position 95 is proline in the antibody L chain of either (g1) or (g2);
[15] the multispecific antigen-binding molecule of any one of [1] to [14], wherein the multispecific antigen-binding molecule is a multispecific antibody;
[16] a bispecific antibody of any one of the following (a) to (u):
   (a) a bispecific antibody (Q1-G4k/J268-G4h/L45-k), wherein the first polypeptide is an H chain consisting of the amino acid sequence of SEQ ID NO: 1, the second polypeptide is an H chain consisting of the amino acid sequence of SEQ ID NO: 4, and the third polypeptide and the fourth polypeptide are a commonly shared L chain of SEQ ID NO: 9;
   (b) a bispecific antibody (Q1-G4k/J321-G4h/L45-k), wherein the first polypeptide is an H chain consisting of the amino acid sequence of SEQ ID NO: 1, the second polypeptide is an H chain consisting of the amino acid sequence of SEQ ID NO: 5, and the third polypeptide and the fourth polypeptide are a commonly shared L chain of SEQ ID NO: 9;
   (c) a bispecific antibody (Q31-z7/J326-z107/L2-k), wherein the first polypeptide is an H chain consisting of the amino acid sequence of SEQ ID NO: 2, the second polypeptide is an H chain consisting of the amino acid sequence of SEQ ID NO: 6, and the third polypeptide and the fourth polypeptide are a commonly shared L chain of SEQ ID NO: 8;
   (d) a bispecific antibody (Q64-z55/J344-z107/L45-k), wherein the first polypeptide is an H chain consisting of the amino acid sequence of SEQ ID NO: 3, the second polypeptide is an H chain consisting of the amino acid sequence of SEQ ID NO: 7, and the third polypeptide and the fourth polypeptide are a commonly shared L chain of SEQ ID NO: 9;
   (e) a bispecific antibody (Q64-z7/J326-z107/L334-k), wherein the first polypeptide is an H chain consisting of the amino acid sequence of SEQ ID NO: 10, the second polypeptide is an H chain consisting of the amino acid sequence of SEQ ID NO: 6, and the third polypeptide and the fourth polypeptide are a commonly shared L chain of SEQ ID NO: 30;
   (f) a bispecific antibody (Q64-z7/J344-z107/L406-k), wherein the first polypeptide is an H chain consisting of the amino acid sequence of SEQ ID NO: 10, the second polypeptide is an H chain consisting of the amino acid sequence of SEQ ID NO: 7, and the third polypeptide and the fourth polypeptide are a commonly shared L chain of SEQ ID NO: 33;
   (g) a bispecific antibody (Q85-G4k/J268-G4h/L406-k), wherein the first polypeptide is an H chain consisting of the amino acid sequence of SEQ ID NO: 11, the second polypeptide is an H chain consisting of the amino acid sequence of SEQ ID NO: 4, and the third polypeptide and the fourth polypeptide are a commonly shared L chain of SEQ ID NO: 33;
   (h) a bispecific antibody (Q85-G4k/J321-G4h/L334-k), wherein the first polypeptide is an H chain consisting of the amino acid sequence of SEQ ID NO: 11, the second polypeptide is an H chain consisting of the amino acid sequence of SEQ ID NO: 5, and the third polypeptide and the fourth polypeptide are a commonly shared L chain of SEQ ID NO: 30;
   (i) a bispecific antibody (Q153-G4k/J232-G4h/L406-k), wherein the first polypeptide is an H chain consisting of the amino acid sequence of SEQ ID NO: 12, the second polypeptide is an H chain consisting of the amino acid sequence of SEQ ID NO: 21, and the third polypeptide and the fourth polypeptide are a commonly shared L chain of SEQ ID NO: 33;
   (j) a bispecific antibody (Q354-z106/J259-z107/L324-k), wherein the first polypeptide is an H chain consisting of the amino acid sequence of SEQ ID NO: 13, the second polypeptide is an H chain consisting of the amino acid sequence of SEQ ID NO: 22, and the third polypeptide and the fourth polypeptide are a commonly shared L chain of SEQ ID NO: 29;
   (k) a bispecific antibody (Q360-G4k/J232-G4h/L406-k), wherein the first polypeptide is an H chain consisting of the amino acid sequence of SEQ ID NO: 14, the second polypeptide is an H chain consisting of the amino acid sequence of SEQ ID NO: 21, and the third polypeptide and the fourth polypeptide are a commonly shared L chain of SEQ ID NO: 33;
   (1) a bispecific antibody (Q360-z118/J300-z107/L334-k), wherein the first polypeptide is an H chain consisting of the amino acid sequence of SEQ ID NO: 15, the second polypeptide is an H chain consisting of the amino acid sequence of SEQ ID NO: 23, and the third polypeptide and the fourth polypeptide are a commonly shared L chain of SEQ ID NO: 30;
   (m) a bispecific antibody (Q405-G4k/J232-G4h/L248-k), wherein the first polypeptide is an H chain consisting of the amino acid sequence of SEQ ID NO: 16, the second polypeptide is an H chain consisting of the amino acid sequence of SEQ ID NO: 21, and the third polypeptide and the fourth polypeptide are a commonly shared L chain of SEQ ID NO: 28;
   (n) a bispecific antibody (Q458-z106/J346-z107/L408-k), wherein the first polypeptide is an H chain consisting of the amino acid sequence of SEQ ID NO: 17, the second polypeptide is an H chain consisting of the amino acid sequence of SEQ ID NO: 27, and the third polypeptide and the fourth polypeptide are a commonly shared L chain of SEQ ID NO: 34;
   (o) a bispecific antibody (Q460-z121/J327-z119/L334-k), wherein the first polypeptide is an H chain consisting of the amino acid sequence of SEQ ID NO: 18, the second polypeptide is an H chain consisting of the amino acid sequence of SEQ ID NO: 25, and the third polypeptide and the fourth polypeptide are a commonly shared L chain of SEQ ID NO: 30;
   (p) a bispecific antibody (Q499-z118/J327-z107/L334-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 30;
   (q) a bispecific antibody (Q499-z118/J327-z107/L377-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 31;
   (r) a bispecific antibody (Q499-z118/J346-z107/L248-k), wherein the first polypeptide is an H chain consisting of the amino acid sequence of SEQ ID NO: 19, the second polypeptide is an H chain consisting of the amino acid sequence of SEQ ID NO: 27, and the third polypeptide and the fourth polypeptide are a commonly shared L chain of SEQ ID NO: 28;
   (s) a bispecific antibody (Q499-z121/J327-z119/L404-k), wherein the first polypeptide is an H chain consisting of the amino acid sequence of SEQ ID NO: 20, the second polypeptide is an H chain consisting of the amino acid sequence of SEQ ID NO: 25, and the third polypeptide and the fourth polypeptide are a commonly shared L chain of SEQ ID NO: 32;
   (t) a bispecific antibody (Q499-z121/J339-z119/L377-k), wherein the first polypeptide is an H chain consisting of the amino acid sequence of SEQ ID NO: 20, the second polypeptide is an H chain consisting of the amino acid sequence of SEQ ID NO: 26, and the third polypeptide and the fourth polypeptide are a commonly shared L chain of SEQ ID NO: 31; and
   (u) a bispecific antibody (Q153-G4k/J142-G4h/L180-k), wherein the first polypeptide is an H chain consisting of the amino acid sequence of SEQ ID NO: 12, the second polypeptide is an H chain consisting of the amino acid sequence of SEQ ID NO: 170, and the third polypeptide and the fourth polypeptide are a commonly shared L chain of SEQ ID NO: 171;
[17] a nucleic acid encoding the multispecific antigen-binding molecule of any one of [1] to [15] or the bispecific antibody of [16];
[18] a vector inserted with the nucleic acid of [17];
[19] a cell comprising the nucleic acid of [17] or the vector of [18];
[20] a method for producing the multispecific antigen-binding molecule of any one of [1] to [15] or the bispecific antibody of [16] by culturing the cell of [19];
[21] a pharmaceutical composition comprising the multispecific antigen-binding molecule of any one of [1] to [15] or the bispecific antibody of [16], and a pharmaceutically acceptable carrier;
[22] the composition of [21], which is a pharmaceutical composition used for prevention and/or treatment of bleeding, a disease accompanying bleeding, or a disease caused by bleeding;
[23] the composition of [22], wherein the bleeding, the disease accompanying bleeding, or the disease caused by bleeding is a disease that develops and/or progresses due to a decrease or deficiency in the activity of blood coagulation factor VIII and/or activated blood coagulation factor VIII;
[24] the composition of [23], wherein the disease that develops and/or progresses due to a decrease or deficiency in the activity of blood coagulation factor VIII and/or activated blood coagulation factor VIII is hemophilia A;
[25] the composition of [23], wherein the disease that develops and/or progresses due to a decrease or deficiency in the activity of blood coagulation factor VIII and/or activated blood coagulation factor VIII is a disease showing emergence of an inhibitor against blood coagulation factor VIII and/or activated blood coagulation factor VIII;
[26] the composition of [23], wherein the disease that develops and/or progresses due to a decrease or deficiency in the activity of blood coagulation factor VIII and/or activated blood coagulation factor VIII is acquired hemophilia;
[27] the composition of [23], wherein the disease that develops and/or progresses due to a decrease in the activity of blood coagulation factor VIII and/or activated blood coagulation factor VIII is von Willebrand disease;
[28] a method for preventing and/or treating bleeding, a disease accompanying bleeding, or a disease caused by bleeding, which comprises the step of administering the multispecific antigen-binding molecule of any one of [1] to [15] or the bispecific antibody of [16], or the composition of any one of [21] to [27]; and
[29] a kit for use in the prevention and/or treatment method of [28], which comprises at least the multispecific antigen-binding molecule of any one of [1] to [15] or the bispecific antibody of [16], or the composition of any one of [21] to [27].

Furthermore, the present invention relates to:
[30] use of the multispecific antigen-binding molecule of any one of [1] to [15], the bispecific antibody of [16], or the composition of any one of [21] to [27] in the manufacture of an agent for preventing and/or treating bleeding, a disease accompanying bleeding, or a disease caused by bleeding; and
[31] the multispecific antigen-binding molecule of any one of [1] to [15], the bispecific antibody of [16], or the composition of any one of [21] to [27] for preventing and/or treating bleeding, a disease accompanying bleeding, or a disease caused by bleeding.

The present invention also relates to bispecific antibodies that functionally substitute for F.VIII, a cofactor that enhances enzymatic reactions, and pharmaceutical compositions comprising the antibody as an active ingredient, and more specifically relates to:
[32] a bispecific antibody that functionally substitutes for blood coagulation factor VIII, which comprises a first antigen-binding site that recognizes blood coagulation factor IX and/or activated blood coagulation factor IX and a second antigen-binding site that recognizes blood coagulation factor X, wherein the bispecific antibody is any of the following (a) to (u):
   (a) a bispecific antibody (Q1-G4k/J268-G4h/L45-k), wherein the first polypeptide is an H chain consisting of the amino acid sequence of SEQ ID NO: 1, the second polypeptide is an H chain consisting of the amino acid sequence of SEQ ID NO: 4, and the third polypeptide and the fourth polypeptide are a commonly shared L chain of SEQ ID NO: 9;
   (b) a bispecific antibody (Q1-G4k/J321-G4h/L45-k), wherein the first polypeptide is an H chain consisting of the amino acid sequence of SEQ ID NO: 1, the second polypeptide is an H chain consisting of the amino acid sequence of SEQ ID NO: 5, and the third polypeptide and the fourth polypeptide are a commonly shared L chain of SEQ ID NO: 9;
   (c) a bispecific antibody (Q31-z7/J326-z107/L2-k), wherein the first polypeptide is an H chain consisting of the amino acid sequence of SEQ ID NO: 2, the second polypeptide is an H chain consisting of the amino acid sequence of SEQ ID NO: 6, and the third polypeptide and the fourth polypeptide are a commonly shared L chain of SEQ ID NO: 8;
   (d) a bispecific antibody (Q64-z55/J344-z107/L45-k), wherein the first polypeptide is an H chain consisting of the amino acid sequence of SEQ ID NO: 3, the second polypeptide is an H chain consisting of the amino acid sequence of SEQ ID NO: 7, and the third polypeptide and the fourth polypeptide are a commonly shared L chain of SEQ ID NO: 9;
   (e) a bispecific antibody (Q64-z7/J326-z107/L334-k), wherein the first polypeptide is an H chain consisting of the amino acid sequence of SEQ ID NO: 10, the second polypeptide is an H chain consisting of the amino acid sequence of SEQ ID NO: 6, and the third polypeptide and the fourth polypeptide are a commonly shared L chain of SEQ ID NO: 30;
   (f) a bispecific antibody (Q64-z7/J344-z107/L406-k), wherein the first polypeptide is an H chain consisting of the amino acid sequence of SEQ ID NO: 10, the second polypeptide is an H chain consisting of the amino acid sequence of SEQ ID NO: 7, and the third polypeptide and the fourth polypeptide are a commonly shared L chain of SEQ ID NO: 33;
   (g) a bispecific antibody (Q85-G4k/J268-G4h/L406-k), wherein the first polypeptide is an H chain consisting of the amino acid sequence of SEQ ID NO: 11, the second polypeptide is an H chain consisting of the amino acid sequence of SEQ ID NO: 4, and the third polypeptide and the fourth polypeptide are a commonly shared L chain of SEQ ID NO: 33;
   (h) a bispecific antibody (Q85-G4k/J321-G4h/L334-k), wherein the first polypeptide is an H chain consisting of the amino acid sequence of SEQ ID NO: 11, the second polypeptide is an H chain consisting of the amino acid sequence of SEQ ID NO: 5, and the third polypeptide and the fourth polypeptide are a commonly shared L chain of SEQ ID NO: 30;
   (i) a bispecific antibody (Q153-G4k/J232-G4h/L406-k), wherein the first polypeptide is an H chain consisting of the amino acid sequence of SEQ ID NO: 12, the second polypeptide is an H chain consisting of the amino acid sequence of SEQ ID NO: 21, and the third polypeptide and the fourth polypeptide are a commonly shared L chain of SEQ ID NO: 33;
   (j) a bispecific antibody (Q354-z106/J259-z107/L324-k), wherein the first polypeptide is an H chain consisting of the amino acid sequence of SEQ ID NO: 13, the second polypeptide is an H chain consisting of the amino acid sequence of SEQ ID NO: 22, and the third polypeptide and the fourth polypeptide are a commonly shared L chain of SEQ ID NO: 29;
   (k) a bispecific antibody (Q360-G4k/J232-G4h/L406-k), wherein the first polypeptide is an H chain consisting of the amino acid sequence of SEQ ID NO: 14, the second polypeptide is an H chain consisting of the amino acid sequence of SEQ ID NO: 21, and the third polypeptide and the fourth polypeptide are a commonly shared L chain of SEQ ID NO: 33;
   (l) a bispecific antibody (Q360-z118/J300-z107/L334-k), wherein the first polypeptide is an H chain consisting of the amino acid sequence of SEQ ID NO: 15, the second polypeptide is an H chain consisting of the amino acid sequence of SEQ ID NO: 23, and the third polypeptide and the fourth polypeptide are a commonly shared L chain of SEQ ID NO: 30;
   (m) a bispecific antibody (Q405-G4k/J232-G4h/L248-k), wherein the first polypeptide is an H chain consisting of the amino acid sequence of SEQ ID NO: 16, the second polypeptide is an H chain consisting of the amino acid sequence of SEQ ID NO: 21, and the third polypeptide and the fourth polypeptide are a commonly shared L chain of SEQ ID NO: 28;
   (n) a bispecific antibody (Q458-z106/J346-z107/L408-k), wherein the first polypeptide is an H chain consisting of the amino acid sequence of SEQ ID NO: 17, the second polypeptide is an H chain consisting of the amino acid sequence of SEQ ID NO: 27, and the third polypeptide and the fourth polypeptide are a commonly shared L chain of SEQ ID NO: 34;
   (o) a bispecific antibody (Q460-z121/J327-z119/L334-k), wherein the first polypeptide is an H chain consisting of the amino acid sequence of SEQ ID NO: 18, the second polypeptide is an H chain consisting of the amino acid sequence of SEQ ID NO: 25, and the third polypeptide and the fourth polypeptide are a commonly shared L chain of SEQ ID NO: 30;
   (p) a bispecific antibody (Q499-z118/J327-z107/L334-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 30;
   (q) a bispecific antibody (Q499-z118/J327-z107/L377-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 31;
   (r) a bispecific antibody (Q499-z118/J346-z107/L248-k), wherein the first polypeptide is an H chain consisting of the amino acid sequence of SEQ ID NO: 19, the second polypeptide is an H chain consisting of the amino acid sequence of SEQ ID NO: 27, and the third polypeptide and the fourth polypeptide are a commonly shared L chain of SEQ ID NO: 28;
   (s) a bispecific antibody (Q499-z121/J327-z119/L404-k), wherein the first polypeptide is an H chain consisting of the amino acid sequence of SEQ ID NO: 20, the second polypeptide is an H chain consisting of the amino acid sequence of SEQ ID NO: 25, and the third polypeptide and the fourth polypeptide are a commonly shared L chain of SEQ ID NO: 32;
   (t) a bispecific antibody (Q499-z121/J339-z119/L377-k), wherein the first polypeptide is an H chain consisting of the amino acid sequence of SEQ ID NO: 20, the second polypeptide is an H chain consisting of the amino acid sequence of SEQ ID NO: 26, and the third polypeptide and the fourth polypeptide are a commonly shared L chain of SEQ ID NO: 31; and
   (u) a bispecific antibody (Q153-G4k/J142-G4h/L180-k), wherein the first polypeptide is an H chain consisting of the amino acid sequence of SEQ ID NO: 12, the second polypeptide is an H chain consisting of the amino acid sequence of SEQ ID NO: 170, and the third polypeptide and the fourth polypeptide are a commonly shared L chain of SEQ ID NO: 171;
[33] a nucleic acid encoding the bispecific antibody of [32];
[34] a vector inserted with the nucleic acid of [33];
[35] a cell comprising the nucleic acid of [33] or the vector of [34];
[36] a method for producing the bispecific antibody of [32] by culturing the cell of [35];
[37] a pharmaceutical composition comprising the bispecific antibody of [32], and a pharmaceutically acceptable carrier;
[38] the composition of [37], which is a pharmaceutical composition used for prevention and/or treatment of bleeding, a disease accompanying bleeding, or a disease caused by bleeding;
[39] the composition of [38], wherein the bleeding, the disease accompanying bleeding, or the disease caused by bleeding is a disease that develops and/or progresses due to a decrease or deficiency in the activity of blood coagulation factor VIII and/or activated blood coagulation factor VIII;
[40] the composition of [39], wherein the disease that develops and/or progresses due to a decrease or deficiency in the activity of blood coagulation factor VIII and/or activated blood coagulation factor VIII is hemophilia A;
[41] the composition of [39], wherein the disease that develops and/or progresses due to a decrease or deficiency in the activity of blood coagulation factor VIII and/or activated blood coagulation factor VIII is a disease showing emergence of an inhibitor against blood coagulation factor VIII and/or activated blood coagulation factor VIII;
[42] the composition of [39], wherein the disease that develops and/or progresses due to a decrease or deficiency in the activity of blood coagulation factor VIII and/or activated blood coagulation factor VIII is acquired hemophilia;
[43] the composition of [39], wherein the disease that develops and/or progresses due to a decrease in the activity of blood coagulation factor VIII and/or activated blood coagulation factor VIII is von Willebrand disease;
[44] a method for preventing and/or treating bleeding, a disease accompanying bleeding, or a disease caused by bleeding, which comprises the step of administering the bispecific antibody of [32] or the composition of any one of [37] to [43];
[45] a kit for use in the prevention and/or treatment method of [44], which comprises the bispecific antibody of [32], or the composition of any one of [37] to [43];
[46] use of the bispecific antibody of [32] or the composition of any one of [37] to [43] in the manufacture of an agent for preventing and/or treating bleeding, a disease accompanying bleeding, or a disease caused by bleeding; and
[47] the bispecific antibody of [32] or the composition of any one of [37] to [43] for preventing and/or treating bleeding, a disease accompanying bleeding, or a disease caused by bleeding.

### Effects of the Invention

The present invention provides antibodies that recognize both an enzyme and its substrate, which are multispecific antigen-binding molecules having a high activity of functionally substituting for F.VIII. Furthermore, the present invention provides antibodies that recognize both an enzyme and its substrate, which are multispecific antigen-binding molecules having a high activity of functionally substituting for F.VIII and a low F.Xase inhibitory action. Since humanized antibodies are generally thought to have high stability in blood and low immunogenicity, multispecific antibodies of the present invention may be very promising as pharmaceuticals.

### BRIEF DESCRIPTION OF THE DRAWINGS

FIG. 1 describes the F.Xase inhibitory action.
(a) F.VIIIa forms a complex with F.IXa (F.Xase) and activates F.X.
(b) A bispecific antibody binds to F.IXa and F.X and activates F.X.
(c) Both F.VIIIa and the bispecific antibody activate F.X without competition.
(d) Binding of the bispecific antibody to F.IXa and/or F.X inhibits the formation of the complex formed between F.Xase and F.X.
(e) Binding of the bispecific antibody to F.IXa and/or F.X inhibits the activity of F.Xase.

FIG. 2 describes the screening. Approximately 200 types each of genes for antibodies against human F.IXa and human F.X were produced, and they were incorporated into animal cell expression vectors. 40,000 or more bispecific antibodies as a combination of an anti-F.IXa antibody and anti-F.X antibody were transiently expressed. F.Xa generation-promoting activity and F.Xase inhibitory action were evaluated to screen for bispecific antibodies having a high F.Xa generation-promoting activity and a low F.Xase inhibitory action. Furthermore, by substituting amino acids when necessary, prototype antibodies were produced.

FIG. 3 shows the F.Xa generation-promoting activities of hA69-KQ/hB26-PF/hAL-AQ, Q1-G4k/J268-G4h/L45-k, Q1-G4k/J321-G4h/L45-k, Q31-z7/J326-z107/L2-k, and Q64-z55/J344-z107/L45-k. The concentrations of the antibody solutions were 300, 30, and 3 μg/mL (the concentrations after mixing Human Factor IXa, Novact (registered trademark) M, Human Factor X, and the antibody solution were 100, 10, and 1 μg/mL), the enzyme reaction and color development were performed for ten minutes and 50 minutes, respectively. As a result, these antibodies showed a higher F.Xa generation-promoting activity compared to hA69-KQ/hB26-PF/hAL-AQ described in WO 2006/109592.

FIG. 4 shows the F.Xa generation-promoting activity of hA69-KQ/hB26-PF/hAL-AQ, prototype antibodies, and modified antibodies with amino acid substitutions. The concentrations of the antibody solutions were 300, 30, and 3 μg/mL (the concentrations after mixing Human Factor IXa, Novact (registered trademark) M, Human Factor X, and the antibody solution were 100, 10, and 1 μg/mL), the enzyme reaction and color development were performed for two minutes and 20 minutes, respectively. As a result, these modified antibodies showed a higher F.Xa generation-promoting activity compared to the prototype antibodies.

FIG. 5 shows the F.Xase inhibitory action of hA69-KQ/hB26-PF/hAL-AQ, prototype antibodies, and modified antibodies with amino acid substitutions.

The figure shows the effects of hA69-KQ/hB26-PF/hAL-AQ, Q1-G4k/J268-G4h/L45-k, Q31-z7/J326-z107/L2-k, Q1-G4k/J321-G4h/L45-k, Q64-z55/J344-z107/L45-k, Q85-G4k/J268-G4h/L406-k, Q85-G4k/J321-G4h/L334-k, Q64-z7/J344-z107/L406-k, Q64-z7/J326-z107/L334-k, Q153-G4k/JT42-G4h/L180-k, Q405-G4k/J232-G4h/L248-k, Q360-G4k/J232-G4h/L406-k, Q153-G4k/J232-G4h/L406-k, Q458-z106/J346-z107/L408-k, Q360-z118/J300-z107/L334-k, Q499-z118/J327-z107/L377-k, Q499-z121/J327-z119/L404-k, Q499-z121/J339-z119/L377-k, Q499-z118/J346-z107/L248-k, Q354-z106/J259-z107/L324-k, Q460-z121/J327-z119/L334-k, and Q499-z118/J327-z107/L334-k on F.X activation by F.IXa in the presence of F.VIIIa. The F.Xase inhibitory actions of the antibodies are indicated as the value obtained by subtracting the absorbance of the antibody-free reaction solution from the absorbance of the antibody-supplemented reaction solution. The concentrations of the antibody solutions were 300 and 30 μg/mL (the concentrations after mixing Human Factor IXa, F.VIIIa, Human Factor X, and the antibody solution were 100 and 10 μg/mL), the enzyme reaction and color development were performed for six minutes and 14 minutes, respectively. The more positive the value of F.Xase inhibitory action shown on the horizontal axis, the weaker the F.Xase inhibitory action is. As a result, hA69-KQ/hB26-PF/hAL-AQ described in WO 2006/109592 showed strong F.Xase inhibitory action. All of the antibodies of the present invention showed weaker F.Xase inhibitory action compared to hA69-KQ/hB26-PF/hAL-AQ, or did not show inhibitory action.

FIG. 6A shows the amino acid sequences of the prototype antibodies and the modified antibodies with amino acid substitutions. When the sequence name is not indicated in the Ref column, the variable region sequence of the Name column is mentioned. A “- (hyphen)” is shown where an amino acid is absent at the number by Kabat numbering. A “. (dot)” is shown where amino acid is the same when comparing the variable region of the Name column and the Ref column, and the amino acid of the variable region of the Name column is shown where the amino acids are different. Amino acids found to be important for improvement of F.Xa generation-promoting activity were indicated by framing them.

FIG. 6B is a continuation of FIG. 6A.

FIG. 6C is a continuation of FIG. 6B.

FIG. 6D is a continuation of FIG. 6C.

The amino acid sequences shown in FIGS. 6A through 6D are Q1 (SEQ ID NO: 35), Q31 (SEQ ID NO: 36), Q64 (SEQ ID NO: 37), Q85 (SEQ ID NO: 38), Q153 (SEQ ID NO: 39), Q354 (SEQ ID NO: 40), Q360 (SEQ ID NO: 41), Q405 (SEQ ID NO: 42), Q458 (SEQ ID NO: 43), Q460 (SEQ ID NO: 44), Q499 (SEQ ID NO: 45), J268 (SEQ ID NO: 48), J321 (SEQ ID NO: 50), J326 (SEQ ID NO: 51), J344 (SEQ ID NO: 54), J232 (SEQ ID NO: 46), J259 (SEQ ID NO: 47), J346 (SEQ ID NO: 55), J300 (SEQ ID NO: 49), J327 (SEQ ID NO: 52), J339 (SEQ ID NO: 53), J142 (SEQ ID NO: 172), L2 (SEQ ID NO: 56), L45 (SEQ ID NO: 57), L248 (SEQ ID NO: 58), L324 (SEQ ID NO: 59), L334 (SEQ ID NO: 60), L377 (SEQ ID NO: 61), L404 (SEQ ID NO: 62), L406 (SEQ ID NO: 63), L408 (SEQ ID NO: 64), and L180 (SEQ ID NO: 173).

### MODE FOR CARRYING OUT THE INVENTION

Multispecific antigen-binding molecules described herein comprise a first antigen-binding site and a second antigen-binding site that can specifically bind to at least two different types of antigens. While the first antigen-binding site and the second antigen-binding site are not particularly limited as long as they have an activity to bind to F.IX and/or F.IXa, and F.X, respectively, examples include sites necessary for binding with antigens, such as antibodies, scaffold molecules (antibody-like molecules) or peptides, or fragments containing such sites. Scaffold molecules are molecules that exhibit function by binding to target molecules, and any polypeptide may be used as long as they are conformationally stable polypeptides that can bind to at least one target antigen. Examples of such polypeptides include antibody variable regions, fibronectin (WO 2002/032925), protein A domain (WO 1995/001937), LDL receptor A domain (WO 2004/044011, WO 2005/040229), ankyrin (WO 2002/020565), and such, and also molecules described in documents by Nygren et al. (Current Opinion in Structural Biology, 7: 463-469 (1997); and Journal of Immunol Methods, 290: 3-28 (2004)), Binz et al. (Nature Biotech 23: 1257-1266 (2005)), and Hosse et al. (Protein Science 15: 14-27(2006)). Furthermore, as mentioned in Curr Opin Mol Ther. 2010 August; 12(4): 487-95 and Drugs. 2008; 68(7): 901-12, peptide molecules that can bind to target antigens may be used.

Herein, multispecific antigen-binding molecules are not particularly limited as long as they are molecules that can bind to at least two different types of antigens, but examples include polypeptides containing the above-mentioned antigen-binding sites, such as antibodies and scaffold molecules as well as their fragments, and aptamers comprising nucleic acid molecules and peptides, and they may be single molecules or multimers thereof. Preferred multispecific antigen-binding molecules include multispecific antibodies that can bind specifically to at least two different antigens. Particularly preferred examples of antibodies which have an activity of functionally substituting for F.VIII of the present invention include bispecific antibodies (BsAb) that can bind specifically to two different antigens (they may also be called dual specific antibodies).

In the present invention, the term “commonly shared L chain” refers to an L chain that can link with two or more different H chains, and show binding ability to each antigen. Herein, the term “different H chain(s)” preferably refers to H chains of antibodies against different antigens, but is not limited thereto, and also refers to H chains whose amino acid sequences are different from each other. Commonly shared L chain can be obtained, for example, according to the method described in WO 2006/109592.

The multispecific antigen-binding molecules of the present invention (preferably bispecific antibodies) are antibodies having specificity to two or more different antigens, or molecules comprising fragments of such antibodies. The antibodies of the present invention are not particularly limited, but are preferably monoclonal antibodies. Monoclonal antibodies used in the present invention include not only monoclonal antibodies derived from animals such as humans, mice, rats, hamsters, rabbits, sheep, camels, and monkeys, but also include artificially modified gene recombinant antibodies such as chimeric antibodies, humanized antibodies, and bispecific antibodies.

Furthermore, the L chains of an antibody which will become a multispecific antigen-binding molecule of the present invention may be different, but preferably have commonly shared L chains.

Multispecific antigen-binding molecules of the present invention are preferably recombinant antibodies produced using genetic recombination techniques (See, for example, Borrebaeck C A K and Larrick J W, THERAPEUTIC MONOCLONAL ANTIBODIES, Published in the United Kingdom by MACMILLAN PUBLISHERS LTD, 1990). Recombinant antibodies can be obtained by cloning DNAs encoding antibodies from hybridomas or antibody-producing cells, such as sensitized lymphocytes, that produce antibodies, inserting them into suitable vectors, and then introducing them into hosts (host cells) to produce the antibodies.

Furthermore, antibodies of the present invention may include not only whole antibodies but also antibody fragments and low-molecular-weight antibodies (minibodies), and modified antibodies.

For example, antibody fragments or minibodies include diabodies (Dbs), linear antibodies, and single chain antibody (hereinafter, also denoted as scFvs) molecules. Herein, an “Fv” fragment is defined as the smallest antibody fragment that comprises a complete antigen recognition site and binding site.

An “Fv” fragment is a dimer (VH-VL dimer) in which an H chain variable region (VH) and an L chain variable region (VL) are strongly linked by non-covalent binding. The three complementarity determining regions (CDRs) of each of the variable regions interact with each other to form an antigen-binding site on the surface of the VH-VL dimer. Six CDRs confer the antigen-binding site to an antibody. However, one variable region (or half of the Fv comprising only three CDRs specific to an antigen) alone can recognize and bind to an antigen, though its affinity is lower than that of the entire binding site.

An Fab fragment (also called F(ab)) further comprises an L chain constant region and an H chain constant region (CHI). An Fab′ fragment differs from an Fab fragment in that it additionally comprises several residues derived from the carboxyl terminus of the H chain CHI region, comprising one or more cysteines from the hinge region of the antibody. Fab′-SH refers to an Fab′ in which one or more cysteine residues of its constant region comprise a free thiol group. An F(ab′) fragment is produced by cleavage of disulfide bonds between the cysteine residues in the hinge region of F(ab′)₂ pepsin digest. Other chemically bound antibody fragments are also known to those skilled in the art.

Diabodies are bivalent minibodies constructed by gene fusion (Holliger, P. et al., Proc. Natl. Acad. Sci. USA 90: 6444-6448 (1993); EP 404,097; WO 93/11161). Diabodies are dimers consisting of two polypeptide chains, in which each polypeptide chain comprises an L chain variable region (VL) and an H chain variable region (VH) linked with a linker short enough to prevent association of these two domains within the same chain, for example, a linker of preferably 2 to 12 amino acids, more preferably 3 to 10 amino acids, particularly about 5 amino acids. The polypeptide chain form a dimer since the linker between the VL and VH encoded on the same polypeptide is too short to form a single chain variable region fragment. Therefore, diabodies comprise two antigen-binding sites.

A single-chain antibody or an scFv antibody fragment comprises the VH and VL regions of an antibody, and these regions exist in a single polypeptide chain. In general, an Fv polypeptide further comprises a polypeptide linker between the VH and VL regions, and this enables an scFv to form a structure necessary for antigen binding (for a review on scFvs, see Pluckthun “The Pharmacology of Monoclonal Antibodies” Vol. 113 (Rosenburg and Moore ed. (Springer Verlag, New York) pp. 269-315, 1994). In the context of the present invention, linkers are not particularly limited so long as they do not inhibit the expression of the antibody variable regions linked at their ends.

IgG-type bispecific antibodies can be secreted from hybrid hybridomas (quadromas) produced by fusing two kinds of hybridomas that produce IgG antibodies (Milstein C et al. Nature 1983, 305: 537-540). They can also be secreted by taking the L chain and H chain genes constituting the two kinds of IgGs of interest, a total of four kinds of genes, and introducing them into cells to coexpress the genes.

In this case, by introducing suitable amino acid substitutions to the CH3 regions of the H chains, IgGs having a heterogeneous combination of H chains can be preferentially secreted (Ridgway J B et al. Protein Engineering 1996, 9: 617-621; Merchant A M et al. Nature Biotechnology 1998, 16: 677-681; WO 2006/106905; Davis J H et al. Protein Eng Des Sel. 2010, 4: 195-202).

Regarding the L chains, since diversity of L chain variable regions is lower than that of H chain variable regions, commonly shared L chains that can confer binding ability to both H chains may be obtained. The antibodies of the present invention comprise commonly shared L chains. Bispecific IgGs can be efficiently expressed by introducing the genes of the commonly shared L chain and both H chains into cells.

Bispecific antibodies may be produced by chemically crosslinking Fab's. Bispecific F(ab′)₂ can be produced, for example, by preparing Fab′ from an antibody, using it to produce a maleimidized Fab′ with ortho-phenylenedi-maleimide (o-PDM), and then reacting this with Fab′ prepared from another antibody to crosslink Fab's derived from different antibodies (Keler T et al. Cancer Research 1997, 57: 4008-4014). The method of chemically linking an Fab′-thionitrobenzoic acid (TNB) derivative and an antibody fragment such as Fab′-thiol (SH) is also known (Brennan M et al. Science 1985, 229: 81-83).

Instead of a chemical crosslink, a leucine zipper derived from Fos and Jun may also be used. Preferential formation of heterodimers by Fos and Jun is utilized, even though they also form homodimers. Fab′ to which Fos leucine zipper is added, and another Fab′ to which Jun leucine zipper is added are expressed and prepared. Monomeric Fab′-Fos and Fab′-Jun reduced under mild conditions are mixed and reacted to form bispecific F(ab′)₂ (Kostelny S A et al. J. of Immunology, 1992, 148: 1547-53). This method can be applied not only to Fab's but also to scFvs, Fvs, and such.

Furthermore, bispecific antibodies including sc(Fv)₂ such as IgG-scFv (Protein Eng Des Sel. 2010 April; 23(4): 221-8) and BiTE (Drug Discov Today. 2005 Sep. 15; 10(18): 1237-44.), DVD-Ig (Nat Biotechnol. 2007 November; 25(11): 1290-7. Epub 2007 Oct. 14.; and MAbs. 2009 July; 1(4): 339-47. Epub 2009 Jul. 10.), and also others (IDrugs 2010, 13: 698-700) including two-in-one antibodies (Science. 2009 Mar. 20; 323(5921): 1610-4; and Immunotherapy. 2009 September; 1(5): 749-51.), Tri-Fab, tandem scFv, and diabodies are known (MAbs. 2009 November; 1(6): 539-547). In addition, even when using molecular forms such as scFv-Fc and scaffold-Fc, bispecific antibodies can be produced efficiently by preferentially secreting a heterologous combination of Fcs (Ridgway J B et al., Protein Engineering 1996, 9: 617-621; Merchant A M et al. Nature Biotechnology 1998, 16: 677-681; WO 2006/106905; and Davis J H et al., Protein Eng Des Sel. 2010, 4: 195-202.).

A bispecific antibody may also be produced using a diabody. A bispecific diabody is a heterodimer of two cross-over scFv fragments. More specifically, it is produced by forming a heterodimer using VH(A)-VL(B) and VH(B)-VL(A) prepared by linking VHs and VLs derived from two kinds of antibodies, A and B, using a relatively short linker of about 5 residues (Holliger P et al. Proc Natl. Acad. Sci. USA 1993, 90: 6444-6448).

The desired structure can be achieved by linking the two scFvs with a flexible and relatively long linker comprising about 15 residues (single chain diabody: Kipriyanov S M et al. J. of Molecular Biology. 1999, 293: 41-56), and conducting appropriate amino acid substitutions (knobs-into-holes: Zhu Z et al. Protein Science. 1997, 6: 781-788; VH/VL interface engineering: Igawa T et al. Protein Eng Des Sel. 2010, 8: 667-77).

An sc(Fv)₂ that can be produced by linking two types of scFvs with a flexible and relatively long linker, comprising about 15 residues, may also be a bispecific antibody (Mallender W D et al. J. of Biological Chemistry, 1994, 269: 199-206).

Examples of modified antibodies include antibodies linked to various molecules such as polyethylene glycol (PEG). The antibodies of the present invention include such modified antibodies. In the context of the present invention, the substance to which the modified antibodies are linked is not limited. Such modified antibodies can be obtained by chemically modifying obtained antibodies. Such methods are well established in the art.

The antibodies of the present invention include human antibodies, mouse antibodies, rat antibodies, or such, and their origins are not limited. They may also be genetically modified antibodies, such as chimeric or humanized antibodies.

Methods for obtaining human antibodies are known in the art. For example, transgenic animals carrying the entire repertoire of human antibody genes can be immunized with desired antigens to obtain desired human antibodies (see International Patent Application WO 93/12227, WO 92/03918, WO 94/02602, WO 94/25585, WO 96/34096, and WO 96/33735).

Genetically modified antibodies can also be produced using known methods. Specifically, for example, chimeric antibodies may comprise H chain and L chain variable regions of an immunized animal antibody, and H chain and L chain constant regions of a human antibody. Chimeric antibodies can be obtained by linking DNAs encoding the variable regions of the antibody derived from the immunized animal, with DNAs encoding the constant regions of a human antibody, inserting this into an expression vector, and then introducing it into host cells to produce the antibodies.

Humanized antibodies are modified antibodies often referred to as “reshaped” human antibodies. A humanized antibody is constructed by transferring the CDRs of an antibody derived from an immunized animal to the complementarity determining regions of a human antibody. Conventional genetic recombination techniques for such purposes are known (see European Patent Application Publication No. EP 239400; International Publication No. WO 96/02576; Sato K et al., Cancer Research 1993, 53: 851-856; International Publication No. WO 99/51743).

The multispecific antigen-binding molecules of the present invention are those that recognize F.IX and/or F.IXa, and F.X, and functionally substitute for cofactor function of F.VIII, and characterized in that the molecules have a higher F.Xa generation-promoting activity compared to hA69-KQ/hB26-PF/hAL-AQ (described in WO 2006/109592) which is known as a bispecific antibody that functionally substitutes for F.VIII. Furthermore, antibodies of the present invention usually have a structure which comprises a variable region of an anti-F.IXa antibody and a variable region of an anti-F.X antibody.

More specifically, the present invention provides a multispecific antigen-binding molecule that functionally substitutes for F.VIII, which comprises a first antigen-binding site that recognizes F.IX and/or F.IXa and a second antigen-binding site that recognizes F.X, wherein the function that substitutes for the function of F.VIII is caused by a higher F.Xa generation-promoting activity compared to the activity of the bispecific antibody (hA69-KQ/hB26-PF/hAL-AQ) which comprises H chains consisting of SEQ ID NOs: 165 and 166, and a commonly shared L chain consisting of SEQ ID NO: 167.

A multispecific antigen-binding molecule of the present invention comprises a first polypeptide and a third polypeptide comprising an antigen-binding site that recognizes F.IX and/or F.IXa, and a second polypeptide and a fourth polypeptide comprising an antigen-binding site that recognizes F.X. The first polypeptide and the third polypeptide, and the second polypeptide and the fourth polypeptide each include the antigen-binding site of the antibody H chain and the antigen-binding site of the antibody L chain.

For example, in a multispecific antigen-binding molecule of the present invention, the first polypeptide and the third polypeptide include an antigen-binding site of an H chain and L chain of an antibody against F.IX or F.IXa, respectively; and the second polypeptide and the fourth polypeptide comprise an antigen-binding site of an H chain and L chain of an antibody against F.X, respectively.

At this time, the antigen-binding sites of the antibody L chain included in the first polypeptide and the third polypeptide, and the second polypeptide and the fourth polypeptide may be commonly shared L chains.

A polypeptide comprising an antigen-binding site of an antibody L chain in the present invention is preferably a polypeptide which comprises all or a part of the sequence of the antibody L chain which binds to F.IX, F.IXa and/or F.X.

Preferred embodiments of the antigen-binding site of the first polypeptide of an antibody of the present invention specifically include antigen-binding sites comprising the amino acid sequences of:
Q1 H chain each CDR1, 2, and 3 sequence (SEQ ID NOs: 75, 76, and 77, respectively);
Q31 H chain each CDR1, 2, and 3 sequences (SEQ ID NOs: 78, 79, and 80, respectively);
Q64 H chain each CDR1, 2, and 3 sequence (SEQ ID NOs: 81, 82, and 83, respectively);
Q85 H chain each CDR1, 2, and 3 sequence (SEQ ID NOs: 84, 85, and 86, respectively);
Q153 H chain each CDR1, 2, and 3 sequence (SEQ ID NOs: 87, 88, and 89, respectively);
Q354 H chain each CDR1, 2, and 3 sequence (SEQ ID NOs: 90, 91, and 92, respectively);
Q360 H chain each CDR1, 2, and 3 sequence (SEQ ID NOs: 93, 94, and 95, respectively);
Q405 H chain each CDR1, 2, and 3 sequence (SEQ ID NOs: 96, 97, and 98, respectively);
Q458 H chain each CDR1, 2, and 3 sequence (SEQ ID NOs: 99, 100, and 101, respectively);
Q460 H chain each CDR1, 2, and 3 sequence (SEQ ID NOs: 102, 103, and 104, respectively); and
Q499 H chain each CDR1, 2, and 3 sequence (SEQ ID NOs: 105, 106, and 107, respectively) mentioned in the later-described Examples, or antigen-binding sites that are functionally equivalent to them.

Preferred embodiments of the antigen-binding site of a second polypeptide specifically include, for example, antigen-binding sites comprising the amino acid sequences of:
J232 H chain each CDR1, 2, and 3 sequence (SEQ ID NOs: 108, 109, and 110, respectively);
J259 H chain each CDR1, 2, and 3 sequence (SEQ ID NOs: 111, 112, and 113, respectively);
J268 H chain each CDR1, 2, and 3 sequence (SEQ ID NOs: 114, 115, and 116, respectively);
J300 H chain each CDR1, 2, and 3 sequence (SEQ ID NOs: 117, 118, and 119, respectively);
J321 H chain each CDR1, 2, and 3 sequence (SEQ ID NOs: 120, 121, and 122, respectively);
J326 H chain each CDR1, 2, and 3 sequence (SEQ ID NOs: 123, 124, and 125, respectively);
J327 H chain each CDR1, 2, and 3 sequence (SEQ ID NOs: 126, 127, and 128, respectively);
J339 H chain each CDR1, 2, and 3 sequence (SEQ ID NOs: 129, 130, and 131, respectively);
J344 H chain each CDR1, 2, and 3 sequence (SEQ ID NOs: 132, 133, and 134, respectively);
J346 H chain each CDR1, 2, and 3 sequence (SEQ ID NOs: 135, 136, and 137, respectively); and
J142 H chain each CDR1, 2, and 3 sequence (SEQ ID NOs: 174, 175, and 176, respectively) mentioned in the later-described Examples, or antigen-binding sites that are functionally equivalent to them.

More specifically, the present invention provides multispecific antigen-binding molecules, wherein the antigen-binding site of the first polypeptide comprises an antigen-binding site which comprises H chain CDRs consisting of any one of the amino acid sequences selected from the following (a1) to (a11), or an antigen-binding site functionally equivalent thereto, and the antigen-binding site of the second polypeptide comprises an antigen-binding site which comprises H chain CDRs consisting of any one of the amino acid sequences selected from the following (b1) to (b11), or an antigen-binding site functionally equivalent thereto:
(a1) an antigen-binding site comprising an H chain CDR 1, 2, and 3 amino acid sequences of SEQ ID NOs: 75, 76, and 77 (H chain CDRs of Q1), respectively;
(a2) an antigen-binding site comprising an H chain CDR 1, 2, and 3 amino acid sequences of SEQ ID NOs: 78, 79, and 80 (H chain CDRs of Q31), respectively;
(a3) an antigen-binding site comprising an H chain CDR 1, 2, and 3 amino acid sequences of SEQ ID NOs: 81, 82, and 83 (H chain CDRs of Q64), respectively;
(a4) an antigen-binding site comprising an H chain CDR 1, 2, and 3 amino acid sequences of SEQ ID NOs: 84, 85, and 86 (H chain CDRs of Q85), respectively;
(a5) an antigen-binding site comprising the H chain CDR 1, 2, and 3 amino acid sequences of SEQ ID NOs: 87, 88, and 89 (H chain CDRs of Q153), respectively;
(a6) an antigen-binding site comprising an H chain CDR 1, 2, and 3 amino acid sequences of SEQ ID NOs: 90, 91, and 92 (H chain CDRs of Q354), respectively;
(a7) an antigen-binding site comprising the H chain CDR 1, 2, and 3 amino acid sequences of SEQ ID NOs: 93, 94, and 95 (H chain CDRs of Q360), respectively;
(a8) an antigen-binding site comprising the of H chain CDR 1, 2, and 3 amino acid sequences of SEQ ID NOs: 96, 97, and 98 (H chain CDRs of Q405), respectively;
(a9) an antigen-binding site comprising an H chain CDR 1, 2, and 3 amino acid sequences of SEQ ID NOs: 99, 100, and 101 (H chain CDRs of Q458), respectively;
(a10) an antigen-binding site comprising an H chain CDR 1, 2, and 3 amino acid sequences of SEQ ID NOs: 102, 103, and 104 (H chain CDRs of Q460), respectively;
(a11) an antigen-binding site comprising an H chain CDR 1, 2, and 3 amino acid sequences of SEQ ID NOs: 105, 106, and 107 (H chain CDRs of Q499), respectively;
(b1) an antigen-binding site comprising an H chain CDR 1, 2, and 3 amino acid sequences of SEQ ID NOs: 108, 109, and 110 (H chain CDRs of J232), respectively;
(b2) an antigen-binding site comprising an H chain CDR 1, 2, and 3 amino acid sequences of SEQ ID NOs: 111, 112, and 113 (H chain CDRs of J259), respectively;
(b3) an antigen-binding site comprising an H chain CDR 1, 2, and 3 amino acid sequences of SEQ ID NOs: 114, 115, and 116 (H chain CDRs of J268), respectively;
(b4) an antigen-binding site comprising an H chain CDR 1, 2, and 3 amino acid sequences of SEQ ID NOs: 117, 118, and 119 (H chain CDRs of J300), respectively;
(b5) an antigen-binding site comprising an H chain CDR 1, 2, and 3 amino acid sequences of SEQ ID NOs: 120, 121, and 122 (H chain CDRs of J321), respectively;
(b6) an antigen-binding site comprising the H chain CDR 1, 2, and 3 amino acid sequences of SEQ ID NOs: 123, 124, and 125 (H chain CDRs of J326), respectively;
(b7) an antigen-binding site comprising an H chain CDR 1, 2, and 3 amino acid sequences of SEQ ID NOs: 126, 127, and 128 (H chain CDRs of J327), respectively;
(b8) an antigen-binding site comprising an H chain CDR 1, 2, and 3 amino acid sequences of SEQ ID NOs: 129, 130, and 131 (H chain CDRs of J339), respectively;
(b9) an antigen-binding site comprising an H chain CDR 1, 2, and 3 amino acid sequences of SEQ ID NOs: 132, 133, and 134 (H chain CDRs of J344), respectively;
(b10) an antigen-binding site comprising an H chain CDR 1, 2, and 3 amino acid sequences of SEQ ID NOs: 135, 136, and 137 (H chain CDRs of J346), respectively; and
(b11) an antigen-binding site comprising an H chain CDR 1, 2, and 3 amino acid sequences of SEQ ID NOs: 174, 175, and 176 (H chain CDRs of J142), respectively.

Preferred embodiments of the antigen-binding site of the third and fourth polypeptides specifically include, for example, antigen-binding sites comprising the amino acid sequences of:
L2 L chain each CDR1, 2, and 3 sequence (SEQ ID NOs: 138, 139, and 140, respectively);
L45 L chain each CDR1, 2, and 3 sequence (SEQ ID NOs: 141, 142, and 143, respectively);
L248 L chain each CDR1, 2, and 3 sequence (SEQ ID NOs: 144, 145, and 146, respectively);
L324 L chain each CDR1, 2, and 3 sequence (SEQ ID NOs: 147, 148, and 149, respectively);
L334 L chain each CDR1, 2, and 3 sequence (SEQ ID NOs: 150, 151, and 152, respectively);
L377 L chain each CDR1, 2, and 3 sequence (SEQ ID NOs: 153, 154, and 155, respectively);
L404 L chain each CDR1, 2, and 3 sequence (SEQ ID NOs: 156, 157, and 158, respectively);
L406 L chain each CDR1, 2, and 3 sequence (SEQ ID NOs: 159, 160, and 161, respectively);
L408 L chain each CDR1, 2, and 3 sequence (SEQ ID NOs: 162, 163, and 164, respectively); and
L180 L chain each CDR1, 2, and 3 sequence (SEQ ID NOs: 177, 178, and 179, respectively) mentioned in the later-described Examples, or antigen-binding sites that are functionally equivalent to them.

More specifically, the present invention provides multispecific antigen-binding molecules, wherein the antigen-binding sites included in the third polypeptide and the fourth polypeptide comprise an antigen-binding site which comprises L chain CDRs consisting of any one of the amino acid sequences selected from the following (c1) to (c10), or an antigen-binding site functionally equivalent thereto:
(c1) an antigen-binding site comprising an L chain CDR1, 2, and 3 amino acid sequences of SEQ ID NOs: 138, 139, and 140 (L chain CDR of L2), respectively;
(c2) an antigen-binding site comprising an L chain CDR1, 2, and 3 amino acid sequences of SEQ ID NOs: 141, 142, and 143 (L chain CDR of L45), respectively;
(c3) an antigen-binding site comprising an L chain CDR1, 2, and 3 amino acid sequences of SEQ ID NOs: 144, 145, and 146 (L chain CDR of L248), respectively;
(c4) an antigen-binding site comprising an L chain CDR1, 2, and 3 amino acid sequences of SEQ ID NOs: 147, 148, and 149 (L chain CDR of L324), respectively;
(c5) an antigen-binding site comprising an L chain CDR1, 2, and 3 amino acid sequences of SEQ ID NOs: 150, 151, and 152 (L chain CDR of L334), respectively;
(c6) an antigen-binding site comprising an L chain CDR1, 2, and 3 amino acid sequences of SEQ ID NOs: 153, 154, and 155 (L chain CDR of L377), respectively;
(c7) an antigen-binding site comprising an L chain CDR1, 2, and 3 amino acid sequences of SEQ ID NOs: 156, 157, and 158 (L chain CDR of L404), respectively;
(c8) an antigen-binding site comprising an L chain CDR1, 2, and 3 amino acid sequences of SEQ ID NOs: 159, 160, and 161 (L chain CDR of L406), respectively;
(c9) an antigen-binding site comprising an L chain CDR1, 2, and 3 amino acid sequences of SEQ ID NOs: 137, 138, and 139 (L chain CDR of L408), respectively; and
(c10) an antigen-binding site comprising an L chain CDR1, 2, and 3 amino acid sequences of SEQ ID NOs: 177, 178, and 179 (L chain CDR of L180), respectively.

The amino acid sequences of the H chain variable regions of Q1, Q31, Q64, Q85, Q153, Q354, Q360, Q405, Q458, Q460, and Q499 of the present invention are indicated by the following SEQ ID NOs, respectively.
Q1: SEQ ID NO: 35
Q31: SEQ ID NO: 36
Q64: SEQ ID NO: 37
Q85: SEQ ID NO: 38
Q153: SEQ ID NO: 39
Q354: SEQ ID NO: 40
Q360: SEQ ID NO: 41
Q405: SEQ ID NO: 42
Q458: SEQ ID NO: 43
Q460: SEQ ID NO: 44
Q499: SEQ ID NO: 45

The amino acid sequences of the H chain variable regions of J232, J259, J268, J300, J321, J326, J327, J339, J344, J346, and J142 of the present invention are indicated by the following SEQ ID NOs, respectively.
J232: SEQ ID NO: 46
J259: SEQ ID NO: 47
J268: SEQ ID NO: 48
J300: SEQ ID NO: 49
J321: SEQ ID NO: 50
J326: SEQ ID NO: 51
J327: SEQ ID NO: 52
J339: SEQ ID NO: 53
J344: SEQ ID NO: 54
J346: SEQ ID NO: 55
J142: SEQ ID NO: 172

More specifically, the present invention provides multispecific antigen-binding molecules, wherein the antigen-binding site of the first polypeptide comprises an antigen-binding site which comprises an H chain variable region consisting of any one of the amino acid sequences selected from the following (a1) to (a11), or an antigen-binding site functionally equivalent thereto, and the antigen-binding site of the second polypeptide comprises an antigen-binding site which comprises an H chain variable region consisting of any one of the amino acid sequences selected from the following (b1) to (b11), or an antigen-binding site functionally equivalent thereto:
(a1) an antigen-binding site comprising an H chain variable region amino acid sequence of SEQ ID NO: 35 (H chain variable region of Q1);
(a2) an antigen-binding site comprising an H chain variable region amino acid sequence of SEQ ID NO: 36 (H chain variable region of Q31);
(a3) an antigen-binding site comprising an H chain variable region amino acid sequence of SEQ ID NO: 37 (H chain variable region of Q64);
(a4) an antigen-binding site comprising an H chain variable region amino acid sequence of SEQ ID NO: 38 (H chain variable region of Q85);
(a5) an antigen-binding site comprising an H chain variable region amino acid sequence of SEQ ID NO: 39 (H chain variable region of Q153);
(a6) an antigen-binding site comprising an H chain variable region amino acid sequence of SEQ ID NO: 40 (H chain variable region of Q354);
(a7) an antigen-binding site comprising an H chain variable region amino acid sequence of SEQ ID NO: 41 (H chain variable region of Q360);
(a8) an antigen-binding site comprising an H chain variable region amino acid sequence of SEQ ID NO: 42 (H chain variable region of Q405);
(a9) an antigen-binding site comprising an H chain variable region amino acid sequence of SEQ ID NO: 43 (H chain variable region of Q458);
(a10) an antigen-binding site comprising an H chain variable region amino acid sequence of SEQ ID NO: 44 (H chain variable region of Q460);
(a11) an antigen-binding site comprising an H chain variable region amino acid sequence of SEQ ID NO: 45 (H chain variable region of Q499);
(b1) an antigen-binding site comprising an H chain variable region amino acid sequence of SEQ ID NO: 46 (H chain variable region of J232);
(b2) an antigen-binding site comprising an H chain variable region amino acid sequence of SEQ ID NO: 47 (H chain variable region of J259);
(b3) an antigen-binding site comprising an H chain variable region amino acid sequence of SEQ ID NO: 48 (H chain variable region of J268);
(b4) an antigen-binding site comprising an H chain variable region amino acid sequence of SEQ ID NO: 49 (H chain variable region of J300);
(b5) an antigen-binding site comprising an H chain variable region amino acid sequence of SEQ ID NO: 50 (H chain variable region of J321);
(b6) an antigen-binding site comprising an H chain variable region amino acid sequence of SEQ ID NO: 51 (H chain variable region of J326);
(b7) an antigen-binding site comprising an H chain variable region amino acid sequence of SEQ ID NO: 52 (H chain variable region of J327);
(b8) an antigen-binding site comprising an H chain variable region amino acid sequence of SEQ ID NO: 53 (H chain variable region of J339);
(b9) an antigen-binding site comprising an H chain variable region amino acid sequence of SEQ ID NO: 54 (H chain variable region of J344);
(b10) an antigen-binding site comprising an H chain variable region amino acid sequence of SEQ ID NO: 55 (H chain variable region of J346); and
(b11) an antigen-binding site comprising an H chain variable region amino acid sequence of SEQ ID NO: 172 (H chain variable region of J142).

In addition, the amino acid sequences of the L chain variable regions of L2, L45, L248, L324, L334, L377, L404, L406, L408, and L180 of the present invention are indicated by the following SEQ ID NOs, respectively.
L2: SEQ ID NO: 56
L45: SEQ ID NO: 57
L248: SEQ ID NO: 58
L324: SEQ ID NO: 59
L334: SEQ ID NO: 60
L377: SEQ ID NO: 61
L404: SEQ ID NO: 62
L406: SEQ ID NO: 63
L408: SEQ ID NO: 64
L180: SEQ ID NO: 173

More specifically, the present invention provides multispecific antigen-binding molecules, wherein the antigen-binding sites included in the third polypeptide and the fourth polypeptide comprise an antigen-binding site which comprises an L chain variable region consisting of any one of the amino acid sequences selected from the following (c1) to (c10), or an antigen-binding site functionally equivalent thereto:
(c1) an antigen-binding site comprising an L chain variable region amino acid sequence of SEQ ID NO: 56 (L chain variable region of L2);
(c2) an antigen-binding site comprising an L chain variable region amino acid sequence of SEQ ID NO: 57 (L chain variable region of L45);
(c3) an antigen-binding site comprising an L chain variable region amino acid sequence of SEQ ID NO: 58 (L chain variable region of L248);
(c4) an antigen-binding site comprising an L chain variable region amino acid sequence of SEQ ID NO: 59 (L chain variable region of L324);
(c5) an antigen-binding site comprising an L chain variable region amino acid sequence of SEQ ID NO: 60 (L chain variable region of L334);
(c6) an antigen-binding site comprising an L chain variable region amino acid sequence of SEQ ID NO: 61 (L chain variable region of L377);
(c7) an antigen-binding site comprising an L chain variable region amino acid sequence of SEQ ID NO: 62 (L chain variable region of L404);
(c8) an antigen-binding site comprising an L chain variable region amino acid sequence of SEQ ID NO: 63 (L chain variable region of L406);
(c9) an antigen-binding site comprising an L chain variable region amino acid sequence of SEQ ID NO: 64 (L chain variable region of L408); and
(c10) an antigen-binding site comprising an L chain variable region amino acid sequence of SEQ ID NO: 173 (L chain variable region of L180).

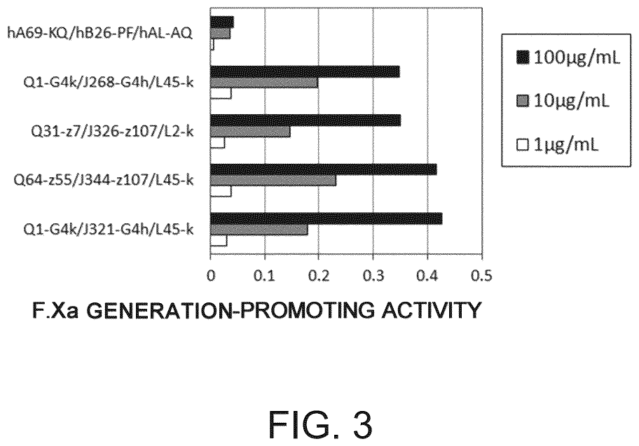

The amino acid sequences of CDR1 to 3 and FR1 to 4 in each of the sequences are as described in FIGS. 3A to D

When producing a full-length antibody using the variable regions disclosed in the present invention, without particular limitations, constant regions well known to those skilled in the art may be used. For example, constant regions described in “Sequences of proteins of immunological interest”, (1991), U.S. Department of Health and Human Services. Public Health Service National Institutes of Health, or “An efficient route to human bispecific IgG”, (1998). Nature Biotechnology vol. 16, 677-681 can be used. Preferred examples of the antibody constant regions of the present invention include the constant regions of IgG antibodies. When using the constant region of an IgG antibody, its type is not limited, and a constant region of IgG subclass such as IgG1, IgG2, IgG3, or IgG4 may be used. Furthermore, amino acid mutations may be introduced into the constant region of these IgG subclasses. Amino acid mutations to be introduced may be, for example, those that enhance or decrease binding to Fcγ receptors (Proc Natl Acad Sci USA. 2006 Mar. 14; 103(11): 4005-10; and MAbs. 2009 November; 1(6): 572-9), or enhance or decrease binding to FcRn (J Biol Chem. 2001 Mar. 2; 276(9): 6591-604; Int Immunol. 2006 Dec.; 18(12): 1759-69; and J Biol Chem. 2006 Aug. 18; 281(33): 23514-24), but are not limited thereto. Two types of H chains must be heterologously associated to produce a bispecific antibody. The knobs-into-holes technology (J Immunol Methods. 2001 Feb. 1; 248(1-2): 7-15; and J Biol Chem. 2010 Jul. 2; 285(27): 20850-9), the electrostatic repulsion technology (WO 2006/106905), the SEEDbody technology (Protein Eng Des Sel. 2010 April; 23(4): 195-202), and such may be used for heterologous association of two types of H chains via a CH3 domain. Furthermore, the antibodies of the present invention may be those with a modified or deficient sugar chain. Examples of antibodies having modified sugar chains include glycosylation-engineered antibodies (such as WO 99/54342), antibodies with defucosylated sugar chains (WO 00/61739, WO 02/31140, WO 2006/067847, WO 2006/067913, etc.), and antibodies having a sugar chain with bisecting GlcNAc (such as WO 02/79255). Known examples of methods for producing sugar chain-deficient IgG antibodies include the method of introducing a mutation to asparagine at position 297 in the EU numbering (J Clin Pharmacol. 2010 May; 50(5): 494-506), and the method of producing IgG using Escherichia coli (J Immunol Methods. 2002 May 1; 263(1-2): 133-47; and J Biol Chem. 2010 Jul. 2; 285(27): 20850-9). Furthermore, heterogeneity accompanying deletion of C-terminal lysine in IgG, and heterogeneity accompanying mispairing of disulfide bonds in the hinge region of IgG2 can be decreased by introducing amino acid deletions/substitutions (WO 2009/041613).

The present invention provides, for example, multispecific antigen-binding molecules, wherein the first and second polypeptides comprise an antibody H chain constant region, and the third and fourth polypeptides comprise an antibody L chain constant region.

Furthermore, the present invention provides multispecific antigen-binding molecules, wherein the first polypeptide comprises an antibody H chain constant region consisting of any one of the amino acid sequences selected from the group consisting of the following (d1) to (d6) or the group consisting of the following (d7) to (d9), and the second polypeptide comprises an antibody H chain constant region consisting of any one of the amino acid sequences selected from a group different from that of the above-mentioned first polypeptide:
(d1) an H chain constant region of SEQ ID NO: 65 (G4k);
(d2) an H chain constant region of SEQ ID NO: 66 (z7);
(d3) an H chain constant region of SEQ ID NO: 67 (z55);
(d4) an H chain constant region of SEQ ID NO: 68 (z106);
(d5) an H chain constant region of SEQ ID NO: 69 (z118);
(d6) an H chain constant region of SEQ ID NO: 70 (z121);
(d7) an H chain constant region of SEQ ID NO: 71 (G4h);
(d8) an H chain constant region of SEQ ID NO: 72 (z107); and
(d9) an H chain constant region of SEQ ID NO: 73 (z119).

Furthermore, the present invention provides a multispecific antigen-binding molecule, wherein the third and fourth polypeptides comprise an antibody L chain constant region consisting of the following amino acid sequence of:
(e) an L chain constant region of SEQ ID NO: 74 (k).

In the present invention, the phrase “functionally substitute for F.VIII” means that F.IX and/or F.IXa, and F.X is recognized, and activation of F.X is promoted (F.Xa generation is promoted).

In the present invention, “F.Xa generation-promoting activity” can be confirmed by evaluating the multispecific antigen-binding molecules of the present invention using, for example, a measurement system comprising F.XIa (F.IX activating enzyme), F.IX, F.X, F synthetic substrate S-2222 (synthetic substrate of F.Xa), and phospholipids. This measurement system shows the correlation between the severity of the disease and clinical symptoms in hemophilia A cases (Rosen S, Andersson M, Blomba\ddot{ }ck M et al. Clinical applications of a chromogenic substrate method for determination of FVIII activity. Thromb Haemost 1985, 54: 811-23). That is, in the present measurement system, test substances that show higher F.Xa generation-promoting activity are expected to show better hemostatic effects against bleeding episodes in hemophilia A. With these results, if a multispecific antigen-binding molecule having activity of functionally substituting for F.VIII is a molecule having a higher activity than hA69-KQ/hB26-PF/hAL-AQ, it may yield excellent blood coagulation-promoting activity, and excellent effects may be obtained as a pharmaceutical component for preventing and/or treating bleeding, a disease accompanying bleeding, or a disease caused by bleeding. To obtain excellent effects as the above-mentioned pharmaceutical component, for example, F.Xa generation-promoting activity measured under the conditions described in [Example 2] is preferably not less than that of hA69-KQ/hB26-PF/hAL-AQ, and in particular, the activity is more preferably the same as or not less than that of Q153-G4k/J142-G4h/L180-k. Herein, the “F.Xa generation-promoting activity” is the value obtained by subtracting the change in absorbance upon 20 minutes in a solvent from the change in absorbance upon 20 minutes in an antibody solution.

A preferred embodiment of the present invention is a multispecific antibody that functionally substitutes for F.VIII, which recognizes F.IX and/or F.IXa, and F.X.

The above-mentioned multispecific antibodies of the present invention are preferably antibodies which comprise H chain CDRs of anti-F.IX/F.IXa antibodies or CDRs functionally equivalent to them, and H chain CDRs of anti-F.X antibodies or CDRs functionally equivalent to them.

Furthermore, the antibodies of the present invention are preferably antibodies comprising an antigen-binding site having:
H chain CDR 1, 2, and 3 amino acid sequences of SEQ ID NOs 75, 76, and 77 (H chain CDRs of Q1), respectively;
H chain CDR 1, 2, and 3 amino acid sequences of SEQ ID NOs 78, 79, and 80 (H chain CDRs of Q31), respectively;
H chain CDR 1, 2, and 3 amino acid sequences of SEQ ID NOs 81, 82, and 83 (H chain CDRs of Q64), respectively;
H chain CDR 1, 2, and 3 amino acid sequences of SEQ ID NOs 84, 85, and 86 (H chain CDRs of Q85), respectively;
H chain CDR 1, 2, and 3 amino acid sequences of SEQ ID NOs 87, 88, and 89 (H chain CDRs of Q153), respectively;
H chain CDR 1, 2, and 3 amino acid sequences of SEQ ID NOs 90, 91, and 92 (H chain CDRs of Q354), respectively;
H chain CDR 1, 2, and 3 amino acid sequences of SEQ ID NOs 93, 94, and 95 (H chain CDRs of Q360), respectively;
H chain CDR 1, 2, and 3 amino acid sequences of SEQ ID NOs 96, 97, and 98 (H chain CDRs of Q405), respectively;
H chain CDR 1, 2, and 3 amino acid sequences of SEQ ID NOs 99, 100, and 101 (H chain CDRs of Q458), respectively;
H chain CDR 1, 2, and 3 amino acid sequences of SEQ ID NOs 102, 103, and 104 (H chain CDRs of Q460), respectively; or
H chain CDR 1, 2, and 3 amino acid sequences of SEQ ID NOs 105, 106, and 107 (H chain CDRs of Q499), respectively,
in an anti-F.IX/IXa antibody, or an antigen-binding site functionally equivalent to it, and an antigen-binding site comprising:
H chain CDR 1, 2, and 3 amino acid sequences of SEQ ID NOs 108, 109, and 110 (H chain CDRs of J232), respectively;
H chain CDR 1, 2, and 3 amino acid sequences of SEQ ID NOs 111, 112, and 113 (H chain CDRs of J259), respectively;
H chain CDR 1, 2, and 3 amino acid sequences of SEQ ID NOs 114, 115, and 116 (H chain CDRs of J268), respectively;
H chain CDR 1, 2, and 3 amino acid sequences of SEQ ID NOs 117, 118, and 119 (H chain CDRs of J300), respectively;
H chain CDR 1, 2, and 3 amino acid sequences of SEQ ID NOs 120, 121, and 122 (H chain CDRs of J321), respectively;
H chain CDR 1, 2, and 3 amino acid sequences of SEQ ID NOs 123, 124, and 125 (H chain CDRs of J326), respectively;
H chain CDR 1, 2, and 3 amino acid sequences of SEQ ID NOs 126, 127, and 128 (H chain CDRs of J327), respectively;
H chain CDR 1, 2, and 3 amino acid sequences of SEQ ID NOs 129, 130, and 131 (H chain CDRs of J339), respectively;
H chain CDR 1, 2, and 3 amino acid sequences of SEQ ID NOs 132, 133, and 134 (H chain CDRs of J334), respectively;
the amino acid sequences of H chain CDR 1, 2, and 3 amino acid sequences of SEQ ID NOs 135, 136, and 137 (H chain CDRs of J346), respectively; or
H chain CDR 1, 2, and 3 amino acid sequences of SEQ ID NOs 174, 175, and 176 (H chain CDRs of J142), respectively,
in an anti-F.X antibody, or an antigen-binding site functionally equivalent to it.

In the present invention, “antigen-binding sites are functionally equivalent” means that the activities of functionally substituting for F.VIII possessed by the multispecific antigen-binding molecules having the antigen-binding sites are equivalent.

In the present invention, the term “equivalent” does not necessarily have to mean the same degree of activity, and the activity may be enhanced, or the activity may be decreased as long as there is an activity higher than that of hA69-KQ/hB26-PF/hAL-AQ according to the measurement system described above, or preferably F.Xa generation-promoting activity measured under the conditions described in [Example 2] is equivalent to or not less than that of Q153-G4k/J142-G4h/L180-k.

The above-mentioned antibodies may have one or more amino acid substitutions, deletions, additions, and/or insertions in the variable region (CDR sequences and/or FR sequences) of the amino acid sequence as long as they have an activity higher than that of hA69-KQ/hB26-PF/hAL-AQ according to the measurement system described above at page 35, lines 11-30, or preferably F.Xa generation-promoting activity measured under the conditions described in [Example 2] is equivalent to or not less than that of Q153-G4k/J142-G4h/L180-k. A method of introducing mutations into proteins is well known to those skilled in the art as a method for introducing one or more amino acid substitutions, deletions, additions, and/or insertions into an amino acid sequence. For example, those skilled in the art can prepare a desired mutant functionally equivalent to a multispecific polypeptide multimer having the activity of functionally substituting for F.VIII by introducing appropriate mutations into the amino acid sequence using site-directed mutagenesis (Hashimoto-Gotoh, T, Mizuno, T, Ogasahara, Y, and Nakagawa, M. (1995) An oligodeoxyribonucleotide-directed dual amber method for site-directed mutagenesis. Gene 152: 271-275; Zoller, M J, and Smith, M. (1983) Oligonucleotide-directed mutagenesis of DNA fragments cloned into M13 vectors. Methods Enzymol. 100: 468-500; Kramer, W, Drutsa, V, Jansen, H W, Kramer, B, Pflugfelder, M, and Fritz, H J (1984) The gapped duplex DNA approach to oligonucleotide-directed mutation construction. Nucleic Acids Res. 12, 9441-9456; Kramer W, and Fritz H J (1987) Oligonucleotide-directed construction of mutations via gapped duplex DNA Methods. Enzymol. 154: 350-367; and Kunkel, TA (1985) Rapid and efficient site-specific mutagenesis without phenotypic selection. Proc Natl Acad Sci USA. 82: 488-492) and such.

As such, antibodies of the present invention also include antibodies with one or more amino acid mutations in the variable region, and having an activity higher than that of hA69-KQ/hB26-PF/hAL-AQ according to the measurement system described above at page 35, lines 11-30, or preferably F.Xa generation-promoting activity measured under the conditions described in [Example 2] is equivalent to or not less than that of Q153-G4k/J142-G4h/L180-k.

When an amino acid residue is altered, the amino acid is preferably mutated for a different amino acid(s) that conserves the properties of the amino acid side-chain. Examples of amino acid side chain properties are: hydrophobic amino acids (A, I, L, M, F, P, W, Y, and V), hydrophilic amino acids (R, D, N, C, E, Q, G, H, K, S, and T), amino acids containing aliphatic side chains (G, A, V, L, I, and P), amino acids containing hydroxyl group-containing side chains (S, T, and Y), amino acids containing sulfur-containing side chains (C and M), amino acids containing carboxylic acid- and amide-containing side chains (D, N, E, and Q), amino acids containing basic side chains (R, K, and H), and amino acids containing aromatic side chains (H, F, Y, and W) (amino acids are represented by one-letter codes in parentheses). Amino acid substitutions within each group are called conservative substitutions. It is already known that a polypeptide containing a modified amino acid sequence in which one or more amino acid residues in a given amino acid sequence are deleted, added, and/or substituted with other amino acids can retain the original biological activity (Mark, D. F. et al., Proc. Natl. Acad. Sci. USA; (1984) 81: 5662-6; Zoller, M. J. and Smith, M., Nucleic Acids Res. (1982) 10: 6487-500; Wang, A. et al., Science (1984) 224: 1431-3; Dalbadie-McFarland, G. et al., Proc. Natl. Acad. Sci. USA (1982) 79: 6409-13). Such mutants have an amino acid identity of at least 70%, more preferably at least 75%, even more preferably at least 80%, still more preferably at least 85%, yet more preferably at least 90%, and most preferably at least 95%, with the variable regions (for example, CDR sequences, FR sequences, or whole variable regions) of the present invention. Herein, sequence identity is defined as the percentage of residues identical to those in the original amino acid sequence of the heavy chain variable region or light chain variable region, determined after the sequences are aligned and gaps are appropriately introduced to maximize the sequence identity as necessary. The identity of amino acid sequences can be determined by the method described below.

Alternatively, the amino acid sequences of variable regions that have a substitution, deletion, addition, and/or insertion of one or more amino acids in the amino acid sequence of the variable regions (CDR sequences and/or FR sequences) and have an activity higher than that of hA69-KQ/hB26-PF/hAL-AQ according to the measurement system described above at page 35, lines 11-30, or preferably F.Xa generation-promoting activity measured under the conditions described in [Example 2] is equivalent to or not less than that of Q153-G4k/J142-G4h/L180-k can be obtained from nucleic acids that hybridize under stringent conditions to nucleic acid composed of the nucleotide sequence encoding the amino acid sequence of the variable regions. Stringent hybridization conditions to isolate a nucleic acid that hybridizes under stringent conditions to a nucleic acid that includes the nucleotide sequence encoding the amino acid sequence of the variable regions include, for example, the conditions of 6 M urea, 0.4% SDS, 0.5×SSC, and 37° C., or hybridization conditions with stringencies equivalent thereto. With more stringent conditions, for example, the conditions of 6 M urea, 0.4% SDS, 0.1×SSC, and 42° C., isolation of nucleic acids with a much higher homology can be expected. The sequences of the isolated nucleic acids can be determined by the known methods described below. The overall nucleotide sequence homology of the isolated nucleic acid is at least 50% or higher sequence identity, preferably 70% or higher, more preferably 90% or higher (for example, 95%, 96%, 97%, 98%, 99%, or higher).

Nucleic acids that hybridize under stringent conditions to a nucleic acid composed of the nucleotide sequence encoding the amino acid sequence of the variable regions can also be isolated using, instead of the above-described methods using hybridization techniques, gene amplification methods such as polymerase chain reaction (PCR) using primers synthesized based on the information of nucleotide sequence encoding the amino acid sequence of the variable regions.

The identity of one nucleotide sequence or amino acid sequence to another can be determined using the algorithm BLAST, by Karlin and Altschul (Proc. Natl. Acad. Sci. USA (1993) 90: 5873-7). Programs such as BLASTN and BLASTX were developed based on this algorithm (Altschul et al., J. Mol. Biol. (1990) 215: 403-10). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); http://www.ncbi.nlm.nih.gov).

The present invention also provides antibodies that bind to an epitope overlapping with an epitope bound by the antibodies described above.

Whether an antibody recognizes an epitope overlapping with an epitope that is recognized by another antibody can be confirmed by the competition between the two antibodies against the epitope. Competition between the antibodies can be evaluated by competitive binding assays using means such as enzyme-linked immunosorbent assay (ELISA), fluorescence energy transfer method (FRET), and fluorometric microvolume assay technology (FMAT (Registered trademark)). The amount of antibodies bound to an antigen indirectly correlate with the binding ability of candidate competitor antibodies (test antibodies) that competitively bind to the overlapping epitope. In other words, as the amount of or the affinity of test antibodies against the overlapping epitope increases, the amount of antibodies bound to the antigen decreases, and the amount of test antibodies bound to the antigen increases. Specifically, appropriately labeled antibodies and antibodies to be evaluated are simultaneously added to the antigens, and the thus bound antibodies are detected using the label. The amount of antibodies bound to the antigen can be easily determined by labeling the antibodies beforehand. This label is not particularly limited, and the labeling method is selected according to the assay technique used. The labeling method includes fluorescent labeling, radiolabeling, enzymatic labeling, and such.

For example, fluorescently labeled antibodies and unlabeled antibodies or test antibodies are simultaneously added to beads immobilized with F.IX, F.IXa or F.X, and the labeled antibodies are detected by fluorometric microvolume assay technology.

Herein, the “antibody that binds to the overlapping epitope” refers to an antibody that can reduce the binding of the labeled antibody by at least 50% at a concentration that is usually 100 times higher, preferably 80 times higher, more preferably 50 times higher, even more preferably 30 times higher, and still more preferably 10 times higher than a concentration at which the non-labeled antibody reduces the binding of the labeled antibody by 50% (IC₅₀).

Multispecific antigen-binding molecules, which have antigen-binding sites of antibodies that bind to epitopes overlapping with epitopes bound by the above-mentioned antibodies, may yield an excellent activity of functionally substituting for F.VIII. Furthermore, in antigen-binding sites of antibodies that bind to epitopes overlapping with epitopes bound by the above-mentioned antibodies, one or more amino acids may be altered to obtain a better activity of functionally substituting for F.VIII. Multispecific antigen-binding molecules having a better activity of functionally substituting for F.VIII can be obtained by altering the amino acids of the antigen-binding sites and selecting multispecific antigen-binding molecules having an activity higher than that of hA69-KQ/hB26-PF/hAL-AQ according to the measurement system described above, or preferably having an F.Xa generation-promoting activity measured under the conditions described in [Example 2] that is equivalent to or not less than that of Q153-G4k/J142-G4h/L180-k. To obtain an excellent activity of functionally substituting for F.VIII of the present invention, the following amino acid alterations are particularly preferred.
(1) At least one amino acid residue selected from the amino acid residues at positions 34, 35, 49, 61, 62, 96, 98, 100, 100b, and 102 by Kabat numbering in the H chain of the antibody that recognizes F.IX and/or F.IXa is substituted with a different amino acid.
(2) At least one amino acid residue selected from the amino acid residues at positions 35, 53, 73, 76, 96, 98, 100, and 100a by Kabat numbering in the H chain of the antibody that recognizes F.X is substituted with a different amino acid.
(3) At least one amino acid residue selected from the amino acid residues at positions 27, 30, 31, 32, 50, 52, 53, 54, 55, 92, 93, 94, and 95 by Kabat numbering in the antibody L chain is substituted with a different amino acid.

Furthermore, in the present invention, preferred antibody amino acids for obtaining a better activity of functionally substituting for F.VIII include those mentioned in (4) to (6) below.

Regarding these amino acids, the antibody H chain may originally have such amino acids, or antibody H chain amino acids may be modified to have such a sequence.
(4) An antibody H chain which recognizes F.IX and/or F.IXa, wherein, by Kabat numbering, the amino acid residue at position 34 is isoleucine, the amino acid residue at position 35 is asparagine, glutamine, or serine, the amino acid residue at position 49 is serine, the amino acid residue at position 61 is arginine, the amino acid residue at position 62 is glutamic acid, the amino acid residue at position 96 is serine or threonine, the amino acid residue at position 98 is lysine or arginine, the amino acid residue at position 100 is phenylalanine or tyrosine, the amino acid residue at position 100b is glycine, or the amino acid residue at position 102 is tyrosine.
(5) An antibody H chain which recognizes F.X, wherein, by Kabat numbering, the amino acid residue at position 35 is aspartic acid, the amino acid residue at position 53 is arginine, the amino acid residue at position 73 is lysine, the amino acid residue at position 76 is glycine, the amino acid residue at position 96 is lysine or arginine, the amino acid residue at position 98 is tyrosine, the amino acid residue at position 100 is tyrosine, or the amino acid residue at position 100a is histidine.
(6) An antibody L chain, wherein, by Kabat numbering, the amino acid residue at position 27 is lysine or arginine, the amino acid residue at position 30 is glutamic acid, the amino acid residue at position 31 is arginine, the amino acid residue at position 32 is glutamine, the amino acid residue at position 50 is arginine or glutamine, the amino acid residue at position 52 is serine, the amino acid residue at position 53 is arginine, the amino acid residue at position 54 is lysine, the amino acid residue at position 55 is glutamic acid, the amino acid residue at position 92 is serine, the amino acid residue at position 93 is serine, the amino acid residue at position 94 is proline, or the amino acid residue at position 95 is proline.

Among the above-mentioned antibody amino acid residues of (1) to (6), favorable positions of amino acid residues for obtaining a particularly excellent F.VIII-like activity are shown in the following (1) to (3).
(1) Amino acid residues at positions 34, 35, 61, 98, 100, and 100b, particularly amino acid residues at positions 61 and 100, by Kabat numbering in the H chain of the antibody that recognizes F.IX and/or F.IXa.
(2) Amino acid residues at positions 35, 53, 73, 96, 98, 100, and 100a by Kabat numbering in the H chain of the antibody that recognizes F.X.
(3) Amino acid residues at positions 27, 30, 31, 32, 50, 52, 53, 93, 94, and 95, and particularly amino acid residues at positions 27, 30, 31, 50, 53, 94, and 95, by Kabat numbering in the antibody L chain.

Specifically, the present invention provides multispecific antigen-binding molecules, wherein a first polypeptide comprises any of the antibody H chains selected from the following (a1) to (a14) and any of the antibody L chains selected from the following (c1) to (c10), and the second polypeptide comprises any of the antibody H chains selected from the following (b1) to (b12) and any of the antibody L chains selected from the following (c1) to (c10):
(a1) an antibody H chain consisting of the amino acid sequence of SEQ ID NO: 1 (Q1-G4k);
(a2) an antibody H chain consisting of the amino acid sequence of SEQ ID NO: 2 (Q31-z7);
(a3) an antibody H chain consisting of the amino acid sequence of SEQ ID NO: 3 (Q64-z55);
(a4) an antibody H chain consisting of the amino acid sequence of SEQ ID NO: 10 (Q64-z7);
(a5) an antibody H chain consisting of the amino acid sequence of SEQ ID NO: 11 (Q85-G4k);
(a6) an antibody H chain consisting of the amino acid sequence of SEQ ID NO: 12 (Q153-G4k);
(a7) an antibody H chain consisting of the amino acid sequence of SEQ ID NO: 13 (Q354-z106);
(a8) an antibody H chain consisting of the amino acid sequence of SEQ ID NO: 14 (Q360-G4k);
(a9) an antibody H chain consisting of the amino acid sequence of SEQ ID NO: 15 (Q360-z118);
(a10) an antibody H chain consisting of the amino acid sequence of SEQ ID NO: 16 (Q405-G4k);
(a11) an antibody H chain consisting of the amino acid sequence of SEQ ID NO: 17 (Q458-z106);
(a12) an antibody H chain consisting of the amino acid sequence of SEQ ID NO: 18 (Q460-z121);
(a13) an antibody H chain consisting of the amino acid sequence of SEQ ID NO: 19 (Q499-z118);
(a14) an antibody H chain consisting of the amino acid sequence of SEQ ID NO: 20 (Q499-z121);
(b1) an antibody H chain consisting of the amino acid sequence of SEQ ID NO: 4 (J268-G4h);
(b2) an antibody H chain consisting of the amino acid sequence of SEQ ID NO: 5 (J321-G4h);
(b3) an antibody H chain consisting of the amino acid sequence of SEQ ID NO: 6 (J326-z107);
(b4) an antibody H chain consisting of the amino acid sequence of SEQ ID NO: 7 (J344-z107);
(b5) an antibody H chain consisting of the amino acid sequence of SEQ ID NO: 21 (J232-G4h);
(b6) an antibody H chain consisting of the amino acid sequence of SEQ ID NO: 22 (J259-z107);
(b7) an antibody H chain consisting of the amino acid sequence of SEQ ID NO: 23 (J300-z107);
(b8) an antibody H chain consisting of the amino acid sequence of SEQ ID NO: 24 (J327-z107);
(b9) an antibody H chain consisting of the amino acid sequence of SEQ ID NO: 25 (J327-z119);
(b10) an antibody H chain consisting of the amino acid sequence of SEQ ID NO: 26 (J339-z119);
(b11) an antibody H chain consisting of the amino acid sequence of SEQ ID NO: 27 (J346-z107);
(b12) an antibody H chain consisting of the amino acid sequence of SEQ ID NO: 170 (J142-G4h);
(c1) an antibody L chain consisting of the amino acid sequence of SEQ ID NO: 8 (L2-k);
(c2) an antibody L chain consisting of the amino acid sequence of SEQ ID NO: 9 (L45-k);
(c3) an antibody L chain consisting of the amino acid sequence of SEQ ID NO: 28 (L248-k);
(c4) an antibody L chain consisting of the amino acid sequence of SEQ ID NO: 29 (L324-k);
(c5) an antibody L chain consisting of the amino acid sequence of SEQ ID NO: 30 (L334-k);
(c6) an antibody L chain consisting of the amino acid sequence of SEQ ID NO: 31 (L377-k);
(c7) an antibody L chain consisting of the amino acid sequence of SEQ ID NO: 32 (L404-k);
(c8) an antibody L chain consisting of the amino acid sequence of SEQ ID NO: 33 (L406-k);
(c9) an antibody L chain consisting of the amino acid sequence of SEQ ID NO: 34 (L408-k); and
(c10) an antibody L chain consisting of the amino acid sequence of SEQ ID NO: 171 (L180-k).

The present invention also provides multispecific antigen-binding molecules, wherein the first polypeptide comprises an antigen-binding site which binds to an epitope overlapping with an epitope that binds to an antibody consisting of the antibody H chain of any one of (a1) to (a14) and the antibody L chain of any one of (c1) to (c10) described above, and the second polypeptide comprises an antigen-binding site which binds to an epitope overlapping with an epitope that binds to an antibody consisting of the antibody H chain of any one of (b1) to (b12) and the antibody L chain of any one of (c1) to (c10) described above.

Furthermore, the present invention provides multispecific antigen-binding molecules, wherein the first polypeptide comprises any one antibody H chain selected from the following (e1) to (e3), the second polypeptide comprises any one antibody H chain selected from the following (f1) to (f3), and the third polypeptide and the fourth polypeptide comprise any one antibody L chain selected from the following (g1) to (g4):
(e1) an H chain of an antibody which binds to an epitope overlapping with an epitope bound by an antibody consisting of an antibody H chain of any one of (a1) to (a14) and an antibody L chain of any one of (c1) to (c10) described above;
(e2) an antibody H chain, wherein at least one amino acid residue selected from the amino acid residues at positions 34, 35, 49, 61, 62, 96, 98, 100, 100b, and 102 by Kabat numbering in any one antibody H chain selected from (e1) described above is substituted with another amino acid;
(e3) an antibody H chain, wherein by Kabat numbering, the amino acid residue at position 34 is isoleucine, the amino acid residue at position 35 is asparagine, glutamine, or serine, the amino acid residue at position 49 is serine, the amino acid residue at position 61 is arginine, the amino acid residue at position 62 is glutamic acid, the amino acid residue at position 96 is serine or threonine, the amino acid residue at position 98 is lysine or arginine, the amino acid residue at position 100 is phenylalanine or tyrosine, the amino acid residue at position 100b is glycine, or the amino acid residue at position 102 is tyrosine in any antibody H chain selected from (e1) described above;
(f1) an H chain of an antibody which binds to an epitope overlapping with an epitope bound by an antibody consisting of an antibody H chain of any of (b1) to (b12) described above and an antibody L chain of any of (c1) to (c10) described above;
(f2) an antibody H chain, wherein at least one amino acid residue selected from the amino acid residues at positions 35, 53, 73, 76, 96, 98, 100, and 100a by Kabat numbering in any antibody H chain of (f1) described above is substituted with another amino acid;
(f3) an antibody H chain, wherein by Kabat numbering, the amino acid residue at position 35 is aspartic acid, the amino acid residue at position 53 is arginine, the amino acid residue at position 73 is lysine, the amino acid residue at position 76 is glycine, the amino acid residue at position 96 is lysine or arginine, the amino acid residue at position 98 is tyrosine, the amino acid residue at position 100 is tyrosine, or the amino acid residue at position 100a is histidine in any one antibody H chain selected from (f1) described above;
(g1) an L chain of an antibody which binds to an epitope overlapping with an epitope bound by an antibody which consists of an antibody H chain of any one of (a1) to (a14) and an antibody L chain of any one of (c1) to (c10) described above;
(g2) an L chain of an antibody which binds to an epitope overlapping with an epitope bound by an antibody which consists of an antibody H chain of any one of (b1) to (b12) and an antibody L chain of any one of (c1) to (c10) described above;
(g3) an antibody L chain, wherein at least one amino acid residue selected from the amino acid residues at positions 27, 30, 31, 32, 50, 52, 53, 54, 55, 92, 93, 94, and 95 by Kabat numbering in the antibody L chain of either (g1) or (g2) described above is substituted with another amino acid; and
(g4) an antibody L chain, wherein by Kabat numbering, the amino acid residue at position 27 is lysine or arginine, the amino acid residue at position 30 is glutamic acid, the amino acid residue at position 31 is arginine, the amino acid residue at position 32 is glutamine, the amino acid residue at position 50 is arginine or glutamine, the amino acid residue at position 52 is serine, the amino acid residue at position 53 is arginine, the amino acid residue at position 54 is lysine, the amino acid residue at position 55 is glutamic acid, the amino acid residue at position 92 is serine, the amino acid residue at position 93 is serine, the amino acid residue at position 94 is proline, or the amino acid residue at position 95 is proline in the antibody L chain of either (g1) or (g2) described above.

Amino acid substitutions can be performed on the antibodies (clones) of the present invention to avoid deamidation, methionine oxidation, and such, or to structurally stabilize the antibodies.

The method for obtaining multispecific antigen-binding molecules of the present invention is not particularly limited, and may be any method. Bispecific antibodies can be generated according to the methods described in WO 2006/109592, WO 2005/035756, WO 2006/106905, or WO 2007/114325, which are known as examples of the method for producing the bispecific antibodies; and then desired antibodies having a cofactor function-substituting activity can be selected and obtained.

For example, the bispecific antibody described in any of the following (a) to (u) is provided by the present invention:
(a) a bispecific antibody (Q1-G4k/J268-G4h/L45-k), wherein the first polypeptide is an H chain consisting of the amino acid sequence of SEQ ID NO: 1, the second polypeptide is an H chain consisting of the amino acid sequence of SEQ ID NO: 4, and the third polypeptide and the fourth polypeptide are a commonly shared L chain of SEQ ID NO: 9;
(b) a bispecific antibody (Q1-G4k/J321-G4h/L45-k), wherein the first polypeptide is an H chain consisting of the amino acid sequence of SEQ ID NO: 1, the second polypeptide is an H chain consisting of the amino acid sequence of SEQ ID NO: 5, and the third polypeptide and the fourth polypeptide are a commonly shared L chain of SEQ ID NO: 9;
(c) a bispecific antibody (Q31-z7/J326-z107/L2-k), wherein the first polypeptide is an H chain consisting of the amino acid sequence of SEQ ID NO: 2, the second polypeptide is an H chain consisting of the amino acid sequence of SEQ ID NO: 6, and the third polypeptide and the fourth polypeptide are a commonly shared L chain of SEQ ID NO: 8;
(d) a bispecific antibody (Q64-z55/J344-z107/L45-k), wherein the first polypeptide is an H chain consisting of the amino acid sequence of SEQ ID NO: 3, the second polypeptide is an H chain consisting of the amino acid sequence of SEQ ID NO: 7, and the third polypeptide and the fourth polypeptide are a commonly shared L chain of SEQ ID NO: 9;
(e) a bispecific antibody (Q64-z7/J326-z107/L334-k), wherein the first polypeptide is an H chain consisting of the amino acid sequence of SEQ ID NO: 10, the second polypeptide is an H chain consisting of the amino acid sequence of SEQ ID NO: 6, and the third polypeptide and the fourth polypeptide are a commonly shared L chain of SEQ ID NO: 30;
(f) a bispecific antibody (Q64-z7/J344-z107/L406-k), wherein the first polypeptide is an H chain consisting of the amino acid sequence of SEQ ID NO: 10, the second polypeptide is an H chain consisting of the amino acid sequence of SEQ ID NO: 7, and the third polypeptide and the fourth polypeptide are a commonly shared L chain of SEQ ID NO: 33;
(g) a bispecific antibody (Q85-G4k/J268-G4h/L406-k), wherein the first polypeptide is an H chain consisting of the amino acid sequence of SEQ ID NO: 11, the second polypeptide is an H chain consisting of the amino acid sequence of SEQ ID NO: 4, and the third polypeptide and the fourth polypeptide are a commonly shared L chain of SEQ ID NO: 33;
(h) a bispecific antibody (Q85-G4k/J321-G4h/L334-k), wherein the first polypeptide is an H chain consisting of the amino acid sequence of SEQ ID NO: 11, the second polypeptide is an H chain consisting of the amino acid sequence of SEQ ID NO: 5, and the third polypeptide and the fourth polypeptide are a commonly shared L chain of SEQ ID NO: 30;
(i) a bispecific antibody (Q153-G4k/J232-G4h/L406-k), wherein the first polypeptide is an H chain consisting of the amino acid sequence of SEQ ID NO: 12, the second polypeptide is an H chain consisting of the amino acid sequence of SEQ ID NO: 21, and the third polypeptide and the fourth polypeptide are a commonly shared L chain of SEQ ID NO: 33;
(j) a bispecific antibody (Q354-z106/J259-z107/L324-k), wherein the first polypeptide is an H chain consisting of the amino acid sequence of SEQ ID NO: 13, the second polypeptide is an H chain consisting of the amino acid sequence of SEQ ID NO: 22, and the third polypeptide and the fourth polypeptide are a commonly shared L chain of SEQ ID NO: 29;
(k) a bispecific antibody (Q360-G4k/J232-G4h/L406-k), wherein the first polypeptide is an H chain consisting of the amino acid sequence of SEQ ID NO: 14, the second polypeptide is an H chain consisting of the amino acid sequence of SEQ ID NO: 21, and the third polypeptide and the fourth polypeptide are a commonly shared L chain of SEQ ID NO: 33;
(l) a bispecific antibody (Q360-z118/J300-z107/L334-k), wherein the first polypeptide is an H chain consisting of the amino acid sequence of SEQ ID NO: 15, the second polypeptide is an H chain consisting of the amino acid sequence of SEQ ID NO: 23, and the third polypeptide and the fourth polypeptide are a commonly shared L chain of SEQ ID NO: 30;
(m) a bispecific antibody (Q405-G4k/J232-G4h/L248-k), wherein the first polypeptide is an H chain consisting of the amino acid sequence of SEQ ID NO: 16, the second polypeptide is an H chain consisting of the amino acid sequence of SEQ ID NO: 21, and the third polypeptide and the fourth polypeptide are a commonly shared L chain of SEQ ID NO: 28;
(n) a bispecific antibody (Q458-z106/J346-z107/L408-k), wherein the first polypeptide is an H chain consisting of the amino acid sequence of SEQ ID NO: 17, the second polypeptide is an H chain consisting of the amino acid sequence of SEQ ID NO: 27, and the third polypeptide and the fourth polypeptide are a commonly shared L chain of SEQ ID NO: 34;
(o) a bispecific antibody (Q460-z121/J327-z119/L334-k), wherein the first polypeptide is an H chain consisting of the amino acid sequence of SEQ ID NO: 18, the second polypeptide is an H chain consisting of the amino acid sequence of SEQ ID NO: 25, and the third polypeptide and the fourth polypeptide are a commonly shared L chain of SEQ ID NO: 30;
(p) a bispecific antibody (Q499-z118/J327-z107/L334-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 30;
(q) a bispecific antibody (Q499-z118/J327-z107/L377-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 31;
(r) a bispecific antibody (Q499-z118/J346-z107/L248-k), wherein the first polypeptide is an H chain consisting of the amino acid sequence of SEQ ID NO: 19, the second polypeptide is an H chain consisting of the amino acid sequence of SEQ ID NO: 27, and the third polypeptide and the fourth polypeptide are a commonly shared L chain of SEQ ID NO: 28;
(s) a bispecific antibody (Q499-z121/J327-z119/L404-k), wherein the first polypeptide is an H chain consisting of the amino acid sequence of SEQ ID NO: 20, the second polypeptide is an H chain consisting of the amino acid sequence of SEQ ID NO: 25, and the third polypeptide and the fourth polypeptide are a commonly shared L chain of SEQ ID NO: 32;
(t) a bispecific antibody (Q499-z121/J339-z119/L377-k), wherein the first polypeptide is an H chain consisting of the amino acid sequence of SEQ ID NO: 20, the second polypeptide is an H chain consisting of the amino acid sequence of SEQ ID NO: 26, and the third polypeptide and the fourth polypeptide are a commonly shared L chain of SEQ ID NO: 31; and
(u) a bispecific antibody (Q153-G4k/J142-G4h/L180-k), wherein the first polypeptide is an H chain consisting of the amino acid sequence of SEQ ID NO: 12, the second polypeptide is an H chain consisting of the amino acid sequence of SEQ ID NO: 170, and the third polypeptide and the fourth polypeptide are a commonly shared L chain of SEQ ID NO: 171.

Amino acid sequences, molecular weights, isoelectric points, or presence or absence and form of sugar chains of the antibodies of the present invention vary depending on cells or hosts that produce the antibodies or purification methods described later. However, as long as the obtained antibodies have functions equivalent to the antibodies of the present invention, they are included in the present invention. For example, when an antibody of the present invention is expressed in prokaryotic cells such as E. coli, a methionine residue will be added to the N terminus of the original antibody amino acid sequence. Antibodies of the present invention also comprise such antibodies.

Bispecific antibodies of the present invention can be produced by methods known to those skilled in the art.

Based on the obtained sequence of the anti-F.IX/F.IXa antibody or anti-F.X antibody, the anti-F.IX/F.IXa antibody or anti-F.X antibody can be prepared, for example, by genetic recombination techniques known to those skilled in the art. Specifically, a polynucleotide encoding an antibody can be constructed based on the sequence of the anti-F.IX/F.IXa antibody or anti-F.X antibody, inserted into an expression vector, and then expressed in appropriate host cells (see for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; Bird, R. E. and Walker, B. W., Trends Biotechnol. (1991) 9, 132-137).

The vectors include M13 vectors, pUC vectors, pBR322, pBluescript, and pCR-Script. Alternatively, when aiming to subclone and excise cDNA, the vectors include, for example, pGEM-T, pDIRECT, and pT7, in addition to the vectors described above. Expression vectors are particularly useful when using vectors for producing the antibodies of the present invention. For example, when aiming for expression in E. coli such as JM109, DH5α, HB101, and XL1-Blue, the expression vectors not only have the characteristics that allow vector amplification in E. coli, but must also carry a promoter that allows efficient expression in E. coli, for example, lacZ promoter (Ward et al., Nature (1989) 341: 544-546; FASEB J. (1992) 6: 2422-2427), araB promoter (Better et al., Science (1988) 240: 1041-1043), T7 promoter or such. Such vectors include pGEX-5X-1 (Pharmacia), “QIAexpress system” (Qiagen), pEGFP, or pET (in this case, the host is preferably BL21 that expresses T7 RNA polymerase) in addition to the vectors described above.

The expression plasmid vectors may contain signal sequences for antibody secretion. As a signal sequence for antibody secretion, a pelB signal sequence (Lei, S. P. et al J. Bacteriol. (1987) 169: 4379) may be used when a protein is secreted into the E. coli periplasm. The vector can be introduced into host cells by calcium chloride or electroporation methods, for example.

In addition to vectors for E. coli, the vectors for producing the antibodies of the present invention include mammalian expression vectors (for example, pcDNA3 (Invitrogen), pEF-BOS (Nucleic Acids. Res. 1990, 18(17): p5322), pEF, and pCDM8), insect cell-derived expression vectors (for example, the “Bac-to-BAC baculovirus expression system” (Gibco-BRL) and pBacPAK8), plant-derived expression vectors (for example, pMH1 and pMH2), animal virus-derived expression vectors (for example, pHSV, pMV, and pAdexLcw), retroviral expression vectors (for example, pZIPneo), yeast expression vectors (for example, “Pichia Expression Kit” (Invitrogen), pNV 11, and SP-Q01), and Bacillus subtilis expression vectors (for example, pPL608 and pKTH50), for example.

When aiming for expression in animal cells such as CHO, COS, and NIH3T3 cells, the expression plasmid vectors must have a promoter essential for expression in cells, for example, SV40 promoter (Mulligan et al., Nature (1979) 277: 108), MMLV-LTR promoter, EF1α promoter (Mizushima et al., Nucleic Acids Res. (1990) 18: 5322), and CMV promoter, and more preferably they have a gene for selecting transformed cells (for example, a drug resistance gene that allows evaluation using an agent (neomycin, G418, or such)). Vectors with such characteristics include pMAM, pDR2, pBK-RSV, pBK-CMV, pOPRSV, and pOP13, for example.

In addition, the following method can be used for stable gene expression and gene amplification in cells: CHO cells deficient in a nucleic acid synthesis pathway are introduced with a vector that carries a DHFR gene which compensates for the deficiency (for example, pSV2-dhfr (Molecular Cloning 2^{nd} edition, Cold Spring Harbor Laboratory Press, 1989)), and the vector is amplified using methotrexate (MTX). Alternatively, the following method can be used for transient gene expression: COS cells with a gene expressing SV40 T antigen on their chromosome are transformed with a vector with an SV40 replication origin (pcD and such). Replication origins derived from polyoma virus, adenovirus, bovine papilloma virus (BPV), and such can also be used. To amplify gene copy number in host cells, the expression vectors may further carry selection markers such as aminoglycoside transferase (APH) gene, thymidine kinase (TK) gene, E. coli xanthine-guanine phosphoribosyltransferase (Ecogpt) gene, and dihydrofolate reductase (dhfr) gene.

The antibodies of the present invention obtained by the methods described above can be isolated from inside host cells or from outside the cells (the medium, or such), and purified to homogeneity. The antibodies can be isolated and purified by methods routinely used for isolating and purifying antibodies, and the type of method is not limited. For example, the antibodies can be isolated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectrofocusing, dialysis, recrystallization, and such.

The chromatographies include, for example, affinity chromatography, ion exchange chromatography, hydrophobic chromatography, gel filtration, reverse phase chromatography, and adsorption chromatography (Strategies for Protein Purification and Characterization: A Laboratory Course Manual. Ed Daniel R. Marshak et al., Cold Spring Harbor Laboratory Press, 1996). The chromatographic methods described above can be conducted using liquid chromatography, for example, HPLC and FPLC. Columns that can be used for affinity chromatography include protein A columns and protein G columns. Columns using protein A include, for example, Hyper D, POROS, and Sepharose FF (GE Amersham Biosciences). The present invention includes antibodies that are highly purified using these purification methods.

The obtained antibodies can be purified to homogeneity. Separation and purification of the antibodies can be performed using conventional separation and purification methods used for ordinary proteins. For example, the antibodies can be separated and purified by appropriately selecting and combining column chromatography such as affinity chromatography, filtration, ultrafiltration, salting-out, dialysis, SDS polyacrylamide gel electrophoresis, isoelectric focusing, and such, without limitation (Antibodies: A Laboratory Manual. Ed Harlow and David Lane, Cold Spring Harbor Laboratory, 1988). Columns used for affinity chromatography include, for example, protein A columns and protein G columns.

In one embodiment of antibodies of the present invention, since the antibodies of the present invention functionally substitute for cofactor F.VIII, they are expected to become effective pharmaceutical agents against diseases resulting from decrease in activity (function) of this cofactor. Examples of the above-mentioned diseases include bleeding, diseases accompanying bleeding, or a disease caused by bleeding. In particular, there may have excellent therapeutic effects on hemophilias, in which bleeding disorders are caused by a deficiency or decrease of F.VIII/F.VIIIa function. Among the hemophilias, they are expected to become excellent therapeutic agents for hemophilia A, in which bleeding disorders are caused by a hereditary deficiency or decrease of F.VIII/F.VIIIa function.

The present invention provides (pharmaceutical) compositions comprising the antibodies of the present invention and pharmaceutically acceptable carriers. For example, the antibodies of the present invention that recognize both F.IX or F.IXa and F.X, and functionally substitute for F.VIII are expected to become pharmaceuticals (pharmaceutical compositions) or pharmaceutical agents for preventing and/or treating bleeding, diseases accompanying bleeding, diseases caused by bleeding, and the like.

In the context of the present invention, bleeding, diseases accompanying bleeding, and/or diseases caused by bleeding preferably refer to diseases that develop and/or progress due to reduction or deficiency in activity of F.VIII and/or activated coagulation factor VIII (F.VIIIa). Such diseases include the above-described hemophilia A, diseases in which an inhibitor against F.VIII/F.VIIIa appear, acquired hemophilia, von Willebrand's disease, and such, but are not particularly limited thereto.

Pharmaceutical compositions used for therapeutic or preventive purposes, which comprise antibodies of the present invention as active ingredients, can be formulated by mixing, if necessary, with suitable pharmaceutically acceptable carriers, vehicles, and such that are inactive against the antibodies. For example, sterilized water, physiological saline, stabilizers, excipients, antioxidants (such as ascorbic acid), buffers (such as phosphate, citrate, histidine, and other organic acids), antiseptics, surfactants (such as PEG and Tween), chelating agents (such as EDTA), and binders may be used. They may also comprise other low-molecular-weight polypeptides, proteins such as serum albumin, gelatin, and immunoglobulins, amino acids such as glycine, glutamine, asparagine, glutamic acid, asparagic acid, methionine, arginine, and lysine, sugars and carbohydrates such as polysaccharides and monosaccharides, and sugar alcohols such as mannitol and sorbitol. When preparing an aqueous solution for injection, physiological saline and isotonic solutions comprising glucose and other adjuvants such as D-sorbitol, D-mannose, D-mannitol, and sodium chloride may be used, and if necessary, in combination with appropriate solubilizers such as alcohol (for example, ethanol), polyalcohols (such as propylene glycol and PEG), and nonionic surfactants (such as polysorbate 80, polysorbate 20, poloxamer 188, and HCO-50). By mixing hyaluronidase into the formulation, a larger fluid volume can be administered subcutaneously (Expert Opin Drug Deliv. 2007 July; 4(4): 427-40).

If necessary, antibodies of the present invention may be encapsulated in microcapsules (e.g., those made of hydroxymethylcellulose, gelatin, and poly(methylmetacrylate)), or incorporated as components of colloidal drug delivery systems (e.g., liposomes, albumin microspheres, microemulsion, nanoparticles, and nanocapsules) (see, for example, “Remington's Pharmaceutical Science 16th edition”, Oslo Ed. (1980)). Methods for preparing the pharmaceutical agents as controlled-release pharmaceutical agents are also well known, and such methods may be applied to the antibodies of the present invention (Langer et al., J. Biomed. Mater. Res. 15: 267-277 (1981); Langer, Chemtech. 12: 98-105 (1982); U.S. Pat. No. 3,773,919; European Patent Application Publication No. EP 58,481; Sidman et al., Biopolymers 22: 547-556 (1983); EP 133,988).

The dose of a pharmaceutical composition of the present invention may be appropriately determined by considering the dosage form, method of administration, patient age and body weight, symptoms of the patient, type of the disease, and degree of progress of the disease, and is ultimately decided by physicians. Generally, the daily dose for an adult is 0.1 mg to 2,000 mg at once or in several portions. The dose is more preferably 0.2 to 1,000 mg/day, even more preferably 0.5 to 500 mg/day, still more preferably 1 to 300 mg/day, yet more preferably 3 to 100 mg/day, and most preferably 5 to 50 mg/day. These doses may vary, depending on the patient body weight and age, and the method of administration; however, selection of suitable dosage is well within the purview of those skilled in the art. Similarly, the dosing period may be appropriately determined depending on the therapeutic progress.

Furthermore, the present invention provides genes or nucleic acids encoding the antibodies of the present invention. In addition, gene therapy may be performed by incorporating genes or nucleic acids encoding the antibodies of the present invention into vectors for gene therapy. In addition to direct administration using naked plasmids, methods of administration include administration after packaging into liposomes and such, forming a variety of virus vectors such as retrovirus vectors, adenovirus vectors, vaccinia virus vectors, poxvirus vectors, adeno-associated virus vectors, and HVJ vectors (see Adolph “Viral Genome Methods” CRC Press, Florida (1996)), or coating with carrier beads such as colloidal gold particles (WO 93/17706, and such). However, so long as the antibodies are expressed in vivo and their activities are exercised, any method can be used for administration. Preferably, a sufficient dose can be administered by a suitable parenteral route (such as injecting or infusing intravenously, intraperitoneally, subcutaneously, intradermally, intramuscularly, into adipose tissues or mammary glands; inhalation; gas-driven particle bombardment (using electron gun and such); or mucosal route such as nasal drops). Alternatively, the genes encoding the antibodies of the present invention may be administered into blood cells, bone marrow cells, and such ex vivo using liposome transfection, particle bombardment (U.S. Pat. No. 4,945,050), or viral infection, and the cells may be reintroduced into patients. Any gene encoding an antibody of the present invention may be used in gene therapy, and its examples include genes comprising nucleotide sequences encoding the CDRs of Q1, Q31, Q64, Q85, Q153, Q354, Q360, Q405, Q458, Q460, Q499, J232, J259, J268, J300, J321, J326, J327, J339, J344, J346, J142, L2, L45, L248, L324, L334, L377, L404, L406, L408, and L180 described above.

The present invention also provides methods for preventing and/or treating bleeding, diseases accompanying bleeding, and/or diseases caused by bleeding, such methods comprising the step of administering the antibodies or compositions of the present invention. The antibodies or compositions can be administered, for example, by the above-mentioned methods.

Furthermore, the present invention provides kits to be used for the above-mentioned methods, such kits comprising at least an antibody or composition of the present invention. In addition, the kits may include, packaged therewith, a syringe, injection needle, pharmaceutically acceptable medium, alcohol-soaked cotton, adhesive bandage, instructions describing the method of use, and the like.

The present invention also relates to use of a multispecific antigen-binding molecule, a bispecific antibody, or a composition of the present invention in the manufacture of an agent for preventing and/or treating bleeding, a disease accompanying bleeding, or a disease caused by bleeding.

Furthermore, the present invention relates to a multispecific antigen-binding molecule, a bispecific antibody, or a composition of the present invention for preventing and/or treating bleeding, a disease accompanying bleeding, or a disease caused by bleeding.

All prior art references cited herein are incorporated by reference into this description.

### EXAMPLES

Herein below, the present invention will be specifically described with reference to the Examples, but it is not to be construed as being limited thereto.

### [Example 1] Production of Bispecific Antibodies Having F.Xa Generation-Promoting Activity

In WO 2006/109592, hA69-KQ/hB26-PF/hAL-AQ was obtained as a bispecific antibody having an activity of functionally substituting for F.VIII. However, there was the possibility that this antibody has an inhibiting action on the reaction in which F.IXa activates F.X using F.VIIIa as a cofactor.

As shown in FIG. 1, antibodies that bind to F.IX/F.IXa or F.X may inhibit the formation of the F.IXa-F.VIIIa complex (Factor Xase (F.Xase)), or inhibit F.Xase activity (activation of EX). Hereafter, inhibition of F.Xase formation and/or action of inhibiting F.Xase activity will be mentioned as F.Xase inhibitory action. F.Xase inhibitory action is the inhibition of a coagulation reaction in which F.VIIIa serves as the cofactor, which may suppress the remaining F.VIII function in a patient or the function of the administered F.VIII formulation. Therefore, it is desirable that F.Xa generation-promoting activity, which is the objective of the bispecific antibody, is high, while F.Xase inhibitory action is low. In particular, for patients maintaining F.VIII function and patients receiving treatment with a F.VIII formulation, it is more desirable that F.Xa generation-promoting activity and F.Xase inhibitory action are separated as much as possible.

However, F.Xase inhibitory action is due to the binding to the antigen (F.IXa and/or F.X), which is fundamental property of the antibody. On the other hand, a bispecific antibody having F.Xa generation-promoting action (functionally substituting for F.VIII) also needs to bind to the antigens (F.IXa and F.X). Therefore, it is predicted that it is extremely difficult to obtain bispecific antibodies that do not have an F.Xase inhibitory action but have an F.Xa generation-promoting activity (functionally substituting for F.VIII). Similarly, it is predicted that it is extremely difficult to decrease an F.Xase inhibitory action while increasing the target F.Xa generation-promoting activity by introducing amino acid substitutions in a bispecific antibody.

The present inventors prepared genes for approximately 200 types of antibodies against human F.IXa and human F.X, respectively, using a method known to those skilled in the art, which is the method of obtaining antibody genes from antibody-producing cells of animals immunized with an antigen (human F.IXa or human F.X), and introducing amino acid substitutions, when necessary. Each antibody gene was incorporated into an animal cell expression vector. 40,000 or more bispecific antibodies as anti-F.IXa antibody and anti-F.X antibody combinations were transiently expressed by simultaneously transfecting the anti-human F.IXa antibody H chain expression vector, the anti-human F.X antibody H chain expression vector, and the commonly shared antibody L chain expression vector into mammalian cells such as HEK293H cells. As a comparative control, bispecific antibody hA69-KQ/hB26-PF/hAL-AQ (SEQ ID NOs: 165/166/167) described in WO 2006/109592 was prepared.

Since the mutations mentioned in WO 2006/106905 or WO 1996/027011 were introduced into the CH3 domain of each H chain, it was thought that bispecific antibodies were mainly expressed. Antibodies in the cell culture supernatant were purified by a method known to those skilled in the art using Protein A.

The present inventors measured the F.Xa generation-promoting activity of these antibodies by the method described below. All reactions were performed at room temperature.

Five μL of antibody solution diluted with Tris-buffered saline containing 0.1% bovine serum albumin (hereafter referred to as TBSB) was mixed with 2.5 μL of 27 ng/mL Human Factor IXa beta (Enzyme Research Laboratories) and 2.5 μL of 6 IU/mL of human blood coagulation factor IX (Novact (registered trademark) M (Kaketsuken)), and then incubated in a 384-well plate at room temperature for 30 minutes.

The enzyme reaction in this mixed solution was initiated by adding 5 μL of 24.7 μg/mL of Human Factor X (Enzyme Research Laboratories), and ten minutes later, 5 μL of 0.5 M EDTA was added to stop the reaction. The coloring reaction was initiated by adding 5 μL of coloring substrate solution. After a 50-minute coloring reaction, the change in absorbance at 405 nm was measured using the SpectraMax 340PC³⁸⁴ (Molecular Devices). F.Xa generation-promoting activity was indicated as the value obtained by subtracting the absorbance of the antibody-free reaction solution from the absorbance of the antibody-supplemented reaction solution.

TBCP (TBSB containing 93.75 μM synthetic phospholipid solution (SYSMEX CO.), 7.5 mM CaCl₂), and 1.5 mM MgCl₂) was used as the solvent for Human Factor IXa, Novact (registered trademark) M, and Human Factor X. A coloring substrate solution N-benzoyl-L-isoleucyl-L-glutamyl-glycyl-L-arginine-p-nitroaniline hydrochloride (S-2222™; CHROMOGENIX) was dissolved in purified water at 1.47 mg/mL, and then used in this assay.

To evaluate the F.Xase inhibitory action of the antibodies, the present inventors measured the effects on F.X activation by F.IXa in the presence of F.VIIIa using the following method. All reactions were performed at room temperature.

Five μL of antibody solution diluted with Tris-buffered saline containing 0.1% bovine serum albumin (hereafter referred to as TBSB) was mixed with 2.5 μL of 80.9 ng/mL Human Factor IXa beta (Enzyme Research Laboratories), and then incubated in a 384-well plate at room temperature for 30 minutes.

2.5 μL of 1.8 IU/mL of F.VIIIa (production method will be descried later) was further added, and 30 seconds later, the enzyme reaction in this mixed solution was initiated by adding 5 μL of 24.7 μg/mL of Human Factor X (Enzyme Research Laboratories). Six minutes later, 5 μL of 0.5 M EDTA was added to stop the reaction. The coloring reaction was initiated by adding 5 μL of coloring substrate solution. After a 14-minute coloring reaction, the change in absorbance at 405 nm was measured using the SpectraMax 340PC³⁸⁴ (Molecular Devices). F.Xase inhibitory action of an antibody was indicated as the value obtained by subtracting the absorbance of the antibody-free reaction solution from the absorbance of the antibody-supplemented reaction solution.

F.VIIIa was prepared by mixing 5.4 IU/mL of Kogenate (registered trademark) FS (Bayer HealthCare) and 1.11 μg/mL of Human alpha Thrombin (Enzyme Research Laboratories) at a volume ratio of 1:1, incubating at room temperature for one minute, and then adding 7.5 U/mL of Hirudin (Merck KgaA) at a quantity that is half the volume of the mixture solution. The prepared solution was defined as 1.8 IU/mL of FVIIIa, and one minute after addition of Hirudin, this was used for assays.

TBCP (TBSB containing 93.75 μM phospholipid solution (SYSMEX CO.), 7.5 mM CaCl₂), and 1.5 mM MgCl₂) was used for the solvent for Human Factor IXa, Human Factor X, Kogenate (registered trademark) FS, Human alpha Thrombin, and Hirudin. A coloring substrate solution S-2222™ (CHROMOGENIX) was dissolved in purified water at 1.47 mg/mL, and then used in this assay.

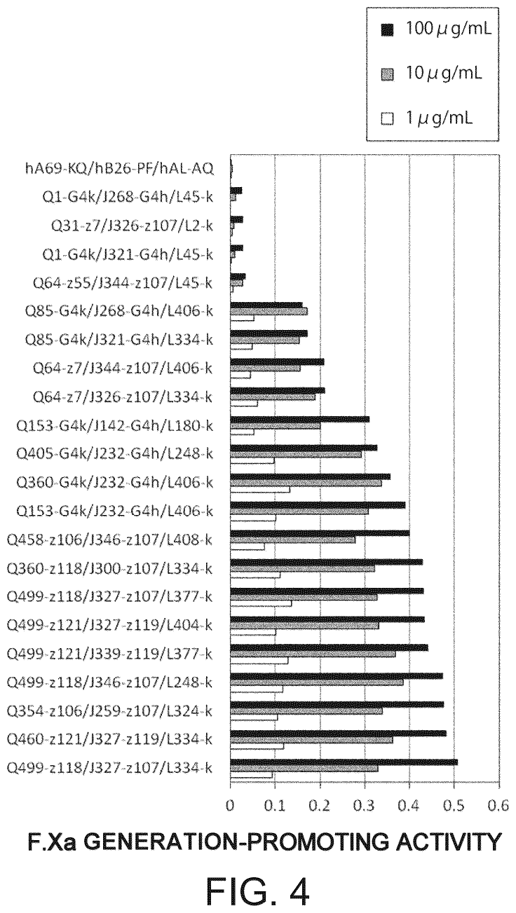

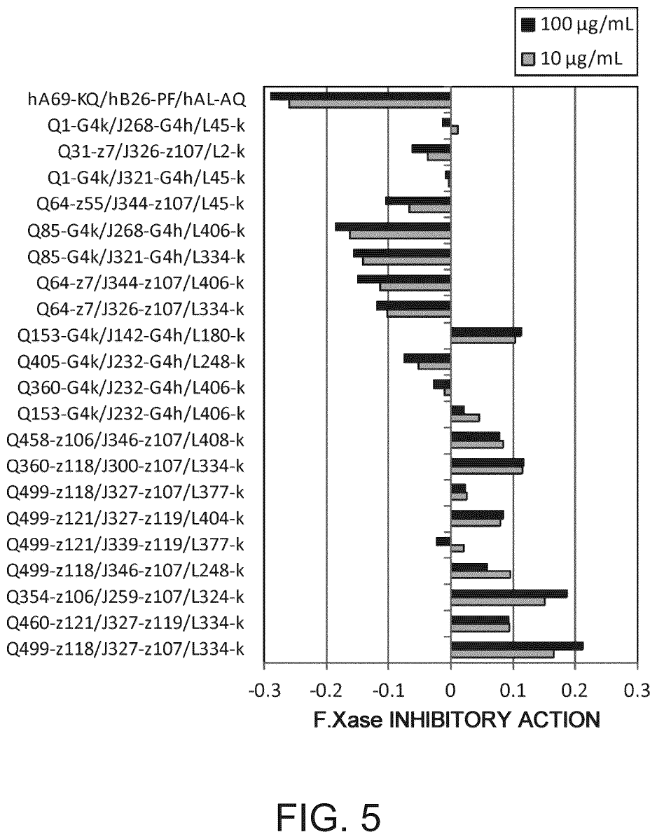

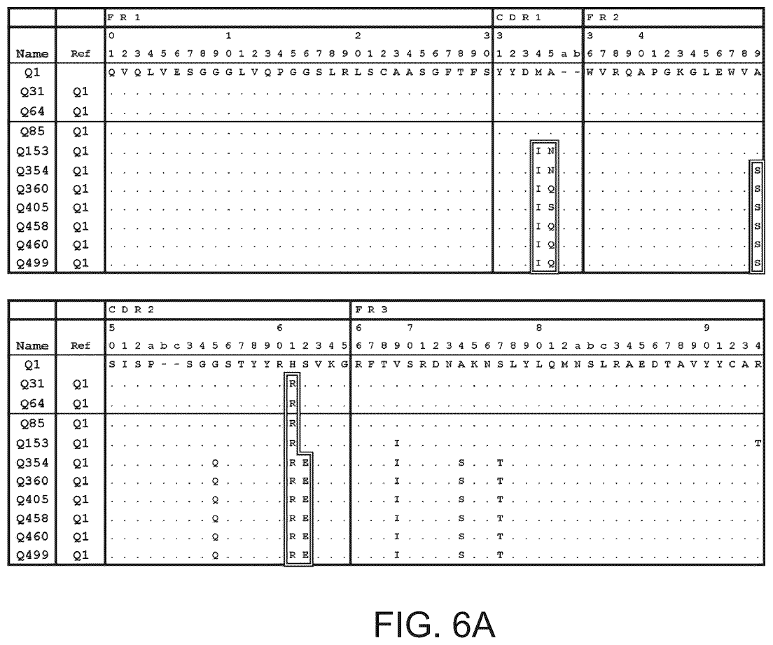

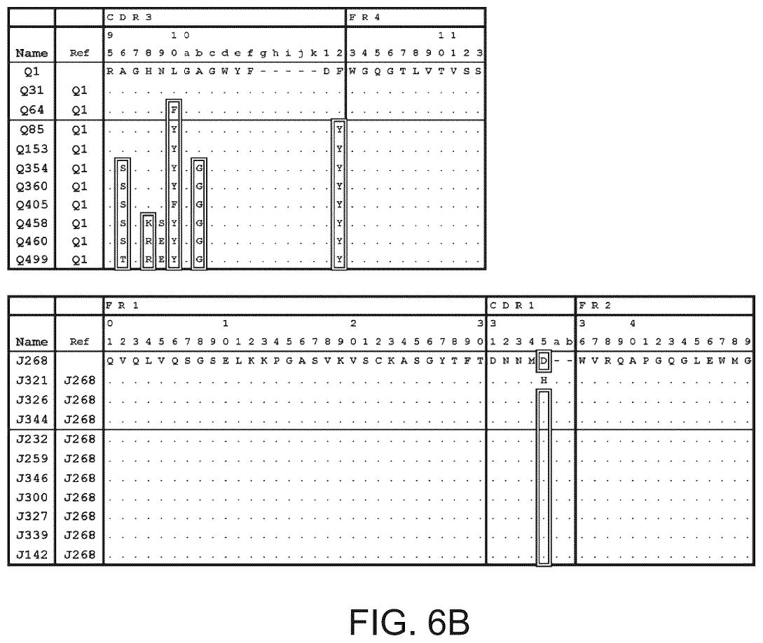

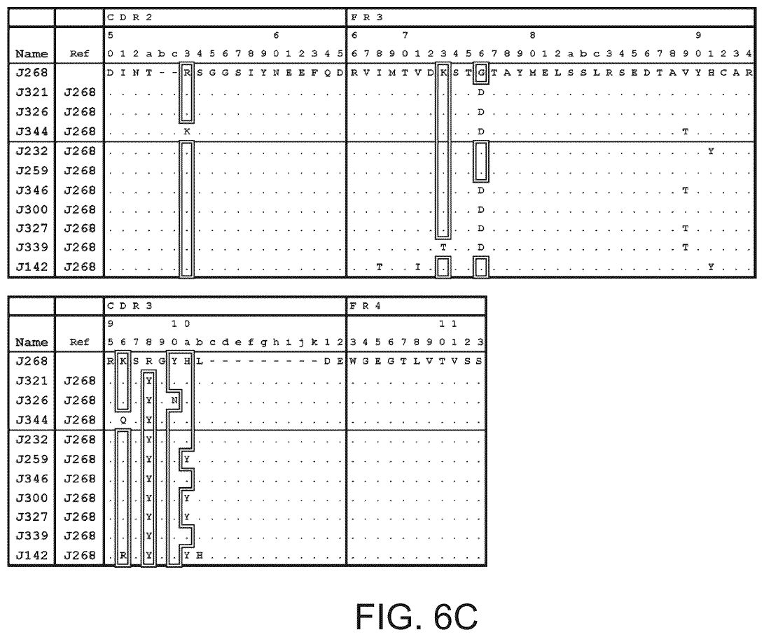

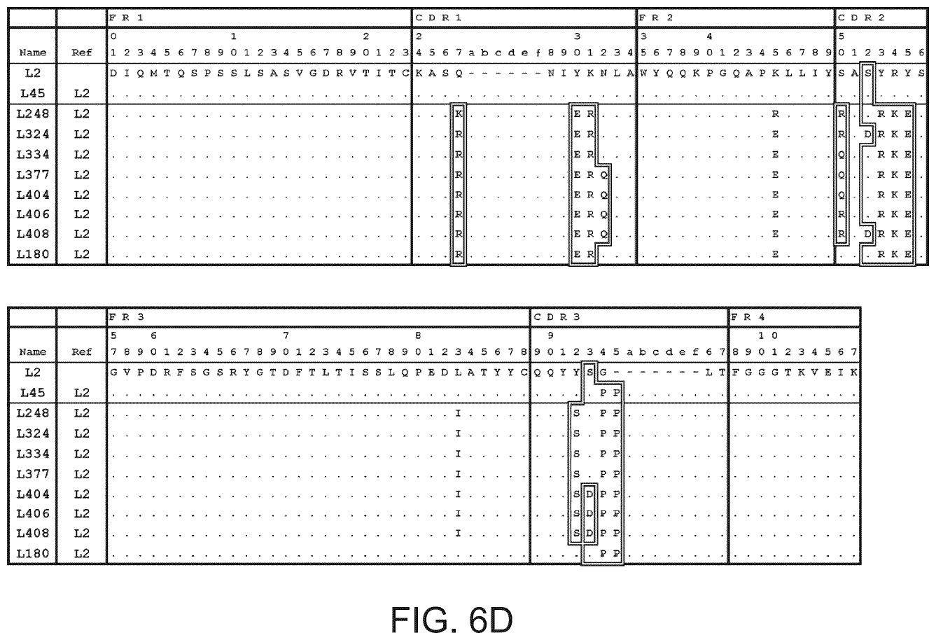

The F.Xa generation-promoting activities of each of the bispecific antibodies are indicated in FIGS. 3 and 4, and the F.Xase inhibitory actions of each of the bispecific antibodies are indicated in FIG. 5. Various amino acid substitutions that increase the F.Xa generation-promoting activity have been found, but as expected, most of the amino acid substitutions that increase the F.Xa generation-promoting activity increased F.Xase inhibitory action as well, and suppressing F.Xase inhibitory action while increasing F.Xa generation-promoting activity was very difficult.

Under such circumstances, the inventors of the present application obtained Q1-G4k/J268-G4h/L45-k, Q1-G4k/J321-G4h/L45-k, Q31-z7/J326-z107/L2-k, Q64-z55/J344-z107/L45-k as bispecific antibodies with a high F.Xa generation-promoting activity and a low F.Xase inhibitory action. In addition, Q1-G4k (SEQ ID NO: 1), Q31-z7 (SEQ ID NO: 2), and Q64-z55 (SEQ ID NO: 3) were obtained as anti-human F.IXa antibody H chains, J268-G4h (SEQ ID NO: 4), J321-G4h (SEQ ID NO: 5), J326-z107 (SEQ ID NO: 6), and J344-z107 (SEQ ID NO: 7) were obtained as prototype anti-human F.X antibody H chains, and L2-k (SEQ ID NO: 8) and L45-k (SEQ ID NO: 9) were obtained as prototype commonly shared antibody L chains. The character before the hyphen in the sequence name indicates the variable region and the character after the hyphen indicates the constant region. Each bispecific antibody name is indicated by listing the sequence names of each chain to be transfected.

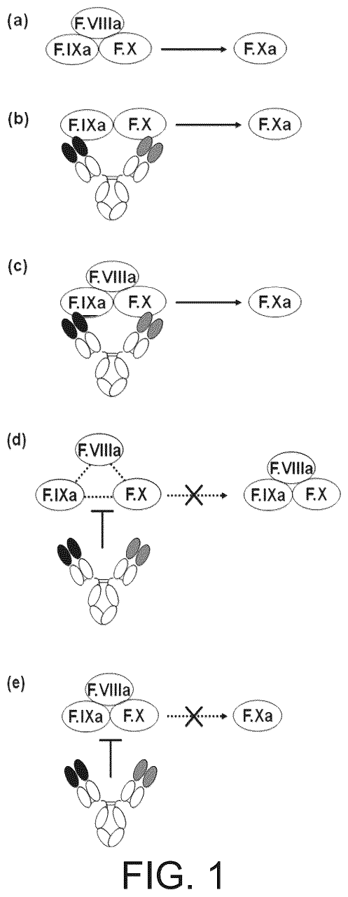

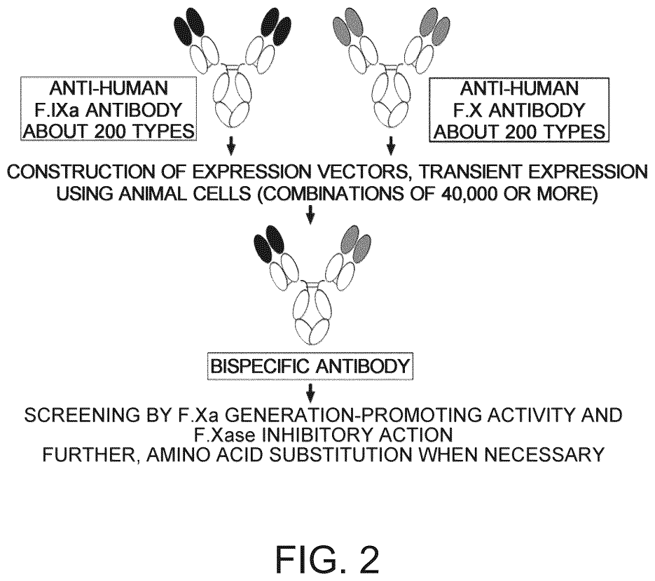

Most of the bispecific antibodies having F.Xa generation-promoting activity close to that of hA69-KQ/hB26-PF/hAL-AQ had high F.Xase inhibitory action as expected, but these bispecific antibodies (Q1-G4k/J268-G4h/L45-k, Q1-G4k/J321-G4h/L45-k, Q31-z7/J326-z107/L2-k, Q64-z55/J344-z107/L45-k) were found to have higher F.Xa generation-promoting activity and lower F.Xase inhibitory action than hA69-KQ/hB26-PF/hAL-AQ described in WO 2006/109592. The present inventors conducted examinations to further increase the F.Xa generation-promoting activity and reduce the F.Xase inhibitory action using these four antibodies as prototype antibodies. Screening of bispecific antibodies that increase F.Xa generation-promoting activity and reduce F.Xase inhibitory action is indicated in FIG. 2.

### [Example 2] Production of Modified Antibodies

The present inventors introduced various combinations of amino acid mutations that affect the F.Xa generation-promoting activities and F.Xase inhibitory actions found in Example 1 to each of the chains of the prototype antibodies by a method known to those skilled in the art such as PCR for introducing mutations and evaluated the combinations of modified chains on a large scale to screen for amino acid substitutions that will further increase the F.Xa generation-promoting activities and reduce the F.Xase inhibitory actions of the four prototype antibodies.

Each of the modified bispecific antibodies with amino acid substitutions were expressed transiently and purified by methods similar to those for the prototype antibodies. The F.Xa generation-promoting activities of the antibodies were measured using the following method. All reactions were performed at room temperature.

Five μL of antibody solution diluted with Tris-buffered saline containing 0.1% bovine serum albumin (hereafter referred to as TBSB) was mixed with 2.5 μL of 27 ng/mL Human Factor IXa beta (Enzyme Research Laboratories) and 2.5 μL of 6 IU/mL of Novact (registered trademark) M (Kaketsuken), and then incubated in a 384-well plate at room temperature for 30 minutes.

The enzyme reaction in this mixed solution was initiated by adding 5 μL of 24.7 μg/mL of Human Factor X (Enzyme Research Laboratories), and two minutes later, 5 μL of 0.5 M EDTA was added to stop the reaction. The coloring reaction was initiated by adding 5 μL of coloring substrate solution. After a 20-minute coloring reaction, the change in absorbance at 405 nm was measured using the SpectraMax 340PC³⁸⁴ (Molecular Devices). F.Xa generation-promoting activity was indicated as the value obtained by subtracting the absorbance of the antibody-free reaction solution from the absorbance of the antibody-supplemented reaction solution.

TBCP (TBSB containing 93.75 μM phospholipid solution (SYSMEX CO.), 7.5 mM CaCl₂), and 1.5 mM MgCl₂) was used as the solvent for Human Factor IXa, Novact (registered trademark) M, and Human Factor X. A coloring substrate solution S-2222™ (CHROMOGENIX) was dissolved in purified water at 1.47 mg/mL, and then used in this assay.

F.Xase inhibitory actions of the antibodies were also evaluated by previously described methods.

The F.Xa generation-promoting activities of each of the modified bispecific antibodies are indicated in FIG. 4, and the F.Xase inhibitory actions of each of the bispecific antibodies are indicated in FIG. 5.

The inventors of the present application obtained Q85-G4k/J268-G4h/L406-k, Q85-G4k/J321-G4h/L334-k, Q64-z7/J344-z107/L406-k, and Q64-z7/J326-z107/L334-k as bispecific antibodies with a high F.Xa generation-promoting activity and a low F.Xase inhibitory action. In addition, they discovered Q64-z7 (SEQ ID NO: 10) and Q85-G4k (SEQ ID NO: 11) as the anti-human F.IXa antibody H chain, and L334-k (SEQ ID NO: 30) and L406-k (SEQ ID NO: 33) as the commonly shared antibody L chains with increased F.Xa generation-promoting activity. Though F.Xase inhibitory actions increased slightly, F.Xa generation-promoting activity increased greatly in Q85-G4k/J268-G4h/L406-k, Q85-G4k/J321-G4h/L334-k, Q64-z7/J344-z107/L406-k, and Q64-z7/J326-z107/L334-k. Since these modified antibodies have very large F.Xa generation-promoting activities compared to increase in F.Xase inhibitory actions, the F.Xa generation-promoting activity and the F.Xase inhibitory action could further be separated compared to the prototype antibodies. This way, combinations that suppress the F.Xase inhibitory action and increase the F.Xa generation-promoting activity were discovered.

While a higher F.Xa generation-promoting activity is preferred for the discovered prototype antibodies to functionally substitute for F.VIII by bispecific antibodies, lower F.Xase inhibitory action was considered favorable to clinically use for patients maintaining F.VIII functions or patients receiving treatment with F.VIII formulations. Therefore, further modifications were performed to produce bispecific antibodies in which F.Xase inhibitory action is not increased while F.Xa generation-promoting activity is further increased.

As a result, Q153-G4k/J232-G4h/L406-k, Q354-z106/J259-z107/L324-k, Q360-G4k/J232-G4h/L406-k, Q360-z118/J300-z107/L334-k, Q405-G4k/J232-G4h/L248-k, Q458-z106/J346-z107/L408-k, Q460-z121/J327-z119/L334-k, Q499-z118/J327-z107/L334-k, Q499-z118/J327-z107/L377-k, Q499-z118/J346-z107/L248-k, Q499-z121/J327-z119/L404-k, Q499-z121/J339-z119/L377-k, and Q153-G4k/J142-G4h/L180-k were obtained as bispecific antibodies with a high F.Xa generation-promoting activity and a low F.Xase inhibitory action. In addition, the Inventors discovered Q153-G4k (SEQ ID NO: 12), Q354-z106 (SEQ ID NO: 13), Q360-G4k (SEQ ID NO: 14), Q360-z118 (SEQ ID NO: 15), Q405-G4k (SEQ ID NO: 16), Q458-z106 (SEQ ID NO: 17), Q460-z121 (SEQ ID NO: 18), Q499-z118 (SEQ ID NO: 19), and Q499-z121 (SEQ ID NO: 20) as the anti-human F.IXa antibody H chain, J232-G4h (SEQ ID NO: 21), J259-z107 (SEQ ID NO: 22), J300-z107 (SEQ ID NO: 23), J327-z107 (SEQ ID NO: 24), J327-z119 (SEQ ID NO: 25), J339-z119 (SEQ ID NO: 26), J346-z107 (SEQ ID NO: 27), J142-G4h (SEQ ID NO: 170) as the anti-human F.X antibody H chains with increased F.Xa generation-promoting activity, and L248-k (SEQ ID NO: 28), L324-k (SEQ ID NO: 29), L377-k (SEQ ID NO: 31), L404-k (SEQ ID NO: 32), L408-k (SEQ ID NO: 34), and L180-k (SEQ ID NO: 171) as the commonly shared antibody L chains.

Since these antibodies have very high F.Xa generation-promoting activities while having suppressed F.Xase inhibitory actions, they may have very useful properties for patients maintaining an F.VIII function and patients receiving treatment with F.VIII formulations. Since antibodies generally have long half-lives, and can be administered subcutaneously, these bispecific antibodies may be of great value to hemophilia A patients, when compared to existing replacement therapy by intravenous administration of existing F.VIII formulations for hemophilia A.

Sequence comparisons of the variable regions of each of the chains used in Example 1 and Example 2 are shown in FIGS. 6A to D. For example, to enhance the F.Xa generation-promoting activity of a bispecific antibody, the following amino acids were found to be important: in the anti-human F.IXa antibody H chain, isoleucine at position 34, asparagine, glutamine, or serine at position 35, serine at position 49, arginine at position 61, glutamic acid at position 62, serine or threonine at position 96, lysine or arginine at position 98, serine or glutamic acid at position 99, phenylalanine or tyrosine at position 100, glycine at position 100b, tyrosine at position 102, and such; in the anti-human F.X antibody H chain, aspartic acid at position 35, arginine at position 53, lysine at position 73, glycine at position 76, lysine or arginine at position 96, tyrosine at position 98, tyrosine at position 100, histidine at position 100a, and such; in the commonly shared antibody L chain, lysine or arginine at position 27, glutamic acid at position 30, arginine at position 31, glutamine at position 32, arginine or glutamine at position 50, serine at position 52, arginine at position 53, lysine at position 54, glutamic acid at position 55, serine at position 92, serine at position 93, proline at position 94, proline at position 95, and such (the variable region amino acids are numbered by Kabat numbering (Kabat E A et al. 1991. Sequences of Proteins of Immunological Interest. NIH)).

### INDUSTRIAL APPLICABILITY

The present invention provides multispecific antigen-binding molecules having a high activity of functionally substituting for F.VIII, which are antibodies that recognize both an enzyme and its substrate. Furthermore, the present invention provides multispecific antigen-binding molecules with a high activity of functionally substituting for F.VIII and a low F.Xase inhibitory action, which are antibodies that recognize both an enzyme and its substrate.

Since humanized antibodies may generally have high stability in blood and low immunogenicity, multispecific antibodies of the present invention may be very promising as pharmaceuticals.

1. A multispecific antigen-binding molecule that functionally substitutes for blood coagulation factor VIII, which comprises a first antigen-binding site that recognizes blood coagulation factor IX and/or activated blood coagulation factor IX and a second antigen-binding site that recognizes blood coagulation factor X, wherein the functional substitution for blood coagulation factor VIII results from an activated blood coagulation factor X (F.Xa) generation-promoting activity higher than the activity of a bispecific antibody (hA69-KQ/hB26-PF/hAL-AQ) which comprises an H chain comprising SEQ ID NOs: 165 and 166, and a commonly shared L chain comprising SEQ ID NO: 167.

2. The multispecific antigen-binding molecule of claim 1, which comprises a first polypeptide comprising a first antigen-binding site that recognizes blood coagulation factor IX and/or activated blood coagulation factor IX and a third polypeptide comprising a third antigen-binding site that recognizes blood coagulation factor IX and/or activated blood coagulation factor IX, as well as a second polypeptide comprising a second antigen-binding site that recognizes blood coagulation factor X and a fourth polypeptide comprising a fourth antigen-binding site that recognizes blood coagulation factor X.

3. The multispecific antigen-binding molecule of claim 2, wherein the first polypeptide and the third polypeptide each comprises an antigen-binding site of an H chain or L chain of an antibody against blood coagulation factor IX or activated blood coagulation factor IX, respectively; and the second polypeptide and the fourth polypeptide each comprises an antigen-binding site of an H chain or L chain of an antibody against blood coagulation factor X, respectively.

4. The multispecific antigen-binding molecule of claim 3, wherein the antigen-binding site of the first polypeptide comprises an antigen-binding site which comprises H chain CDRs consisting of any one of the amino acid sequences selected from the following (a1) to (a11), or an antigen-binding site functionally equivalent thereto, and the antigen-binding site of the second polypeptide comprises an antigen-binding site which comprises H chain CDRs consisting of any one of the amino acid sequences selected from the following (b1) to (b11), or an antigen-binding site functionally equivalent thereto:
(a1) an antigen-binding site comprising an H chain CDR 1, 2, and 3 amino acid sequences of SEQ ID NOs: 75, 76, and 77 (H chain CDRs of Q1), respectively;
(a2) an antigen-binding site comprising an H chain CDR 1, 2, and 3 amino acid sequences of SEQ ID NOs: 78, 79, and 80 (H chain CDRs of Q31), respectively;
(a3) an antigen-binding site comprising an H chain CDR 1, 2, and 3 amino acid sequences of SEQ ID NOs: 81, 82, and 83 (H chain CDRs of Q64), respectively;
(a4) an antigen-binding site comprising an H chain CDR 1, 2, and 3 amino acid sequences of SEQ ID NOs: 84, 85, and 86 (H chain CDRs of Q85), respectively;
(a5) an antigen-binding site comprising the H chain CDR 1, 2, and 3 amino acid sequences of SEQ ID NOs: 87, 88, and 89 (H chain CDRs of Q153), respectively;
(a6) an antigen-binding site comprising an H chain CDR 1, 2, and 3 amino acid sequences of SEQ ID NOs: 90, 91, and 92 (H chain CDRs of Q354), respectively;
(a7) an antigen-binding site comprising the H chain CDR 1, 2, and 3 amino acid sequences of SEQ ID NOs: 93, 94, and 95 (H chain CDRs of Q360), respectively;
(a8) an antigen-binding site comprising the of H chain CDR 1, 2, and 3 amino acid sequences of SEQ ID NOs: 96, 97, and 98 (H chain CDRs of Q405), respectively;
(a9) an antigen-binding site comprising an H chain CDR 1, 2, and 3 amino acid sequences of SEQ ID NOs: 99, 100, and 101 (H chain CDRs of Q458), respectively;
(a10) an antigen-binding site comprising an H chain CDR 1, 2, and 3 amino acid sequences of SEQ ID NOs: 102, 103, and 104 (H chain CDRs of Q460), respectively;
(a11) an antigen-binding site comprising an H chain CDR 1, 2, and 3 amino acid sequences of SEQ ID NOs: 105, 106, and 107 (H chain CDRs of Q499), respectively;
(b1) an antigen-binding site comprising an H chain CDR 1, 2, and 3 amino acid sequences of SEQ ID NOs: 108, 109, and 110 (H chain CDRs of J232), respectively;
(b2) an antigen-binding site comprising an H chain CDR 1, 2, and 3 amino acid sequences of SEQ ID NOs: 111, 112, and 113 (H chain CDRs of J259), respectively;
(b3) an antigen-binding site comprising an H chain CDR 1, 2, and 3 amino acid sequences of SEQ ID NOs: 114, 115, and 116 (H chain CDRs of J268), respectively;
(b4) an antigen-binding site comprising an H chain CDR 1, 2, and 3 amino acid sequences of SEQ ID NOs: 117, 118, and 119 (H chain CDRs of J300), respectively;
(b5) an antigen-binding site comprising an H chain CDR 1, 2, and 3 amino acid sequences of SEQ ID NOs: 120, 121, and 122 (H chain CDRs of J321), respectively;
(b6) an antigen-binding site comprising the H chain CDR 1, 2, and 3 amino acid sequences of SEQ ID NOs: 123, 124, and 125 (H chain CDRs of J326), respectively;
(b7) an antigen-binding site comprising an H chain CDR 1, 2, and 3 amino acid sequences of SEQ ID NOs: 126, 127, and 128 (H chain CDRs of J327), respectively;
(b8) an antigen-binding site comprising an H chain CDR 1, 2, and 3 amino acid sequences of SEQ ID NOs: 129, 130, and 131 (H chain CDRs of J339), respectively;
(b9) an antigen-binding site comprising an H chain CDR 1, 2, and 3 amino acid sequences of SEQ ID NOs: 132, 133, and 134 (H chain CDRs of J344), respectively;
(b10) an antigen-binding site comprising an H chain CDR 1, 2, and 3 amino acid sequences of SEQ ID NOs: 135, 136, and 137 (H chain CDRs of J346), respectively; and
(b11) an antigen-binding site comprising an H chain CDR 1, 2, and 3 amino acid sequences of SEQ ID NOs: 174, 175, and 176 (H chain CDRs of J142), respectively.

5. The multispecific antigen-binding molecule of claim 3, wherein the antigen-binding site of the first polypeptide comprises an antigen-binding site which comprises an H chain variable region consisting of any one of the amino acid sequences selected from the following (a1) to (a11), or an antigen-binding site functionally equivalent thereto, and the antigen-binding site of the second polypeptide comprises an antigen-binding site which comprises an H chain variable region consisting of any one of the amino acid sequences selected from the following (b1) to (b11), or an antigen-binding site functionally equivalent thereto:
(a1) an antigen-binding site comprising an H chain variable region amino acid sequence of SEQ ID NO: 35 (H chain variable region of Q1);
(a2) an antigen-binding site comprising an H chain variable region amino acid sequence of SEQ ID NO: 36 (H chain variable region of Q31);
(a3) an antigen-binding site comprising an H chain variable region amino acid sequence of SEQ ID NO: 37 (H chain variable region of Q1);
(a4) an antigen-binding site comprising an H chain variable region amino acid sequence of SEQ ID NO: 38 (H chain variable region of Q85);
(a5) an antigen-binding site comprising an H chain variable region amino acid sequence of SEQ ID NO: 39 (H chain variable region of Q153);
(a6) an antigen-binding site comprising an H chain variable region amino acid sequence of SEQ ID NO: 40 (H chain variable region of Q354);
(a7) an antigen-binding site comprising an H chain variable region amino acid sequence of SEQ ID NO: 41 (H chain variable region of Q360);
(a8) an antigen-binding site comprising an H chain variable region amino acid sequence of SEQ ID NO: 42 (H chain variable region of Q405);
(a9) an antigen-binding site comprising an H chain variable region amino acid sequence of SEQ ID NO: 43 (H chain variable region of Q458);
(a10) an antigen-binding site comprising an H chain variable region amino acid sequence of SEQ ID NO: 44 (H chain variable region of Q460);
(a11) an antigen-binding site comprising an H chain variable region amino acid sequence of SEQ ID NO: 45 (H chain variable region of Q499);
(b1) an antigen-binding site comprising an H chain variable region amino acid sequence of SEQ ID NO: 46 (H chain variable region of J232);
(b2) an antigen-binding site comprising an H chain variable region amino acid sequence of SEQ ID NO: 47 (H chain variable region of J259);
(b3) an antigen-binding site comprising an H chain variable region amino acid sequence of SEQ ID NO: 48 (H chain variable region of J268);
(b4) an antigen-binding site comprising an H chain variable region amino acid sequence of SEQ ID NO: 49 (H chain variable region of J300);
(b5) an antigen-binding site comprising an H chain variable region amino acid sequence of SEQ ID NO: 50 (H chain variable region of J321);
(b6) an antigen-binding site comprising an H chain variable region amino acid sequence of SEQ ID NO: 51 (H chain variable region of J326);
(b7) an antigen-binding site comprising an H chain variable region amino acid sequence of SEQ ID NO: 52 (H chain variable region of J327);
(b8) an antigen-binding site comprising an H chain variable region amino acid sequence of SEQ ID NO: 53 (H chain variable region of J339);
(b9) an antigen-binding site comprising an H chain variable region amino acid sequence of SEQ ID NO: 54 (H chain variable region of J344);
(b10) an antigen-binding site comprising an H chain variable region amino acid sequence of SEQ ID NO: 55 (H chain variable region of J346); and
(b11) an antigen-binding site comprising an H chain variable region amino acid sequence of SEQ ID NO: 172 (H chain variable region of J142).

6. The multispecific antigen-binding molecule of claim 3, wherein the antigen-binding sites included in the third polypeptide and the fourth polypeptide comprise an antigen-binding site which comprises L chain CDRs consisting of any one of the amino acid sequences selected from the following (c1) to (c10), or an antigen-binding site functionally equivalent thereto:
(c1) an antigen-binding site comprising an L chain CDR1, 2, and 3 amino acid sequences of SEQ ID NOs: 138, 139, and 140 (L chain CDR of L2), respectively;
(c2) an antigen-binding site comprising an L chain CDR1, 2, and 3 amino acid sequences of SEQ ID NOs: 141, 142, and 143 (L chain CDR of L45), respectively;
(c3) an antigen-binding site comprising an L chain CDR1, 2, and 3 amino acid sequences of SEQ ID NOs: 144, 145, and 146 (L chain CDR of L248), respectively;
(c4) an antigen-binding site comprising an L chain CDR1, 2, and 3 amino acid sequences of SEQ ID NOs: 147, 148, and 149 (L chain CDR of L324), respectively;
(c5) an antigen-binding site comprising an L chain CDR1, 2, and 3 amino acid sequences of SEQ ID NOs: 150, 151, and 152 (L chain CDR of L334), respectively;
(c6) an antigen-binding site comprising an L chain CDR1, 2, and 3 amino acid sequences of SEQ ID NOs: 153, 154, and 155 (L chain CDR of L377), respectively;
(c7) an antigen-binding site comprising an L chain CDR1, 2, and 3 amino acid sequences of SEQ ID NOs: 156, 157, and 158 (L chain CDR of L404), respectively;
(c8) an antigen-binding site comprising an L chain CDR1, 2, and 3 amino acid sequences of SEQ ID NOs: 159, 160, and 161 (L chain CDR of L406), respectively;
(c9) an antigen-binding site comprising an L chain CDR1, 2, and 3 amino acid sequences of SEQ ID NOs: 137, 138, and 139 (L chain CDR of L408), respectively; and
(c10) an antigen-binding site comprising an L chain CDR1, 2, and 3 amino acid sequences of SEQ ID NOs: 177, 178, and 179 (L chain CDR of L180), respectively.

7. The multispecific antigen-binding molecule of claim 3, wherein the antigen-binding sites included in the third polypeptide and the fourth polypeptide comprise an antigen-binding site which comprises an L chain variable region consisting of any one of the amino acid sequences selected from the following (c1) to (c10), or an antigen-binding site functionally equivalent thereto:
(c1) an antigen-binding site comprising an L chain variable region amino acid sequence of SEQ ID NO: 56 (L chain variable region of L2);
(c2) an antigen-binding site comprising an L chain variable region amino acid sequence of SEQ ID NO: 57 (L chain variable region of L45);
(c3) an antigen-binding site comprising an L chain variable region amino acid sequence of SEQ ID NO: 58 (L chain variable region of L248);
(c4) an antigen-binding site comprising an L chain variable region amino acid sequence of SEQ ID NO: 59 (L chain variable region of L324);
(c5) an antigen-binding site comprising an L chain variable region amino acid sequence of SEQ ID NO: 60 (L chain variable region of L334);
(c6) an antigen-binding site comprising an L chain variable region amino acid sequence of SEQ ID NO: 61 (L chain variable region of L377);
(c7) an antigen-binding site comprising an L chain variable region amino acid sequence of SEQ ID NO: 62 (L chain variable region of L404);
(c8) an antigen-binding site comprising an L chain variable region amino acid sequence of SEQ ID NO: 63 (L chain variable region of L406);
(c9) an antigen-binding site comprising an L chain variable region amino acid sequence of SEQ ID NO: 64 (L chain variable region of L408); and
(c10) an antigen-binding site comprising an L chain variable region amino acid sequence of SEQ ID NO: 173 (L chain variable region of L180).

8. The multispecific antigen-binding molecule of claim 3, wherein the first and second polypeptides further comprise an antibody H chain constant region, and the third and fourth polypeptides comprise an antibody L chain constant region.

9. The multispecific antigen-binding molecule of claim 3, wherein the first and second polypeptides comprise an antibody H chain constant region, and the third and fourth polypeptides comprise an antibody L chain constant region, and wherein the third polypeptide and the fourth polypeptide are a commonly shared L chain.

10. The multispecific antigen-binding molecule of claim 8, wherein the first polypeptide comprises any one antibody H chain selected from the following (a1) to (a14), the second polypeptide comprises any one antibody H chain selected from the following (b1) to (b12), and the third polypeptide and the fourth polypeptide comprise any one antibody L chain selected from the following (c1) to (c10):
(a1) an antibody H chain consisting of the amino acid sequence of SEQ ID NO: 1 (Q1-G4k);
(a2) an antibody H chain consisting of the amino acid sequence of SEQ ID NO: 2 (Q31-z7);
(a3) an antibody H chain consisting of the amino acid sequence of SEQ ID NO: 3 (Q64-z55);
(a4) an antibody H chain consisting of the amino acid sequence of SEQ ID NO: 10 (Q64-z7);
(a5) an antibody H chain consisting of the amino acid sequence of SEQ ID NO: 11 (Q85-G4k);
(a6) an antibody H chain consisting of the amino acid sequence of SEQ ID NO: 12 (Q153-G4k);
(a7) an antibody H chain consisting of the amino acid sequence of SEQ ID NO: 13 (Q354-z106);
(a8) an antibody H chain consisting of the amino acid sequence of SEQ ID NO: 14 (Q360-G4k);
(a9) an antibody H chain consisting of the amino acid sequence of SEQ ID NO: 15 (Q360-z118);
(a10) an antibody H chain consisting of the amino acid sequence of SEQ ID NO: 16 (Q405-G4k);
(a11) an antibody H chain consisting of the amino acid sequence of SEQ ID NO: 17 (Q458-z106);
(a12) an antibody H chain consisting of the amino acid sequence of SEQ ID NO: 18 (Q460-z121);
(a13) an antibody H chain consisting of the amino acid sequence of SEQ ID NO: 19 (Q499-z118);
(a14) an antibody H chain consisting of the amino acid sequence of SEQ ID NO: 20 (Q499-z121);
(b1) an antibody H chain consisting of the amino acid sequence of SEQ ID NO: 4 (J268-G4h);
(b2) an antibody H chain consisting of the amino acid sequence of SEQ ID NO: 5 (J321-G4h);
(b3) an antibody H chain consisting of the amino acid sequence of SEQ ID NO: 6 (J326-z107);
(b4) an antibody H chain consisting of the amino acid sequence of SEQ ID NO: 7 (J344-z107);
(b5) an antibody H chain consisting of the amino acid sequence of SEQ ID NO: 21 (J232-G4h);
(b6) an antibody H chain consisting of the amino acid sequence of SEQ ID NO: 22 (J259-z107);
(b7) an antibody H chain consisting of the amino acid sequence of SEQ ID NO: 23 (J300-z107);
(b8) an antibody H chain consisting of the amino acid sequence of SEQ ID NO: 24 (J327-z107);
(b9) an antibody H chain consisting of the amino acid sequence of SEQ ID NO: 25 (J327-z119);
(b10) an antibody H chain consisting of the amino acid sequence of SEQ ID NO: 26 (J339-z119);
(b11) an antibody H chain consisting of the amino acid sequence of SEQ ID NO: 27 (J346-z107);
(b12) an antibody H chain consisting of the amino acid sequence of SEQ ID NO: 170 (J142-G4h);
(c1) an antibody L chain consisting of the amino acid sequence of SEQ ID NO: 8 (L2-k);
(c2) an antibody L chain consisting of the amino acid sequence of SEQ ID NO: 9 (L45-k);
(c3) an antibody L chain consisting of the amino acid sequence of SEQ ID NO: 28 (L248-k);
(c4) an antibody L chain consisting of the amino acid sequence of SEQ ID NO: 29 (L324-k);
(c5) an antibody L chain consisting of the amino acid sequence of SEQ ID NO: 30 (L334-k);
(c6) an antibody L chain consisting of the amino acid sequence of SEQ ID NO: 31 (L377-k);
(c7) an antibody L chain consisting of the amino acid sequence of SEQ ID NO: 32 (L404-k);
(c8) an antibody L chain consisting of the amino acid sequence of SEQ ID NO: 33 (L406-k);
(c9) an antibody L chain consisting of the amino acid sequence of SEQ ID NO: 34 (L408-k); and
(c10) an antibody L chain consisting of the amino acid sequence of SEQ ID NO: 171 (L180-k).

11. The multispecific antigen-binding molecule of claim 1, wherein the multispecific antigen-binding molecule is a multispecific antibody.

12. A bispecific antibody of any one of the following (a) to (u):
(a) a bispecific antibody (Q1-G4k/J268-G4h/L45-k), wherein the first polypeptide is an H chain consisting of the amino acid sequence of SEQ ID NO: 1, the second polypeptide is an H chain consisting of the amino acid sequence of SEQ ID NO: 4, and the third polypeptide and the fourth polypeptide are a commonly shared L chain of SEQ ID NO: 9;
(b) a bispecific antibody (Q1-G4k/J321-G4h/L45-k), wherein the first polypeptide is an H chain consisting of the amino acid sequence of SEQ ID NO: 1, the second polypeptide is an H chain consisting of the amino acid sequence of SEQ ID NO: 5, and the third polypeptide and the fourth polypeptide are a commonly shared L chain of SEQ ID NO: 9;
(c) a bispecific antibody (Q31-z7/J326-z107/L2-k), wherein the first polypeptide is an H chain consisting of the amino acid sequence of SEQ ID NO: 2, the second polypeptide is an H chain consisting of the amino acid sequence of SEQ ID NO: 6, and the third polypeptide and the fourth polypeptide are a commonly shared L chain of SEQ ID NO: 8;
(d) a bispecific antibody (Q64-z55/J344-z107/L45-k), wherein the first polypeptide is an H chain consisting of the amino acid sequence of SEQ ID NO: 3, the second polypeptide is an H chain consisting of the amino acid sequence of SEQ ID NO: 7, and the third polypeptide and the fourth polypeptide are a commonly shared L chain of SEQ ID NO: 9;
(e) a bispecific antibody (Q64-z7/J326-z107/L334-k), wherein the first polypeptide is an H chain consisting of the amino acid sequence of SEQ ID NO: 10, the second polypeptide is an H chain consisting of the amino acid sequence of SEQ ID NO: 6, and the third polypeptide and the fourth polypeptide are a commonly shared L chain of SEQ ID NO: 30;
(f) a bispecific antibody (Q64-z7/J344-z107/L406-k), wherein the first polypeptide is an H chain consisting of the amino acid sequence of SEQ ID NO: 10, the second polypeptide is an H chain consisting of the amino acid sequence of SEQ ID NO: 7, and the third polypeptide and the fourth polypeptide are a commonly shared L chain of SEQ ID NO: 33;
(g) a bispecific antibody (Q85-G4k/J268-G4h/L406-k), wherein the first polypeptide is an H chain consisting of the amino acid sequence of SEQ ID NO: 11, the second polypeptide is an H chain consisting of the amino acid sequence of SEQ ID NO: 4, and the third polypeptide and the fourth polypeptide are a commonly shared L chain of SEQ ID NO: 33;
(h) a bispecific antibody (Q85-G4k/J321-G4h/L334-k), wherein the first polypeptide is an H chain consisting of the amino acid sequence of SEQ ID NO: 11, the second polypeptide is an H chain consisting of the amino acid sequence of SEQ ID NO: 5, and the third polypeptide and the fourth polypeptide are a commonly shared L chain of SEQ ID NO: 30;
(i) a bispecific antibody (Q153-G4k/J232-G4h/L406-k), wherein the first polypeptide is an H chain consisting of the amino acid sequence of SEQ ID NO: 12, the second polypeptide is an H chain consisting of the amino acid sequence of SEQ ID NO: 21, and the third polypeptide and the fourth polypeptide are a commonly shared L chain of SEQ ID NO: 33;
(j) a bispecific antibody (Q354-z106/J259-z107/L324-k), wherein the first polypeptide is an H chain consisting of the amino acid sequence of SEQ ID NO: 13, the second polypeptide is an H chain consisting of the amino acid sequence of SEQ ID NO: 22, and the third polypeptide and the fourth polypeptide are a commonly shared L chain of SEQ ID NO: 29;
(k) a bispecific antibody (Q360-G4k/J232-G4h/L406-k), wherein the first polypeptide is an H chain consisting of the amino acid sequence of SEQ ID NO: 14, the second polypeptide is an H chain consisting of the amino acid sequence of SEQ ID NO: 21, and the third polypeptide and the fourth polypeptide are a commonly shared L chain of SEQ ID NO: 33;
(l) a bispecific antibody (Q360-z118/J300-z107/L334-k), wherein the first polypeptide is an H chain consisting of the amino acid sequence of SEQ ID NO: 15, the second polypeptide is an H chain consisting of the amino acid sequence of SEQ ID NO: 23, and the third polypeptide and the fourth polypeptide are a commonly shared L chain of SEQ ID NO: 30;
(m) a bispecific antibody (Q405-G4k/J232-G4h/L248-k), wherein the first polypeptide is an H chain consisting of the amino acid sequence of SEQ ID NO: 16, the second polypeptide is an H chain consisting of the amino acid sequence of SEQ ID NO: 21, and the third polypeptide and the fourth polypeptide are a commonly shared L chain of SEQ ID NO: 28;
(n) a bispecific antibody (Q458-z106/J346-z107/L408-k), wherein the first polypeptide is an H chain consisting of the amino acid sequence of SEQ ID NO: 17, the second polypeptide is an H chain consisting of the amino acid sequence of SEQ ID NO: 27, and the third polypeptide and the fourth polypeptide are a commonly shared L chain of SEQ ID NO: 34;
(o) a bispecific antibody (Q460-z121/J327-z119/L334-k), wherein the first polypeptide is an H chain consisting of the amino acid sequence of SEQ ID NO: 18, the second polypeptide is an H chain consisting of the amino acid sequence of SEQ ID NO: 25, and the third polypeptide and the fourth polypeptide are a commonly shared L chain of SEQ ID NO: 30;
(p) a bispecific antibody (Q499-z118/J327-z107/L334-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 30;
(q) a bispecific antibody (Q499-z118/J327-z107/L377-k), wherein the first polypeptide is an H chain consisting of the amino acid sequence of SEQ ID NO: 19, the second polypeptide is an H chain consisting of the amino acid sequence of SEQ ID NO: 24, and the third polypeptide and the fourth polypeptide are a commonly shared L chain of SEQ ID NO: 31;
(r) a bispecific antibody (Q499-z118/J346-z107/L248-k), wherein the first polypeptide is an H chain consisting of the amino acid sequence of SEQ ID NO: 19, the second polypeptide is an H chain consisting of the amino acid sequence of SEQ ID NO: 27, and the third polypeptide and the fourth polypeptide are a commonly shared L chain of SEQ ID NO: 28;
(s) a bispecific antibody (Q499-z121/J327-z119/L404-k), wherein the first polypeptide is an H chain consisting of the amino acid sequence of SEQ ID NO: 20, the second polypeptide is an H chain consisting of the amino acid sequence of SEQ ID NO: 25, and the third polypeptide and the fourth polypeptide are a commonly shared L chain of SEQ ID NO: 32;
(t) a bispecific antibody (Q499-z121/J339-z119/L377-k), wherein the first polypeptide is an H chain consisting of the amino acid sequence of SEQ ID NO: 20, the second polypeptide is an H chain consisting of the amino acid sequence of SEQ ID NO: 26, and the third polypeptide and the fourth polypeptide are a commonly shared L chain of SEQ ID NO: 31; and
(u) a bispecific antibody (Q153-G4k/J142-G4h/L180-k), wherein the first polypeptide is an H chain consisting of the amino acid sequence of SEQ ID NO: 12, the second polypeptide is an H chain consisting of the amino acid sequence of SEQ ID NO: 170, and the third polypeptide and the fourth polypeptide are a commonly shared L chain of SEQ ID NO: 171.

13. A nucleic acid encoding the multispecific antigen-binding molecule of claim 1.

14. A vector inserted with the nucleic acid of claim 13.

15. A cell comprising the nucleic acid of claim 13.

16. A method for producing a multispecific antigen-binding molecule by culturing the cell of claim 15.


SEQUENCE LISTING (179 sequences)

SEQ ID NO: 1 - protein, 448 aa, synthetic construct
  REGION 1..448: note = artificial sequence
QVQLVESGGGLVQPGGSLRLSCAASGFTFSYYDMAWVRQAPGKGLEWVASISPSGGSTYYRHSVKGRFTVSRDNAKNSLYLQMNSLRAEDTAVYYCARRAGHNLGAGWYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 2 - protein, 448 aa, synthetic construct
  REGION 1..448: note = artificial sequence
QVQLVESGGGLVQPGGSLRLSCAASGFTFSYYDMAWVRQAPGKGLEWVASISPSGGSTYYRRSVKGRFTVSRDNAKNSLYLQMNSLRAEDTAVYYCARRAGHNLGAGWYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 3 - protein, 448 aa, synthetic construct
  REGION 1..448: note = artificial sequence
QVQLVESGGGLVQPGGSLRLSCAASGFTFSYYDMAWVRQAPGKGLEWVASISPSGGSTYYRRSVKGRFTVSRDNAKNSLYLQMNSLRAEDTAVYYCARRAGHNFGAGWYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 4 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTGTAYMELSSLRSEDTAVYHCARRKSRGYHLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 5 - protein, 444 aa, synthetic construct
  REGION 1..444: note = artificial sequence
QVQLVQSGSELKKPGASVKVSCKASGYTFTDNNMHWVRQAPGQGLEWMGDINTRSGGSIYNEEFQDRVIMTVDKSTDTAYMELSSLRSEDTAVYHCARRKSYGYHLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 6 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTAVYHCARRKSYGNHLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRYTQESLSLSP

SEQ ID NO: 7 - protein, 444 aa, synthetic construct
  REGION 1..444: note = artificial sequence
QVQLVQSGSELKKPGASVKVSCKASGYTFTDNNMDWVRQAPGQGLEWMGDINTKSGGSIYNEEFQDRVIMTVDKSTDTAYMELSSLRSEDTATYHCARRQSYGYHLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRYTQESLSLSP

SEQ ID NO: 8 - protein, 213 aa, synthetic construct
  REGION 1..213: note = artificial sequence
DIQMTQSPSSLSASVGDRVTITCKASQNIYKNLAWYQQKPGQAPKLLIYSASYRYSGVPDRFSGSRYGTDFTLTISSLQPEDLATYYCQQYYSGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, synthetic construct
  REGION 1..214: note = artificial sequence
DIQMTQSPSSLSASVGDRVTITCKASQNIYKNLAWYQQKPGQAPKLLIYSASYRYSGVPDRFSGSRYGTDFTLTISSLQPEDLATYYCQQYYSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 448 aa, synthetic construct
  REGION 1..448: note = artificial sequence
QVQLVESGGGLVQPGGSLRLSCAASGFTFSYYDMAWVRQAPGKGLEWVASISPSGGSTYYRRSVKGRFTVSRDNAKNSLYLQMNSLRAEDTAVYYCARRAGHNFGAGWYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 11 - protein, 448 aa, synthetic construct
  REGION 1..448: note = artificial sequence
QVQLVESGGGLVQPGGSLRLSCAASGFTFSYYDMAWVRQAPGKGLEWVASISPSGGSTYYRRSVKGRFTVSRDNAKNSLYLQMNSLRAEDTAVYYCARRAGHNYGAGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 12 - protein, 448 aa, synthetic construct
  REGION 1..448: note = artificial sequence
QVQLVESGGGLVQPGGSLRLSCAASGFTFSYYDINWVRQAPGKGLEWVASISPSGGSTYYRRSVKGRFTISRDNAKNSLYLQMNSLRAEDTAVYYCATRAGHNYGAGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 13 - protein, 448 aa, synthetic construct
  REGION 1..448: note = artificial sequence
QVQLVESGGGLVQPGGSLRLSCAASGFTFSYYDINWVRQAPGKGLEWVSSISPSGQSTYYRREVKGRFTISRDNSKNTLYLQMNSLRAEDTAVYYCARRSGHNYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSL

SEQ ID NO: 14 - protein, 448 aa, synthetic construct
  REGION 1..448: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRSGHNYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 15 - protein, 448 aa, synthetic construct
  REGION 1..448: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRSGHNYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 16 - protein, 448 aa, synthetic construct
  REGION 1..448: note = artificial sequence
QVQLVESGGGLVQPGGSLRLSCAASGFTFSYYDISWVRQAPGKGLEWVSSISPSGQSTYYRREVKGRFTISRDNSKNTLYLQMNSLRAEDTAVYYCARRSGHNFGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 17 - protein, 448 aa, synthetic construct
  REGION 1..448: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRSGKSYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSL

SEQ ID NO: 18 - protein, 448 aa, synthetic construct
  REGION 1..448: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRSGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 19 - protein, 448 aa, synthetic construct
  REGION 1..448: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 20 - protein, 448 aa, synthetic construct
  REGION 1..448: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 21 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTGTAYMELSSLRSEDTAVYYCARRKSYGYHLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 22 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTGTAYMELSSLRSEDTAVYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRYTQESLSLSP

SEQ ID NO: 23 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTAVYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRYTQESLSLSP

SEQ ID NO: 24 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRYTQESLSLSP

SEQ ID NO: 25 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 26 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDTSTDTAYMELSSLRSEDTATYHCARRKSYGYHLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 27 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYHLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRYTQESLSLSP

SEQ ID NO: 28 - protein, 214 aa, synthetic construct
  REGION 1..214: note = artificial sequence
DIQMTQSPSSLSASVGDRVTITCKASKNIERNLAWYQQKPGQAPRLLIYRASRKESGVPDRFSGSRYGTDFTLTISSLQPEDIATYYCQQYSSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, synthetic construct
  REGION 1..214: note = artificial sequence
DIQMTQSPSSLSASVGDRVTITCKASRNIERNLAWYQQKPGQAPELLIYRADRKESGVPDRFSGSRYGTDFTLTISSLQPEDIATYYCQQYSSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, synthetic construct
  REGION 1..214: note = artificial sequence
DIQMTQSPSSLSASVGDRVTITCKASRNIERNLAWYQQKPGQAPELLIYQASRKESGVPDRFSGSRYGTDFTLTISSLQPEDIATYYCQQYSSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, synthetic construct
  REGION 1..214: note = artificial sequence
DIQMTQSPSSLSASVGDRVTITCKASRNIERQLAWYQQKPGQAPELLIYQASRKESGVPDRFSGSRYGTDFTLTISSLQPEDIATYYCQQYSSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, synthetic construct
  REGION 1..214: note = artificial sequence
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 214 aa, synthetic construct
  REGION 1..214: note = artificial sequence
DIQMTQSPSSLSASVGDRVTITCKASRNIERQLAWYQQKPGQAPELLIYRASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, synthetic construct
  REGION 1..214: note = artificial sequence
DIQMTQSPSSLSASVGDRVTITCKASRNIERQLAWYQQKPGQAPELLIYRADRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 123 aa, synthetic construct
  REGION 1..123: note = artificial sequence
QVQLVESGGGLVQPGGSLRLSCAASGFTFSYYDMAWVRQAPGKGLEWVASISPSGGSTYYRHSVKGRFTVSRDNAKNSLYLQMNSLRAEDTAVYYCARRAGHNLGAGWYFDFWGQGTLVTVSS

SEQ ID NO: 36 - protein, 123 aa, synthetic construct
  REGION 1..123: note = artificial sequence
QVQLVESGGGLVQPGGSLRLSCAASGFTFSYYDMAWVRQAPGKGLEWVASISPSGGSTYYRRSVKGRFTVSRDNAKNSLYLQMNSLRAEDTAVYYCARRAGHNLGAGWYFDFWGQGTLVTVSS

SEQ ID NO: 37 - protein, 123 aa, synthetic construct
  REGION 1..123: note = artificial sequence
QVQLVESGGGLVQPGGSLRLSCAASGFTFSYYDMAWVRQAPGKGLEWVASISPSGGSTYYRRSVKGRFTVSRDNAKNSLYLQMNSLRAEDTAVYYCARRAGHNFGAGWYFDFWGQGTLVTVSS

SEQ ID NO: 38 - protein, 123 aa, synthetic construct
  REGION 1..123: note = artificial sequence
QVQLVESGGGLVQPGGSLRLSCAASGFTFSYYDMAWVRQAPGKGLEWVASISPSGGSTYYRRSVKGRFTVSRDNAKNSLYLQMNSLRAEDTAVYYCARRAGHNYGAGWYFDYWGQGTLVTVSS

SEQ ID NO: 39 - protein, 123 aa, synthetic construct
  REGION 1..123: note = artificial sequence
QVQLVESGGGLVQPGGSLRLSCAASGFTFSYYDINWVRQAPGKGLEWVASISPSGGSTYYRRSVKGRFTISRDNAKNSLYLQMNSLRAEDTAVYYCATRAGHNYGAGWYFDYWGQGTLVTVSS

SEQ ID NO: 40 - protein, 123 aa, synthetic construct
  REGION 1..123: note = artificial sequence
QVQLVESGGGLVQPGGSLRLSCAASGFTFSYYDINWVRQAPGKGLEWVSSISPSGQSTYYRREVKGRFTISRDNSKNTLYLQMNSLRAEDTAVYYCARRSGHNYGGGWYFDYWGQGTLVTVSS

SEQ ID NO: 41 - protein, 123 aa, synthetic construct
  REGION 1..123: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRSGHNYGGGWYFDYWGQGTLVTVSS

SEQ ID NO: 42 - protein, 123 aa, synthetic construct
  REGION 1..123: note = artificial sequence
QVQLVESGGGLVQPGGSLRLSCAASGFTFSYYDISWVRQAPGKGLEWVSSISPSGQSTYYRREVKGRFTISRDNSKNTLYLQMNSLRAEDTAVYYCARRSGHNFGGGWYFDYWGQGTLVTVSS

SEQ ID NO: 43 - protein, 123 aa, synthetic construct
  REGION 1..123: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRSGKSYGGGWYFDYWGQGTLVTVSS

SEQ ID NO: 44 - protein, 123 aa, synthetic construct
  REGION 1..123: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRSGREYGGGWYFDYWGQGTLVTVSS

SEQ ID NO: 45 - protein, 123 aa, synthetic construct
  REGION 1..123: note = artificial sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSS

SEQ ID NO: 46 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDKSTGTAYMELSSLRSEDTAVYYCARRKSYGYHLDEWGEGTLVTVSS

SEQ ID NO: 47 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDKSTGTAYMELSSLRSEDTAVYHCARRKSYGYYLDEWGEGTLVTVSS

SEQ ID NO: 48 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDKSTGTAYMELSSLRSEDTAVYHCARRKSRGYHLDEWGEGTLVTVSS

SEQ ID NO: 49 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTAVYHCARRKSYGYYLDEWGEGTLVTVSS

SEQ ID NO: 50 - protein, 119 aa, synthetic construct
  REGION 1..119: note = artificial sequence
QVQLVQSGSELKKPGASVKVSCKASGYTFTDNNMHWVRQAPGQGLEWMGDINTRSGGSIYNEEFQDRVIMTVDKSTDTAYMELSSLRSEDTAVYHCARRKSYGYHLDEWGEGTLVTVSS

SEQ ID NO: 51 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTAVYHCARRKSYGNHLDEWGEGTLVTVSS

SEQ ID NO: 52 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSS

SEQ ID NO: 53 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDTSTDTAYMELSSLRSEDTATYHCARRKSYGYHLDEWGEGTLVTVSS

SEQ ID NO: 54 - protein, 119 aa, synthetic construct
  REGION 1..119: note = artificial sequence
QVQLVQSGSELKKPGASVKVSCKASGYTFTDNNMDWVRQAPGQGLEWMGDINTKSGGSIYNEEFQDRVIMTVDKSTDTAYMELSSLRSEDTATYHCARRQSYGYHLDEWGEGTLVTVSS

SEQ ID NO: 55 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYHLDEWGEGTLVTVSS

SEQ ID NO: 56 - protein, 106 aa, synthetic construct
  REGION 1..106: note = artificial sequence
DIQMTQSPSSLSASVGDRVTITCKASQNIYKNLAWYQQKPGQAPKLLIYSASYRYSGVPDRFSGSRYGTDFTLTISSLQPEDLATYYCQQYYSGLTFGGGTKVEIK

SEQ ID NO: 57 - protein, 107 aa, synthetic construct
  REGION 1..107: note = artificial sequence
DIQMTQSPSSLSASVGDRVTITCKASQNIYKNLAWYQQKPGQAPKLLIYSASYRYSGVPDRFSGSRYGTDFTLTISSLQPEDLATYYCQQYYSPPLTFGGGTKVEIK

SEQ ID NO: 58 - protein, 107 aa, synthetic construct
  REGION 1..107: note = artificial sequence
DIQMTQSPSSLSASVGDRVTITCKASKNIERNLAWYQQKPGQAPRLLIYRASRKESGVPDRFSGSRYGTDFTLTISSLQPEDIATYYCQQYSSPPLTFGGGTKVEIK

SEQ ID NO: 59 - protein, 107 aa, synthetic construct
  REGION 1..107: note = artificial sequence
DIQMTQSPSSLSASVGDRVTITCKASRNIERNLAWYQQKPGQAPELLIYRADRKESGVPDRFSGSRYGTDFTLTISSLQPEDIATYYCQQYSSPPLTFGGGTKVEIK

SEQ ID NO: 60 - protein, 107 aa, synthetic construct
  REGION 1..107: note = artificial sequence
DIQMTQSPSSLSASVGDRVTITCKASRNIERNLAWYQQKPGQAPELLIYQASRKESGVPDRFSGSRYGTDFTLTISSLQPEDIATYYCQQYSSPPLTFGGGTKVEIK

SEQ ID NO: 61 - protein, 107 aa, synthetic construct
  REGION 1..107: note = artificial sequence
DIQMTQSPSSLSASVGDRVTITCKASRNIERQLAWYQQKPGQAPELLIYQASRKESGVPDRFSGSRYGTDFTLTISSLQPEDIATYYCQQYSSPPLTFGGGTKVEIK

SEQ ID NO: 62 - protein, 107 aa, synthetic construct
  REGION 1..107: note = artificial sequence
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIK

SEQ ID NO: 63 - protein, 107 aa, synthetic construct
  REGION 1..107: note = artificial sequence
DIQMTQSPSSLSASVGDRVTITCKASRNIERQLAWYQQKPGQAPELLIYRASRKESGVPDRFSGSRYGTDFTLTISSLQPEDIATYYCQQYSDPPLTFGGGTKVEIK

SEQ ID NO: 64 - protein, 107 aa, synthetic construct
  REGION 1..107: note = artificial sequence
DIQMTQSPSSLSASVGDRVTITCKASRNIERQLAWYQQKPGQAPELLIYRADRKESGVPDRFSGSRYGTDFTLTISSLQPEDIATYYCQQYSDPPLTFGGGTKVEIK

SEQ ID NO: 65 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 66 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 67 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 68 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSL

SEQ ID NO: 69 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 70 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 71 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 72 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNRYTQESLSLSP

SEQ ID NO: 73 - protein, 325 aa, synthetic construct
  REGION 1..325: note = artificial sequence
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 74 - protein, 107 aa, synthetic construct [sequence omitted]

SEQ ID NO: 75 - protein, 5 aa, synthetic construct
  REGION 1..5: note = artificial sequence
YYDMA

SEQ ID NO: 76 - protein, 17 aa, synthetic construct
  REGION 1..17: note = artificial sequence
SISPSGGSTYYRHSVKG

SEQ ID NO: 77 - protein, 14 aa, synthetic construct
  REGION 1..14: note = artificial sequence
RAGHNLGAGWYFDF

SEQ ID NO: 78 - protein, 5 aa, synthetic construct
  REGION 1..5: note = artificial sequence
YYDMA

SEQ ID NO: 79 - protein, 17 aa, synthetic construct
  REGION 1..17: note = artificial sequence
SISPSGGSTYYRRSVKG

SEQ ID NO: 80 - protein, 14 aa, synthetic construct
  REGION 1..14: note = artificial sequence
RAGHNLGAGWYFDF

SEQ ID NO: 81 - protein, 5 aa, synthetic construct
  REGION 1..5: note = artificial sequence
YYDMA

SEQ ID NO: 82 - protein, 17 aa, synthetic construct
  REGION 1..17: note = artificial sequence
SISPSGGSTYYRRSVKG

SEQ ID NO: 83 - protein, 14 aa, synthetic construct
  REGION 1..14: note = artificial sequence
RAGHNFGAGWYFDF

SEQ ID NO: 84 - protein, 5 aa, synthetic construct
  REGION 1..5: note = artificial sequence
YYDMA

SEQ ID NO: 85 - protein, 17 aa, synthetic construct
  REGION 1..17: note = artificial sequence
SISPSGGSTYYRRSVKG

SEQ ID NO: 86 - protein, 14 aa, synthetic construct
  REGION 1..14: note = artificial sequence
RAGHNYGAGWYFDY

SEQ ID NO: 87 - protein, 5 aa, synthetic construct
  REGION 1..5: note = artificial sequence
YYDIN

SEQ ID NO: 88 - protein, 17 aa, synthetic construct
  REGION 1..17: note = artificial sequence
SISPSGGSTYYRRSVKG

SEQ ID NO: 89 - protein, 14 aa, synthetic construct
  REGION 1..14: note = artificial sequence
RAGHNYGAGWYFDY

SEQ ID NO: 90 - protein, 5 aa, synthetic construct
  REGION 1..5: note = artificial sequence
YYDIN

SEQ ID NO: 91 - protein, 17 aa, synthetic construct
  REGION 1..17: note = artificial sequence
SISPSGQSTYYRREVKG

SEQ ID NO: 92 - protein, 14 aa, synthetic construct
  REGION 1..14: note = artificial sequence
RSGHNYGGGWYFDY

SEQ ID NO: 93 - protein, 5 aa, synthetic construct
  REGION 1..5: note = artificial sequence
YYDIQ

SEQ ID NO: 94 - protein, 17 aa, synthetic construct
  REGION 1..17: note = artificial sequence
SISPSGQSTYYRREVKG

SEQ ID NO: 95 - protein, 14 aa, synthetic construct
  REGION 1..14: note = artificial sequence
RSGHNYGGGWYFDY

SEQ ID NO: 96 - protein, 5 aa, synthetic construct
  REGION 1..5: note = artificial sequence
YYDIS

SEQ ID NO: 97 - protein, 17 aa, synthetic construct
  REGION 1..17: note = artificial sequence
SISPSGQSTYYRREVKG

SEQ ID NO: 98 - protein, 14 aa, synthetic construct
  REGION 1..14: note = artificial sequence
RSGHNFGGGWYFDY

SEQ ID NO: 99 - protein, 5 aa, synthetic construct
  REGION 1..5: note = artificial sequence
YYDIQ

SEQ ID NO: 100 - protein, 17 aa, synthetic construct
  REGION 1..17: note = artificial sequence
SISPSGQSTYYRREVKG

SEQ ID NO: 101 - protein, 14 aa, synthetic construct
  REGION 1..14: note = artificial sequence
RSGKSYGGGWYFDY

SEQ ID NO: 102 - protein, 5 aa, synthetic construct
  REGION 1..5: note = artificial sequence
YYDIQ

SEQ ID NO: 103 - protein, 17 aa, synthetic construct
  REGION 1..17: note = artificial sequence
SISPSGQSTYYRREVKG

SEQ ID NO: 104 - protein, 14 aa, synthetic construct
  REGION 1..14: note = artificial sequence
RSGREYGGGWYFDY

SEQ ID NO: 105 - protein, 5 aa, synthetic construct
  REGION 1..5: note = artificial sequence
YYDIQ

SEQ ID NO: 106 - protein, 17 aa, synthetic construct
  REGION 1..17: note = artificial sequence
SISPSGQSTYYRREVKG

SEQ ID NO: 107 - protein, 14 aa, synthetic construct
  REGION 1..14: note = artificial sequence
RTGREYGGGWYFDY

SEQ ID NO: 108 - protein, 5 aa, synthetic construct
  REGION 1..5: note = artificial sequence
DNNMD

SEQ ID NO: 109 - protein, 17 aa, synthetic construct
  REGION 1..17: note = artificial sequence
DINTRSGGSIYNEEFQD

SEQ ID NO: 110 - protein, 10 aa, synthetic construct
  REGION 1..10: note = artificial sequence
RKSYGYHLDE

SEQ ID NO: 111 - protein, 5 aa, synthetic construct
  REGION 1..5: note = artificial sequence
DNNMD

SEQ ID NO: 112 - protein, 17 aa, synthetic construct
  REGION 1..17: note = artificial sequence
DINTRSGGSIYNEEFQD

SEQ ID NO: 113 - protein, 10 aa, synthetic construct
  REGION 1..10: note = artificial sequence
RKSYGYYLDE

SEQ ID NO: 114 - protein, 5 aa, synthetic construct
  REGION 1..5: note = artificial sequence
DNNMD

SEQ ID NO: 115 - protein, 17 aa, synthetic construct
  REGION 1..17: note = artificial sequence
DINTRSGGSIYNEEFQD

SEQ ID NO: 116 - protein, 10 aa, synthetic construct
  REGION 1..10: note = artificial sequence
RKSRGYHLDE

SEQ ID NO: 117 - protein, 5 aa, synthetic construct
  REGION 1..5: note = artificial sequence
DNNMD

SEQ ID NO: 118 - protein, 17 aa, synthetic construct
  REGION 1..17: note = artificial sequence
DINTRSGGSIYNEEFQD

SEQ ID NO: 119 - protein, 10 aa, synthetic construct
  REGION 1..10: note = artificial sequence
RKSYGYYLDE

SEQ ID NO: 120 - protein, 5 aa, synthetic construct
  REGION 1..5: note = artificial sequence
DNNMH

SEQ ID NO: 121 - protein, 17 aa, synthetic construct
  REGION 1..17: note = artificial sequence
DINTRSGGSIYNEEFQD

SEQ ID NO: 122 - protein, 10 aa, synthetic construct
  REGION 1..10: note = artificial sequence
RKSYGYHLDE

SEQ ID NO: 123 - protein, 5 aa, synthetic construct
  REGION 1..5: note = artificial sequence
DNNMD

SEQ ID NO: 124 - protein, 17 aa, synthetic construct
  REGION 1..17: note = artificial sequence
DINTRSGGSIYNEEFQD

SEQ ID NO: 125 - protein, 10 aa, synthetic construct
  REGION 1..10: note = artificial sequence
RKSYGNHLDE

SEQ ID NO: 126 - protein, 5 aa, synthetic construct
  REGION 1..5: note = artificial sequence
DNNMD

SEQ ID NO: 127 - protein, 17 aa, synthetic construct
  REGION 1..17: note = artificial sequence
DINTRSGGSIYNEEFQD

SEQ ID NO: 128 - protein, 10 aa, synthetic construct
  REGION 1..10: note = artificial sequence
RKSYGYYLDE

SEQ ID NO: 129 - protein, 5 aa, synthetic construct
  REGION 1..5: note = artificial sequence
DNNMD

SEQ ID NO: 130 - protein, 17 aa, synthetic construct
  REGION 1..17: note = artificial sequence
DINTRSGGSIYNEEFQD

SEQ ID NO: 131 - protein, 10 aa, synthetic construct
  REGION 1..10: note = artificial sequence
RKSYGYHLDE

SEQ ID NO: 132 - protein, 5 aa, synthetic construct
  REGION 1..5: note = artificial sequence
DNNMD

SEQ ID NO: 133 - protein, 17 aa, synthetic construct
  REGION 1..17: note = artificial sequence
DINTKSGGSIYNEEFQD

SEQ ID NO: 134 - protein, 10 aa, synthetic construct
  REGION 1..10: note = artificial sequence
RQSYGYHLDE

SEQ ID NO: 135 - protein, 5 aa, synthetic construct
  REGION 1..5: note = artificial sequence
DNNMD

SEQ ID NO: 136 - protein, 17 aa, synthetic construct
  REGION 1..17: note = artificial sequence
DINTRSGGSIYNEEFQD

SEQ ID NO: 137 - protein, 10 aa, synthetic construct
  REGION 1..10: note = artificial sequence
RKSYGYHLDE

SEQ ID NO: 138 - protein, 11 aa, synthetic construct
  REGION 1..11: note = artificial sequence
KASQNIYKNLA

SEQ ID NO: 139 - protein, 7 aa, synthetic construct
  REGION 1..7: note = artificial sequence
SASYRYS

SEQ ID NO: 140 - protein, 8 aa, synthetic construct
  REGION 1..8: note = artificial sequence
QQYYSGLT

SEQ ID NO: 141 - protein, 11 aa, synthetic construct
  REGION 1..11: note = artificial sequence
KASQNIYKNLA

SEQ ID NO: 142 - protein, 7 aa, synthetic construct
  REGION 1..7: note = artificial sequence
SASYRYS

SEQ ID NO: 143 - protein, 9 aa, synthetic construct
  REGION 1..9: note = artificial sequence
QQYYSPPLT

SEQ ID NO: 144 - protein, 11 aa, synthetic construct
  REGION 1..11: note = artificial sequence
KASKNIERNLA

SEQ ID NO: 145 - protein, 7 aa, synthetic construct
  REGION 1..7: note = artificial sequence
RASRKES

SEQ ID NO: 146 - protein, 9 aa, synthetic construct
  REGION 1..9: note = artificial sequence
QQYSSPPLT

SEQ ID NO: 147 - protein, 11 aa, synthetic construct
  REGION 1..11: note = artificial sequence
KASRNIERNLA

SEQ ID NO: 148 - protein, 7 aa, synthetic construct
  REGION 1..7: note = artificial sequence
RADRKES

SEQ ID NO: 149 - protein, 9 aa, synthetic construct
  REGION 1..9: note = artificial sequence
QQYSSPPLT

SEQ ID NO: 150 - protein, 11 aa, synthetic construct
  REGION 1..11: note = artificial sequence
KASRNIERNLA

SEQ ID NO: 151 - protein, 7 aa, synthetic construct
  REGION 1..7: note = artificial sequence
QASRKES

SEQ ID NO: 152 - protein, 9 aa, synthetic construct
  REGION 1..9: note = artificial sequence
QQYSSPPLT

SEQ ID NO: 153 - protein, 11 aa, synthetic construct
  REGION 1..11: note = artificial sequence
KASRNIERQLA

SEQ ID NO: 154 - protein, 7 aa, synthetic construct
  REGION 1..7: note = artificial sequence
QASRKES

SEQ ID NO: 155 - protein, 9 aa, synthetic construct
  REGION 1..9: note = artificial sequence
QQYSSPPLT

SEQ ID NO: 156 - protein, 11 aa, synthetic construct
  REGION 1..11: note = artificial sequence
KASRNIERQLA

SEQ ID NO: 157 - protein, 7 aa, synthetic construct
  REGION 1..7: note = artificial sequence
QASRKES

SEQ ID NO: 158 - protein, 9 aa, synthetic construct
  REGION 1..9: note = artificial sequence
QQYSDPPLT

SEQ ID NO: 159 - protein, 11 aa, synthetic construct
  REGION 1..11: note = artificial sequence
KASRNIERQLA

SEQ ID NO: 160 - protein, 7 aa, synthetic construct
  REGION 1..7: note = artificial sequence
RASRKES

SEQ ID NO: 161 - protein, 9 aa, synthetic construct
  REGION 1..9: note = artificial sequence
QQYSDPPLT

SEQ ID NO: 162 - protein, 11 aa, synthetic construct
  REGION 1..11: note = artificial sequence
KASRNIERQLA

SEQ ID NO: 163 - protein, 7 aa, synthetic construct
  REGION 1..7: note = artificial sequence
RADRKES

SEQ ID NO: 164 - protein, 9 aa, synthetic construct
  REGION 1..9: note = artificial sequence
QQYSDPPLT

SEQ ID NO: 165 - protein, 118 aa, Homo sapiens
EVQLVQSGAEVQKPGASVKVSCKASGYTFTDYYMHWVRQAPGEGLEWMGYINPSSGYTKYNRKFRDRVTITADKSTSTAYMELSSLRSEDTAVYYCARGGQGYYLDYWGEGTTVTVSS

SEQ ID NO: 166 - protein, 119 aa, Homo sapiens
EVQLVQSGAQVKKPGASVKVSCKASGYTFSDNNMDWVRQAPGKGLEWMGDINTKSGGSIYNQKFKGRVIMTIDKSTGTAYMELRSLRSDDTAIYYCARRRSYGYYFDYWGRGTLVTVSS

SEQ ID NO: 167 - protein, 106 aa, Homo sapiens
DIVMTQSPSSLSASVGDRVTITCKASQNVGTAVAWYQQKPGKAPKLLIYSASYRASGVPSRFSGSRYGTDFTLTISSLQPEDLATYYCQQYSNYITFGQGTKVEIK

SEQ ID NO: 168 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 169 - protein, 107 aa, Homo sapiens [sequence omitted]

SEQ ID NO: 170 - protein, 444 aa, synthetic construct
  REGION 1..444: note = artificial sequence
QVQLVQSGSELKKPGASVKVSCKASGYTFTDNNMDWVRQAPGQGLEWMGDINTRSGGSIYNEEFQDRVTMTIDKSTGTAYMELSSLRSEDTAVYYCARRRSYGYYHDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 171 - protein, 214 aa, synthetic construct
  REGION 1..214: note = artificial sequence
DIQMTQSPSSLSASVGDRVTITCKASRNIERNLAWYQQKPGQAPELLIYSASRKESGVPDRFSGSRYGTDFTLTISSLQPEDLATYYCQQYYSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 119 aa, synthetic construct
  REGION 1..119: note = artificial sequence
QVQLVQSGSELKKPGASVKVSCKASGYTFTDNNMDWVRQAPGQGLEWMGDINTRSGGSIYNEEFQDRVTMTIDKSTGTAYMELSSLRSEDTAVYYCARRRSYGYYHDEWGEGTLVTVSS

SEQ ID NO: 173 - protein, 107 aa, synthetic construct
  REGION 1..107: note = artificial sequence
DIQMTQSPSSLSASVGDRVTITCKASRNIERNLAWYQQKPGQAPELLIYSASRKESGVPDRFSGSRYGTDFTLTISSLQPEDLATYYCQQYYSPPLTFGGGTKVEIK

SEQ ID NO: 174 - protein, 5 aa, synthetic construct
  REGION 1..5: note = artificial sequence
DNNMD

SEQ ID NO: 175 - protein, 17 aa, synthetic construct
  REGION 1..17: note = artificial sequence
DINTRSGGSIYNEEFQD

SEQ ID NO: 176 - protein, 10 aa, synthetic construct
  REGION 1..10: note = artificial sequence
RRSYGYYHDE

SEQ ID NO: 177 - protein, 11 aa, synthetic construct
  REGION 1..11: note = artificial sequence
KASRNIERNLA

SEQ ID NO: 178 - protein, 7 aa, synthetic construct
  REGION 1..7: note = artificial sequence
SASRKES

SEQ ID NO: 179 - protein, 9 aa, synthetic construct
  REGION 1..9: note = artificial sequence
QQYYSPPLT